(19) United States
(12) Patent Application Publication
(10) Pub. No.: US 2007/0111227 A1
(43) Pub. Date: May 17, 2007

(54) Small regulatory RNAs and methods of use

(76) Inventors: Pamela J. Green, Newark, DE (US); Blake Meyers, Wilmington, DE (US); Cheng Lu, Newark, DE (US); Shivakundan Singh Tej, Herndon, VA (US); Frederic F. Souret, Newark, DE (US)

Correspondence Address: MCCARTER & ENGLISH, LLP;BASIL S. KRIKELIS, CITIZENS BANK CENTER, 919 N. MARKET STREET, SUITE 1800, WILMINGTON, DE, 19801, US

(21) Appl. No.: 11/495,951

(22) Filed: Jul. 28, 2006

Related U.S. Application Data
(60) Provisional application No. 60/703,215, filed on Jul. 28, 2005.
(60) Provisional application No. 60/772,666, filed on Feb. 13, 2006.

(51) Int. Cl. C12Q 1/68; G06F 19/00; C40B 40/08

(52) U.S. Cl. 435/6; 702/20

(57) ABSTRACT

The present invention relates to unique small ribonucleic acid molecules, for example siRNAs and miRNAs, identified and isolated using MPSS. Specifically, the invention is directed to the identification of a library of unique small RNA sequences from Arabidopsis thaliana. In another aspect, the small RNA sequences themselves are useful for performing biological functions, such as for example, RNA interference.

### CROSS-REFERENCE TO RELATED APPLICATIONS

Under 35 U.S.C. § 119(e) this application claims the benefit of U.S. Provisional Application No. 60/703,215, filed Jul. 28, 2005; and U.S. Provisional Application No. 60/772,666, filed Feb. 13, 2006, which are hereby incorporated by reference in their entirety and for all purposes.

### RELATED FEDERALLY SPONSORED RESEARCH

The work described in this application was sponsored by the NSF SGER under Contract Number 0439186; with additional support from the NSF Plant Genome Program Grant Number 0321437 (B.C.M) DOE DE-FG02-04ER15541 (P.J.G.) and NIH P20RR16472-04.

### SEQUENCE LISTING

The instant application contains a “lengthy” Sequence Listing of SEQ ID NOs: 1-185,413 which has been submitted via CD-Rs in lieu of a printed paper copy, and is hereby incorporated by reference in its entirety. Said CD-R, recorded on Jul. 28, 2006, is labeled “CRF”, “Copy 1” and “Copy 2”, respectively, and each contains only one identical 27.5 MB file (99689009. APP).

### FIELD OF THE INVENTION

The present invention relates generally to the isolation and identification of small ribonucleic acids (RNAs) from an organism and methods for their use. In particular the invention relates to novel small inhibitory RNAs (siRNAs), microRNAs (miRNAs), tiny RNAs or combinations thereof from an organism, for example, Arabidopsis thaliana. In a related aspect the invention relates to methods of using the small RNAs disclosed herein.

### BACKGROUND

Small ribonucleic acid (RNA) molecules are short RNA sequences (e.g., 15 to 30 nucleotides in size, but generally 21-24 nucleotides in size) that are produced by nearly all eukaryotes (e.g., fungi, plants, and animals). However, rather than encoding a protein, small RNAs function to reduce the mRNA abundance or protein abundance of the gene which is the “target.” In certain instances small RNAs can also result in target gene regulation by affecting chromatin structure. The two major types of small RNAs are known as small interfering RNAs (siRNAs) and microRNAs (miRNAs). Both types of molecules are processed from double-stranded RNA by RNase III enzymes called DICERs. Although relatively short in length, 15 to 30 nucleotides, small RNAs typically correspond to a single location in the host genome.

Small RNAs do not necessarily demonstrate perfect base pair complementarity with their target RNA. This phenomena allows for a single small RNA to interact with multiple targets such as those encoded by members of a gene family that share short regions of similarity. Therefore, although small RNAs may not match perfectly to their targets (i.e., they contain one or more base-pair mismatches) they retain the ability to direct cleavage or inhibit translation of the target mRNAs.

While similar in size, the biogenesis and function of siRNAs and miRNAs can be substantially different. For instance, siRNAs are processed from longer double-stranded RNA molecules and represent both strands of the RNA. In addition, siRNAs are incorporated into a multi-protein complex known as the RNA-induced silencing complex (RISC), where they can act as guides to target and degrade complementary mRNA molecules. In some systems, siRNAs can also trigger transcriptional silencing by guiding nuclear complexes that target either histone modifications or DNA methylation or both.

MicroRNA molecules, on the other hand, originate from distinct genomic loci predicted to encode transcripts that form ‘hairpin’ structures. These small RNAs, which are derived from one strand of the hairpin, guide the RISC (or a similar RNA-protein complex) to specific RNAs, such as mRNAs by forming base-pairing interactions. Like siRNA, miRNAs can induce cleavage and accelerate degradation of the mRNA targets. A second mechanism by which miRNAs affect gene function is to reduce or prevent mRNA translation and thereby limit protein production.

However, not all small RNAs fit precisely into these two categories. For example, trans-acting siRNAs (ta-siRNAs), recently found in plants, are technically siRNAs because they require the action of an RNA-dependent RNA polymerase to generate their double-stranded RNA precursors. After the ta-siRNAs are formed by cleavage of the double-stranded RNA by a DICER enzyme, they act like miRNAs to silence genes in trans that usually have little resemblance to the genes from which they derive (Vasquez et al, 2004; Peragine et al., 2004). Work in plants also led to a new model for the evolution of miRNA genes from inverted duplication of target genes. Founder genes formed by these initial inversions are thought to produce siRNAs that are replaced by miRNA as the sequence of the founder genes diverges (Allen et al., 2004).

As indicated above, small RNAs have many roles in organisms. For example, miRNAs are critical for development in both plants and animals. The first miRNAs were discovered for their role in the development of the nematode Caenorhabditis elegans (Lee at al., 1993). Numerous diverse examples have emerged subsequently including important roles of miRNAs in brain development in vertebrates and flower development in plants. Other studies have associated miRNA metabolism with cancer, and other human diseases. Small RNAs have also been associated with stress responses, hormonal responses, reproductive development, and small RNA metabolism. Endogenous siRNAs are also thought to function in part to protect the genome against damage or invasion by mobile genetic elements such as retro-transposons and viruses, which produce aberrant RNA or dsRNA in the host cell when they become active. It is well known however, that small RNA function can have profound effects on cellular physiology as well as the overall phenotype. Yet, these and other numerous examples likely represent only a subset of the roles of these molecules in eukaryotes. In theory they could regulate any gene so they could contribute to any biological function in an organism. Conversely, inhibiting elevating, or otherwise modulating the level of a given small RNA is a means of creating new advantageous traits. For example, modulating the expression of certain genes in a plant could affect its tolerance to pesticides, temperature, or soil conditions.

Currently, the typical method for the isolation and identification of small RNAs involves cloning, either as single molecules or “concatamers,” and subsequent sequencing by standard methods. Using this approach, a modest number of small RNA sequences have been identified from, for example, human, Drosophila melanogaster, mouse, Caenorhabditis elegans, and Arabidopsis thaliana. Obviously, these methods do not sequence deeply enough to sample the full complexity of small RNAs in plant and animal systems. While modern microarray-based methods for the quantification of small RNA abundance offer advantages of scale, they are relatively new, and their sensitivity and specificity have yet to be fully characterized. Therefore, most current analyses rely on RNA gel blots or assays with oligonucleotide probes that only detect individual or closely related small RNA sequences.

Recently, we demonstrated a method of performing massively parallel signature sequencing™ (“MPSS”) to sequence more than two million small RNAs from seedlings and the inflorescence stage of the model plant Arabidopsis thaliana. This method is the subject of U.S. patent application Ser. Nos. 11/204,903, which is incorporated herein by reference in its entirety. This technique allows for the efficient identification and isolation of many hundreds of thousands of individual sequences, the generation of a “library” of small RNAs. The abundance or frequency of occurrence of each distinct sequence from a small RNA “library” is indicative of the quantity in the original tissue from which the RNA was obtained. Moreover, by comparison of the signature sequences, which are typically 17-20 nucleotides in length, to a genomic DNA database it is possible to determine the locations on the DNA that serve as sources for the small RNAs. Comparisons to genome annotations, cDNA databases, and other data can often be used to identify the larger RNA precursors of the small RNAs. Most significantly, MPSS provides the ability to address small RNA biology on a genome-wide scale.

While, MPSS provides extraordinary depth, sequencing a half million or more molecules per library, utilizing another parallel sequencing approach, the 454 technology Margulies, M., et al., 2005. Genome sequencing in microfabricated high-density picolitre reactors. Nature 437: 376-380, provides longer reads and thereby provides information about length. Both methods provide quantitative data based on the frequency of the molecules that are sequenced. However, without identification, it is impossible to discover the functional significance of a given small RNA.

Interestingly, the small RNA population in plants may be among the most complex because, in addition to producing microRNAs (miRNAs) that play critical role in various developmental, stress, and signaling responses Chen, X., et al., 2005. MicroRNA Biogenesis and Function In Plants. FEBS Lett 579: 5923-5931; Zhang, B., et al., 2006, Conservation and Divergence of Plant MicroRNA Genes. Plant J 46: 243-259, plants also produce a complex set of small interfering RNAs (siRNAs); Vaucheret, H., et al., 2006, AGO1 Homeostasis Entails Coexpression of MIR168 and AGO1 and Preferential Stabilization of miR168 by AGO1. Mol Cell 22: 129-136. Among the approximately 77,000 different small RNAs that have been sequenced from Arabidopsis, it is likely that miRNAs account for less than 10%, so the non-redundant set of siRNAs must number more than 70,000 Lu, C., et al., 2005, Elucidation of the Small RNA Component of the Transcriptome. Science 309: 1567-1569. Most of these siRNAs match to repeated sequences such as transposons and retrotransposons. Thus, in cereals and other plant species with larger genomes and correspondingly higher contents of repeated DNA, the complexity of siRNAs is expected to be far greater.

While the ‘upstream’ biochemical steps that produce small RNAs have been relatively well characterized much remains to be understood about the complexity, abundance, targeting, and regulatory function of small RNAs. Because the search for these small RNAs has only occurred in the last 5 to 7 years, and because no methods prior to our invention permitted the large-scale characterization of these molecules (see U.S. Ser. No. 11/204,903), their ‘downstream’ role in many aspects of biology, and commercial utility has been poorly explored.

In addition to the transcriptional or post-transcriptional gene regulatory mechanisms that are mediated by small RNAs made within an organism (endogenous small RNAs), small RNAs can also be useful for purposes of RNA interference (RNAi). RNAi refers to the specific silencing of genes which bear substantial homology in nucleic acid sequence to small RNAs that are introduced or engineered to be produced within an organism, cell, or cell-free experimental system. RNAi is a process that appears to be conserved in eukaryotic cells across evolutionary lines, and involves some of the same cellular components and mechanisms involved in the small RNA mediated gene regulation mechanisms. For example, U.S. Pat. No. 7,022,828 to McSwiggen, which is incorporated herein by reference in its entirety, is one of the first patents to describe a small RNA molecule useful as an RNAi therapeutic for modulating immune responses in an animal.

In addition to therapeutic uses, there exists an overwhelming need for agents having agricultural applications, for example, to modify disease and pesticide resistance, and/or enhance plant growth, nutritional value, abundance, etc . . . . As such, the present invention relates to small RNA compositions and methods for the preparation and use thereof, for example, for agricultural use.

### SUMMARY OF THE INVENTION

The present invention relates to unique small ribonucleic acid molecules, for example siRNAs and miRNAs, identified and isolated using MPSS. Specifically, the invention is directed to the identification of approximately 185,409 unique small RNA sequences from Arabidopsis thaliana (SEQ ID NOS. 1-185,409). In one aspect the invention includes nucleic acids, for example, small RNAs, of from about 15 to about 30 nucleotides in length. In certain preferred embodiments the nucleic acids identified using MPSS are about 17 nucleotides in length. These nucleic acids can be extended with genomic sequence to 21-24 nucleotides in length in order to, for example, determine the entire biologically active or full sequence.

The present invention further relates to a method for genome-scale identification of small RNAs in an organism. Related is the development of a genome-wide library of small RNA sequences of an organism.

Another object of this invention includes the identification of a nucleic acid signature sequence using MPSS that corresponds to at least 15 nucleotides of a small RNA followed by a method for extending such signature sequence to the full length small RNA sequence and/or its mRNA precursor by comparing the signature sequence to a genomic sequence database.

It is a further aspect of the invention to determine, by performing the signature sequence-genomic comparison, one or more discrete locations within the genome where sequence identity is 100%.

Another aspect of the present invention relates to the generation of a library of small RNA molecules identified and/or isolated from an organism. In certain aspects the invention relates to signature sequences and full length small RNA molecules identified and/or isolated from Arabidopsis thaliana. While in other aspects, it is related to a library of signature sequences relating to the small RNAs identified, and/or isolated from an organism.

A specific alternative embodiment of the invention includes a library comprising a plurality of sequences selected from the group consisting of SEQ ID NOs: 1-185,413.

Another embodiment of the present invention includes a small RNA comprising a sequence complementary to a sequence selected from the group consisting of SEQ ID NOs: 1-185,413.

Another embodiment of the present invention includes includes a library comprising a plurality of signature sequences selected from the group consisting of SEQ ID NOs: 1-185,396.

A further aspect of the invention relates to the creation of a database containing, in silico, the sequences of the small RNA molecules identified and/or isolated according to the method of the invention.

Yet another aspect of the present invention relates to the creation of genome-wide small RNA libraries for at least two species, and identifying small RNAs with sequence homology conserved across the species.

It is an additional object of the invention to provide small RNA sequences useful for creating a microarray platform for the identification of differentially regulated small RNAs under any number of conditions.

It is still another object of the invention to provide small RNA sequences useful for “teaching” or training a computer program or algorithm to predict and design small RNA molecules for study or therapeutic applications.

In yet a further object, the invention relates to a vector comprising an RNA sequence and/or transgene that contains at least one recombinant small RNA molecule of the invention. In yet a further object, the invention relates to a vector comprising a DNA sequence and/or transgene that contains recombinant DNA corresponding to a small RNA molecule of the invention. In a related aspect the invention relates to a cell, cell line, or recombinant organism that contains at least one small RNA of the invention, either alone, from its natural precursor and/or in a suitable vector.

In another aspect, the small RNA sequences themselves are useful for performing biological functions, such as for example, RNA interference, gene knockdown or knockout, generating expression mutants, modulating cell growth, differentiation, signaling or a combination thereof for purposes of, for example, experimentation, generating a therapeutic, therapeutic discovery, or generating a novel biological strain. As such, in certain embodiments the invention comprises an isolated small RNA molecule that down-regulates a plant gene, for example, an Arabidopsis thaliana gene, comprising a nucleic acid having at least 75% homology to a member selected from the group consisting of SEQ ID NO. 185,396-185,409 [See Table 13 miR771-miR183], and wherein the nucleic acid is sufficiently complementary to the plant gene to down-regulate the plant gene by RNA interference.

In one embodiment, the invention comprises a small RNA molecule that down-regulates expression of an NBS-LRR disease resistance gene via RNA interference (RNAi). In a preferred embodiment, the small RNA molecule comprises a nucleic acid having at least 75% homology to SEQ ID NO. 185,398.

In another embodiment, the invention comprises a small RNA molecule that down-regulates expression of a DNA (cytosine-5)-methyltransferase gene via RNAi. In a preferred embodiment, the small RNA molecule comprises a nucleic acid having at least 75% homology to SEQ ID NO. 185,399.

In another embodiment, the invention comprises a small RNA molecule that down-regulates expression of an F-box family gene via RNAi. In a preferred embodiment, the small RNA molecule comprises a nucleic acid having at least 75% homology to SEQ ID NO. 185,400.

In another embodiment, the invention comprises a small RNA molecule that down-regulates expression of a galactosidyltransferase gene via RNAi. In a preferred embodiment, the small RNA molecule comprises a nucleic acid having at least 75% homology to SEQ ID NO. 185,401.

In another embodiment, the invention comprises a small RNA molecule that down-regulates expression of a SET domain-containing gene via RNAi. In a preferred embodiment, the small RNA molecule comprises a nucleic acid having at least 75% homology to SEQ ID NO. 185,404.

In another embodiment, the invention comprises a small RNA molecule that down-regulates expression of an S-locus protein kinase gene via RNAi. In a preferred embodiment, the small RNA molecule comprises a nucleic acid having at least 75% homology to SEQ ID NO. 185,405.

In another embodiment, the invention comprises a small RNA molecule that down-regulates expression of an Extra-large G-Protein-related gene via RNAi. In a preferred embodiment, the small RNA molecule comprises a nucleic acid having at least 75% homology to SEQ ID NO. 185,409.

In still another aspect the invention relates to an expression vector comprising a nucleic acid sequence encoding a nucleic acid having at least 75% homology to a member selected from the group consisting of SEQ ID NO. 1-185,409, wherein the expression vector comprises a transcription initiation region; a transcription termination region; and wherein said nucleic acid sequence is operably linked to said initiation region and said termination region. In a preferred embodiment, the expression vector comprises a nucleic acid selected from the group consisting of SEQ ID NO. 185,397-185,409.

These potential uses are given by way of non-limiting example, and are not intended in any way to narrow or limit the scope of the present invention. Other uses will be apparent to those of ordinary skill in the art and are considered as being within the general scope of the present invention.

### DESCRIPTION OF THE DRAWINGS

FIG. 1 provides a small RNA inflorescence library showing numerous chromosomal locations within Arabidopsis of small RNAs.

FIG. 2 shows the plotted distribution of small RNAs disposed across all five Arabidopsis chromosomes.

FIG. 3 depicts the small RNA matching classes of genomic features with categories of genomic features being indicated on the X-axis. Stippled bars indicate the total number of basepairs of the Arabidopsis genome that are found in each category, with the scale indicated on the Y-axis to the right.

FIG. 4 sets forth differential miRNA and siRNA blots, specifically RNA gel blots of low molecular weight RNA isolated from inflorescence tissues (I) and 2-week-old seedlings (S) were probed with labeled oligonucleotides.

FIG. 5 (A)-(B). A five-way Venn diagram of selection criteria for small RNAs. (A) The number of distinct signatures matching the criteria is indicated in each cell; small numbers in upper right corners are used in (B) for additional descriptions.

FIG. 6 (A)-(C). Small RNAs or clusters common to wildtype and rdr2. Venn diagrams representing genome-matched rdr2 454 and MPSS sequences from Table 9. (A) A comparison of distinct signatures in the MPSS libraries indicates 19% of rdr2 sequences were also found in wildtype. (B) A comparison of distinct signatures in the 454 libraries indicates 21% of rdr2 sequences were also found in wildtype. (C) A comparison of genomic clusters of MPSS signatures indicates 93% of small RNA clusters represented in rdr2 were also found in wildtype. For this analysis, clusters contained at least three small RNAs across both libraries; this cutoff was chosen arbitrarily to remove clusters with only one or two small RNAs that could be background. Most of the rdr2-only clusters are low abundance miRNAs or other “real” sequences that were not detected due to depth of coverage in the wildtype library.

FIG. 7. Use of rdr2 sequences to select miRNA candidates from previously identified wildtype small RNAs. Five-way Venn diagram of selection criteria for miRNAs. The number of distinct rdr2 MPSS signatures matching the criteria is indicated in each box numbered in upper right; only rdr2 signatures also found in the wildtype library are represented. The figure excludes 13,153 distinct signatures that did not pass any of the criteria (of which 1,583 were found in both rdr2 and wildtype inflorescence libraries) and 54 matching to the criteria in the Venn which were present in rdr2 but not wildtype inflorescence (these 54 are included in FIG. 12). The paired, sparse, abundance filters, and AtSet1 and AtSet2 filters are described elsewhere but represent potential hairpin structures typical of miRNA precursors, and conservation of those structures in rice, respectively.

FIG. 8. Novel miRNAs identified from Venn analysis of rdr2 sequences. Small RNAs were selected for validation by RNA gel blots, as described in the text. Low molecular weight RNA isolated from inflorescence tissues was probed with labeled oligonucleotides. The lanes in the blots include the following samples: wildtype, rdr2, rdr6, dcl1-7, and dcl2/3/4. The normalized abundance level from the MPSS data for rdr2 and wildtype is listed to the right of the identifier for each small RNA. The reason for the apparent increases in abundance in rdr2 versus wildtype in the blots is not clear; approximately equal amounts of RNA were loaded. It does not appear to be due to RDR2-dependent small RNAs in the 5′ flanking regions, although for miR775, the most extreme case, there is an overlapping small RNA that is largely RDR2-dependent that might interfere with miR775 production in wildtype.

FIG. 9 (A)-(D). Small RNA size distribution in mutants evaluated with 454 sequencing. In each plot, grey indicates wildtype, light blue is rdr2, green is dcl1-7, dark blue is rdr6 and red is the dcl2/3/4 triple mutant. (A) Number of distinct signatures versus size. (B) Total abundance of sequences versus size. (C) Number of distinct versus size, with known miRNAs removed. (D) Total abundance versus size, with known miRNAs removed.

FIG. 10 (A)-(B). RDR2-independent small RNAs from regions with ta-siRNA-like features. (A) The locus that includes small49 exhibits 21-nt phasing and accumulation characteristics in mutants similar to those of ta-siRNAs. Image of the MPSS web viewer for the intergenic region that contains small49 in the position indicated; an RNA gel blot of small49; a plot of the Y-axis indicating the small RNA abundance in the rdr2 mutant as measured by MPSS (in TPQ) and the X-axis indicating nucleotide position on Chr. 1, with the “697” indicating position 25,282,697. (B) The blot shows that small58 also has ta-siRNA-like accumulation features; images as described in (A), with the 0 position in the X-axis of the plot indicating nucleotide 13,295,900 on Chr. 4.

FIG. 11 (A)-(C). Comparison of MPSS and 454 sequence data for rdr2. Venn diagram representing genome-matched rdr2 454 and MPSS sequences from Table 9. To compare the different length 454 and MPSS sequences for the center of the Venn diagram, 454 signatures were counted if an MPSS signature was contained anywhere within the sequence. Because the MPSS signatures are shorter, some match to more than one 454 sequence. “wt” indicates wildtype. (B) Abundance plot for rdr2 454 and MPSS data (genome-matched sequences only). The dotplots indicate the correlation among abundance levels for genome-matching sequences identified by both technologies for both wildtype (wt, on left) and rdr2 (on right). In order to visualize the distribution at the lower expression levels, a small number of higher abundance data points are not shown. The abundance of each distinct 17 nt MPSS signature was compared to the sum of abundance of all 454 sequences with the same first 17 nt. (C) Histograms illustrate the number of distinct sequences in each technology for both wildtype and rdr2 inflorescence libraries. For the two plots of MPSS data, the X-axis indicates a range of the normalized abundance for the distinct signatures (TPQ), whereas for the two 454 plots, the X-axis represents raw values. Compared to the corresponding wildtype libraries, a higher proportion of small RNAs were sequenced multiple times from rdr2 with both 454 and MPSS indicating that the rdr2 sequencing is closer to saturation.

FIG. 12 (A)-(B) Distribution of rdr2 and wildtype small RNAs among different genomic features. Histograms of matches to genomic features for wildtype and rdr2 MPSS libraries. Wildtype data is indicated by grey bars, rdr2 data is indicated by black bars. These data are enumerated in Table 11. (A) The number of distinct signatures corresponding to each class of genomic feature. (B) The sum of the abundances (in TPQ) corresponding to the distinct signatures in each class of genomic feature.

FIG. 13 (A)-(I) Potential secondary structures of new miRNA precursors. Secondary structures were predicted for the nine new miRNAs. These structures were predicted using mFOLD (http://www.bioinfo.rpi.edu/applications/mfold/). The miRNA sequences identified by MPSS analysis are indicated with curly braces. The RNA gel blots for these small RNAs are shown in FIG. 8. (A) Genomic region encoding miR771. The region is on Chr. 3 between AT3G53010 and AT3G53020. (B) Genomic region encoding miR772. The region is on Chr.1 between AT1G12290 and AT1G12300. (C) Genomic region encoding miR773. The region is on Chr.1 between AT1G35500 and AT1G35510. (D) Genomic region encoding miR774. The region is on Chr.1 between AT1G60070 and AT1G60075. (E) Genomic region encoding miR775. The region is on Chr. 1 between AT1G78200 and AT1G78210. (F) Genomic region encoding miR776. The region is on Chr. 1 between AT1G61730 and AT1G61740. (G) Genomic region encoding miR777. The region is on Chr. 1 between AT1G70640 and AT1G70650. (H) Genomic region encoding miR778. The region is on Chr. 2 between AT2G41610 and AT2G41620. (I) Genomic region encoding miR779. The region is on Chr. 2 between AT2G22490 and AT2G22500.

FIG. 14 (A)-(D). Predicted targets of new miRNAs. Targets were predicted using the method described by Jones-Rhoades and Bartel (2004). The mRNA target is shown above and the miRNA below in each alignment; matches are indicated with vertical lines, mismatches are unmarked and G-U wobbles are indicated with a circle; grey text indicates nucleotides flanking the target site; for experimentally validated targets, the arrow indicates a site verified by 5′ RACE, with the number of cloned RACE products sequenced shown above. In this algorithm, each mismatch is given a score of 1, each wobble (G:U mismatch) is given a score of 0.5, and each bulge is given a score of 2. Only targets with a penalty score of less than or equal to 1.5 are shown in this figure; a complete list of targets scoring 2.5 or less is shown in Table 13.

FIG. 15. Foldback sequences are sources of numerous rdr2-independent small RNAs. Inverted repeats are predicted to form “foldback” hairpin structures that are the source of numerous small RNAs in the rdr2 libraries. Although the difference in the length of the repeat unit is statistically significant between the RDR2-dependent and RDR2-independent sets, some RDR2-independent inverted repeats are quite short (see lower examples). This figure shows views from our website; small RNAs are black triangles, inverted repeats are orange shaded regions. Open triangles indicate a match to more than one location in the genome; most small RNAs in these inverted repeats match twice, once in each arm of the repeat. Small57 may be an evolving miRNA locus. This locus is the same as ASRP1729

FIG. 16 (A)-(C). The A. thaliana gene encoding SRK contains an inverted repeat that is the source of RDR2-independent small RNAs. (A) An image of the A. thaliana SRK locus, with the inverted repeat shown in orange, exons of SRK (At4g21370) indicated as blue boxes, and the annotated adjacent gene (At4g21366) shown in red. (B) An RNA gel blot of small85 from the SRK locus. (C) A total of 963 nt of sequence from the inverted repeat spanning At4g21370 and At4g21366 was analyzed using mFold. This sequence is predicted to form a near-perfect double-stranded RNA of 390 bp. Small RNAs were identified by MPSS that matched throughout the stem structure but were absent from the loops

FIG. 17 Enrichment of small RNAs at the TAS1a locus in rdr2 compared to wildtype. Bars indicate the abundance of the small RNAs (MPSS data, in TPQ) found at each position within the locus; bars above the center line indicate the upper strand, bars below the center line indicate the bottom strand. Red bars indicate small RNAs in wildtype and black bars indicate small RNAs in rdr2. Due to limited space, non-expressed sites have been removed. The upper and lower boxes are in logarithmic scale to indicate the most abundant small RNAs. The position within the locus is indicated near the bottom, with the zero position indicating the functional ta-siRNA which is identified by the MPSS signature TTCTMGTCCMCATAG found at 6169 TPQ in rdr2, corresponding to Ser. No. 11,729,063 bp on Chr. 2.

FIG. 18 Correlation of miRNA gene abundances in the rdr2 and the dcl2/3/4 triple mutant. The figure is based on the 454 data for these mutant lines shown in Table 10. Due to the plot scale and its abundance, miR172 is not shown. The diagonal line indicates the trend line for the data. The high-abundance miRNA genes are marked for reference. X- and Y-axis values are raw abundances.

FIG. 19 contains Table 1 from Example 1.

FIG. 20 contains Table 2 from Example 1.

FIG. 21 contains Table 3 from Example 1.

### DESCRIPTION OF THE INVENTION

As used herein, the term “small RNA” refers to those RNA molecules that are larger than about 10 nucleic acids in length but less than about 50 nucleotides, and is used generally to refer to siRNAs, miRNAs, and other small or tiny RNAs. Small RNAs may be produced in an intact form or following processing from a larger molecule. Small RNA molecules are generally “noncoding” and exert their function as RNAs.

As used herein, the term “nucleic acid” is used in a general sense to refer at least one of ribonucleic acid (RNA), ribonucleotide, deoxyribonucleic acid (DNA), deoxyribonucleotide, nucleic acid analog, synthetic nucleotide analogs, nucleic acid conjugates, for example peptide nucleic acids or locked nucleic acids, nucleic acid derivatives, polymeric forms thereof, and includes either single- or double-stranded forms. Also, unless expressly limited, the term “nucleic acid” includes known analogues of natural nucleotides that have similar binding properties as the reference nucleic acid. In addition, a particular nucleotide or nucleic acid sequence includes conservative variations based on the nucleotides adenine (“A”), guanine (“G”), cytosine (“C”), thymine (“T”), uracil (“U”), and inosine (“I”).

Previously we presented a method for the isolation of small RNA from Arabidopsis. (U.S. application Ser. No. 11/204,903) This method allowed for an increase in the number of distinct small RNA sequences known by more than an order of magnitude. The present invention relates generally to the isolation and identification of small ribonucleic acids (RNAs), for example, small inhibitory RNAs (siRNAs), microRNAs (miRNAs), tiny RNAs or combinations thereof from an organism using the process disclosed in the above patent applications. The present invention is directed to identification of small RNAs from the flowering plant Arabidopsis thaliana. We have identified approximately 185,396 unique nucleic acid signature sequences (SEQ ID NOS. 1-185,396) from Arabidopsis thaliana.

In a preferred embodiment, SEQ ID NOS 1-185,396 are referred to as signature sequences. Generally, these signature sequences do not always correspond to the full length, endogenously or biologically functional small RNA sequence. In a preferred embodiment, the present invention relates to a method for determining the full length small RNA sequence and/or its mRNA precursor by comparing the signature sequence, for example a 17-mer, to a high quality genomic sequence database, for example by BLAST or other sequence comparing algorithm. By performing the signature sequence-genomic comparison, one or more discrete locations within the genome can be identified where sequence identity is 100%. The full length small RNA can therefore be determined by extending the 17-mer signature sequence in either the 5′ or 3′ direction upon which direction the molecule is sequenced from. In certain aspects of this embodiment, the signature sequence is extended in the 3′ direction for a suitable number of nucleotides. More particularly, the signature sequence is extended in the 3′ direction by from about 1 to about 13 bases. It is generally accepted that the major type of siRNAs (chromatin siRNAs) in plants are about 24 nucleotides, and miRNAs are typically about 21 nucleotides in length. Therefore, in a particularly preferred embodiment the 17 nucleotide signature sequence would be extended about 7 bases in the case of a siRNA, or about 4 bases in the case of a miRNA. However, one of ordinary skill in the art will recognize that the precise number of nucleotides selected to extend the signature sequence to a full length small RNA will depend on a number of considerations, such as for example, whether the small RNA appears to be a siRNA or a miRNA, whether the small RNA appears to be located within a cluster, and the like.

A method of extending the signature sequences identified using MPSS to their full functional length through the use of a high quality genomic database for the organism of interest is preferably used. Generally stated, the method comprises the steps of: (a) providing a high quality genomic DNA database; (b) providing identification of small RNA signature sequences of from about 15 to about 20 nucleotides in length; (c) comparing the small RNA signature sequences to the genomic database, for example, by using a string (text)-searching program or a sequence identity algorithm such as BLAST; (d) identifying the genomic regions that indicate identity with the signature sequence; and (e) extending the signature sequence in the 3′ direction by from 1 to about 13 nucleotides to obtain the full sequence of the biologically active molecule. This method allows for the identification of the full length small RNA or the small RNA source or precursor without performing tedious cloning steps that are not sensitive enough to clone the majority of low abundance small RNAs.

In a preferred embodiment the present invention encompasses nucleic acid molecules, for example, single or double stranded small RNAs, siRNAs, miRNAs, tiny RNAs, analogs, precursor molecules of DNA or RNA, and combinations thereof, isolated from the plant, Arabidopsis thaliana, that are associated with physiological regulatory mechanisms. In yet another of the preferred embodiments, the small RNAs of the present invention preferably have a length of from about 15 to about 30 nucleotides, but may be provided as a precursor with a length of from about 16-100 nucleotides.

In a particular preferred embodiment, the present invention relates to the small RNAs SEQ ID NOS 1-185,413, and sequences containing at least about 75% homology to those sequences. The present invention also relates to any sequence having the same biological activity as any of SEQ ID NOS 1-185,413, and, alternatively, covers any sequence that is adjacent to or overlaps the target site by at least about 75% homology. In another of the preferred embodiments the present invention encompasses nucleic acid sequences which hybridize under stringent conditions with the nucleic acid sequences listed in SEQ ID NOS 1-185,413.

In another of the preferred embodiments the invention encompasses a nucleic acid molecule that contains at least one modified nucleic acid or non-naturally occurring nucleotide analog. It is contemplated that the modified or non-naturally occurring nucleic acid or nucleotide analog may be placed anywhere along the length of the sequence, for example, at the 5′-end, or the 3′end.

In still another preferred embodiment the present invention encompasses a recombinant expression or cloning vector, for example a bacterial plasmid-derived vector, or viral vector, comprising a small RNA molecule of the invention, SEQ. ID: 1-185,413. The vector may be an RNA or DNA vector adapted for use in a suitable system or organism, or a combination thereof under suitable conditions. The vector preferably results in the transcription of the small RNA molecule or cluster of small RNA molecules as such, a precursor or primary transcript thereof, which is further processed to the desired small RNA molecule. A “cluster” refers to more than one small RNA that match to nearby genomic sequences. In an aspect of this embodiment, the small RNAs of the invention may be delivered by any suitable means known to those in the art, including for example, T-DNA mediated transformation, particle bombardment, electroporation, receptor-mediated gene therapy, recombinant virus gene therapy, liposome mediated gene transfer, calcium phosphate mediated gene transfer, polyamine conjugated nucleic acid gene transfer, and the like.

In still another aspect the invention relates to an expression vector comprising a nucleic acid sequence encoding a nucleic acid having at least 75% homology to a member selected from the group consisting of SEQ ID NO. 1-185,413, wherein the expression vector comprises a transcription initiation region; a transcription termination region; and wherein said nucleic acid sequence is operably linked to said initiation region and said termination region. In a preferred embodiment, the expression vector comprises a nucleic acid selected from the group consisting of SEQ ID NO. 185,397-185,413.

The invention is further directed to the development of a library of small RNAs from a particular organism comprising a plurality of sequences identified using the method of the invention. In a preferred embodiment, the library consists of virtually all small RNA sequences of a particular organism, or at least all of those small RNA sequences that are consistently expressed throughout all tissues of said organism. It is contemplated herein that SEQ ID NOs: 1-185,396 are the signature sequences for the small RNA sequences of the organism Arabidopsis thaliana that are most consistently expressed throughout the tissues of this plant. In a preferred embodiment, therefore, the invention relates to a library consisting of a plurality of small RNA sequences selected from SEQ ID NOs: 1-185,396. The invention is further directed to a library consisting of the full length sequences identified from SEQ ID NOs: 1-185,396. Alternatively stated, the invention is directed to the creation of a database containing, in silico, the sequences of the small RNA molecules identified and isolated according to the method of the invention.

The invention is also directed to the isolation and identification of individual full length small RNA molecules from Arabidopsis thaliana. Upon such identification, biological function of the small RNA molecule can be tested using a variety of methods known in the art. Once biological activity of a small RNA has been identified, specific functional aspects of the organism can be purposefully addressed. For example, contemplated herein is a method of changing or introducing a phenotypic trait of an organism by increasing or decreasing the function or level of one or more small RNAs, which impact their ability to silence target genes or regions of the genome they target. In a related embodiment the invention includes a method for performing RNA interference (RNAi) comprising the delivery of an effective amount of at least one small RNA sequence of the invention, in a suitable form that results in gene knockdown, knock-up, or knockout. In other related embodiments, multiple small RNAs of the invention may be delivered, for example a siRNA cluster, to affect a gene, family of genes, or signaling pathway that results in an altered trait. Some specific aspects of this embodiment include, for example overproduction of a small RNA to make plants more resistant to salt stress comprising the steps of (a) selecting a small RNA randomly or based on a characteristic, for example, being induced when plants are treated with the plant hormone ABA that controls responses to salt and other stresses; (b) overproducing the small RNA resulting in plants to create salt-resistant traits. Another example would include modulation of the expression of certain genes in a plant that would affect its tolerance to pesticides, temperatures or soil condition.

More detailed examples of this embodiment include use of a small RNA of the invention that could identify a small RNA source gene that could in turn be inactivated to accomplish the control of a process such as the control of nutrient uptake or content. The term “nutrient uptake” is intended to describe nutrient uptake that helps the plant grow more efficiently or in difficult growing conditions, for example. The term “nutrient content” is intended to describe the nutrients produced in the plant, such as, for example, lysine, vitamin A, vitamin C, etc. This method comprises, (a) predicting targets of the small RNA that may silence nutrient genes involved in the uptake of nutrients or production of genes that would affect nutrient content; (b) choosing such a small RNA and identify insertion mutants from public collections that have insertions in the source gene or near the DNA (genomic match) for the small RNA; and (c) testing if these mutants have altered or improved nutrient uptake or content.

In yet another example of this embodiment, a small RNA of the invention can be used to create a therapeutic or viral resistance trait using knowledge from natural small RNAs. This method comprises, (a) using small RNA sequence characteristics (e.g. siRNA sequences) to refine computer programs currently used to design dsRNA sequences to be used for RNAi against the RNA from for example, a harmful virus or other plant pathogen such as bacterial, fungal, nematode, or parasitic plant; (b) building a dsRNA gene that in the plant will make small RNA with optimized design that will be complementary to the virus or other pathogen RNA; and (c) introducing this gene into the plant to test if it works better to control viral or pathogen infection than others designed without using the natural small RNAs to train the computer program.

In certain embodiments, the invention relates to the use of the full length small RNA sequences of the invention themselves are useful for performing biological functions, such as for example, RNA interference, gene knockdown or knockout, generating expression mutants, modulating cell growth, differentiation, signaling or a combination thereof for purposes of, for example, experimentation, generating a therapeutic, therapeutic discovery, or generating a novel biological strain. As such, in certain embodiments the invention comprises an isolated small RNA molecule that down-regulates a plant gene, for example, an Arabidopsis thaliana gene, comprising a nucleic acid having at least 75% homology to a member selected from the group consisting of SEQ ID NO. 185,397-185,409 [See Table 13], and wherein the nucleic acid is sufficiently complementary to the plant gene to down-regulate the plant gene by RNA interference.

In one embodiment, the invention comprises a small RNA molecule that down-regulates expression of an NBS-LRR disease resistance gene via RNA interference (RNAi). In a preferred embodiment, the small RNA molecule comprises a nucleic acid having at least 75% homology to SEQ ID NO. 185,398.

In another embodiment, the invention comprises a small RNA molecule that down-regulates expression of a DNA (cytosine-5)-methyltransferase gene via RNAi. In a preferred embodiment, the small RNA molecule comprises a nucleic acid having at least 75% homology to SEQ ID NO. 185,399.

In another embodiment, the invention comprises a small RNA molecule that down-regulates expression of an F-box family gene via RNAi. In a preferred embodiment, the small RNA molecule comprises a nucleic acid having at least 75% homology to SEQ ID NO. 185,400.

In another embodiment, the invention comprises a small RNA molecule that down-regulates expression of a galactosidyltransferase gene via RNAi. In a preferred embodiment, the small RNA molecule comprises a nucleic acid having at least 75% homology to SEQ ID NO. 185,401.

In another embodiment, the invention comprises a small RNA molecule that down-regulates expression of a SET domain-containing gene via RNAi. In a preferred embodiment, the small RNA molecule comprises a nucleic acid having at least 75% homology to SEQ ID NO. 185,404.

In another embodiment, the invention comprises a small RNA molecule that down-regulates expression of an S-locus protein kinase gene via RNAi. In a preferred embodiment, the small RNA molecule comprises a nucleic acid having at least 75% homology to SEQ ID NO. 185,405.

In another embodiment, the invention comprises a small RNA molecule that down-regulates expression of an Extra-large G-Protein-related gene via RNAi. In a preferred embodiment, the small RNA molecule comprises a nucleic acid having at least 75% homology to SEQ ID NO. 185,409.

In yet another embodiment, the small RNAs of the invention can be used in a method of performing cross-species analysis of small RNAs. This method includes taking one or more of the small RNA SEQ ID NOS 1-185,413, from Arabidopsis thaliana, and performing a sequence identity comparison, for example, using BLAST analysis, with a genomic-wide library of small RNA isolated from another species, for example, another eukaryote such as another plant species, fungi, yeast or a mammal, and isolating those small RNAs that display conservation over at least part of the small RNA sequence. In a related embodiment, the invention comprises taking one or more of the small RNA SEQ ID NOS 1-185,413, from Arabidopsis thaliana, and performing a sequence identity comparison, for example using BLAST analysis, with a genomic library from another species, for example, another eukaryote such as another plant species, fungi, yeast, or mammal, and identifying those small RNAs that display conservation over at least part of the small RNA sequence. Generally, a nucleotide sequence demonstrating at least 30% homology is considered homologous. This can provide useful information about target genes, small RNA precursors, as well as small RNA regulation and control over phenotypic traits. Several algorithms have been proposed for performing this analysis, such as by Rhoades, M., et al., 2002, Prediction of Plant MicroRNA Targets. Cell 110: 513-520; Lewis B P, et al. Prediction of Mammalian MicroRNA Targets. Cell 2003, 115:787-798; and Wang, X, et al., 2004, Prediction and Identification of Arabidopsis thaliana MicroRNAs and their mRNA Targets. Genome Biol 5: R65, which are incorporated herein by reference in their entirety. In a related aspect, one or more small RNA sequences of SEQ ID NOS. 1-185,413 can be used to generate a database useful for comparison with small RNA from other plant species isolated under varying conditions, other developmental states, other organisms or the like. In still another related aspect, one or more small RNA sequences, of SEQ ID NOS. 1-185,413 comprise a microarray, for example a DNA chip, to allow for high-throughput analysis of differential regulation of the small RNAs in the library.

In certain embodiments the small RNAs of the invention can be useful for experimental or therapeutic applications. For example, quantitative measurements of small RNA sequences identified according to this method would be useful for understanding processes such as cell differentiation, gene expression, cell signaling responses and pathways, and disease state cell processes.

Alternatively, identified small RNAs can be useful for determining genes and RNA molecules that are critical for development, growth, and maintenance of an organism by identifying small RNA molecules that have been evolutionarily conserved across species. For instance, genome-wide small RNA libraries could be created for at least two species, and small RNAs with sequence homology conserved across the species can be identified. In certain instances, the small RNAs can be used to identify those molecules unique to a species. In other instances the small RNAs of the invention can be used to predict the endogenous mRNA or noncoding RNA targets of miRNAs or other trans-acting small RNAs such as siRNAs. Basic strategies and algorithms for performing these predictions have been published by Rhoades, M., et al., 2002, Prediction of Plant MicroRNA Targets. Cell 110: 513-520; Lewis B P, et al. Prediction of Mammalian MicroRNA Targets. Cell 2003, 115:787-798; and Wang, X, et al., 2004, Prediction and Identification of Arabidopsis thaliana MicroRNAs and their mRNA Targets. Genome Biol 5: R65, which are incorporated herein by reference in their entirety.

In certain aspects of the preferred embodiments miRNA targets can be found with the assistance of computer algorithms designed for that, or by looking at the RNA levels for all genes of an organism, for example Arabidopsis, with DNA microarrays, and sequence comparisons for regions complementary to the small RNAs. In other aspects of this embodiment, siRNA targets are determined by identifying the siRNA source, because often times the siRNAs cause the corresponding DNA to be silenced at the chromatin level by methylation. Targets can be identified with sequences having as low as 75% homology to SEQ ID NOS. 1-185,413 in accordance with the rules for mismatch analysis, etc. as described in the references above. In some aspects, the small RNAs identified can be used to identify genomic sequences with perfect or near perfect matches that are targeted for chromatin modification or other forms of regulation by the small RNAs. Alternatively, the creation of an in silico series of variants of the natural small RNAs could be used to create variant small RNA genes with different target specificity, whilst preserving the flanking sequences such as hairpin-like structures.

Other embodiments include small RNA sequences that can be used to create a microarray platform, for example, nucleic acid “chips,” polymeric microspheres or beads, and the like for the identification of differentially regulated small RNAs under any number of conditions, for example, treatment with a chemical compound, developmental stage, disease condition, and the like. In related embodiments, small RNA sequences can be used for “teaching” or training a computer program or algorithm to predict and design small RNA molecules for study or therapeutic applications. The small RNA sequences can also provide information that can be used to design better double-stranded RNA for RNAi strategies.

In alternate embodiments, a small RNA sequence and/or transgene that contains at least one recombinant small RNA molecule can be incorporated into a vector. The vector may be, for example, a plasmid vector or a bacterial vector or a viral vector, as an RNA or DNA molecule or modified RNA molecule suitable for expression or function in a particular cell, for example, a prokaryotic cell, a eukaryotic cell, a primary cell, or a cell line. Relatedly, the invention relates to a cell, cell line, or recombinant organism that contains at least one small RNA of the invention, either alone, from its natural precursor and/or in a suitable vector.

The small RNA sequences themselves can also be useful for performing biological functions, such as for example, RNA interference, gene knockdown or knockout, generating expression mutants, modulating cell growth, differentiation, signaling or a combination thereof for purposes of, for example, experimentation, generating a therapeutic, therapeutic discovery, or generating a novel biological strain. As described earlier, the small RNAs can be used to change or introduce phenotypic traits by increasing or decreasing the function or level of one or more small RNAs, which impact their ability to silence target genes or regions of the genome they target. In some cases, multiple small RNAs, for example, a cluster of siRNAs, might be used at one time to regulate one or more targets to create a desired or advantageous trait. As such, the present invention also relates to a transgene or vector comprising, encoding, or facilitating the production of multiple small RNAs or a small RNA cluster.

In another of the preferred embodiments, the small RNAs of the invention, SEQ ID NOS 1-185,413 comprise a “teaching” set of sequences for a computer algorithm to improve and enhance in silico design and prediction confidences of small RNAs, their genes, or precursors. In addition, a library of the small RNAs of the invention can be used to design algorithms that are better able to predict and design sequences for use in RNAi.

In yet another embodiment, the invention includes a kit comprising one or more small RNAs of the invention. In a preferred embodiment, the kit includes a library of small RNAs. The invention also relates to the diagnostic, trait improvement, such as crop improvement, therapeutic, or prophylactic use of the small RNA sequences. For example, detection of any one of the small RNAs of SEQ ID: 1-185,413 may be used to determine or classify a particular condition, classify a cell or tissue type, or developmental stage.

In another embodiment of the present invention the small RNA of the invention may be used as starting materials for the manufacture of sequence-modified small RNA molecules, which may contain nucleic acid modifications in order to modify the target-specificity of the small RNA.

It will be understood by those of ordinary skill that the compositions of the present invention may be used in any suitable form, for example, a solution, a spray, a powder, an injectable solution, an ointment, tablet, suspension, emulsion, and the like; combined with any suitable carrier that increases the stability, facilitates uptake or both, for example, a liposome, a cation, and the like; or administered in any suitable way, for example, by transfection, infection, injection, or topical delivery.

It is understood that the examples and embodiments described herein are for illustrative purposes only and that various modifications or changes in light thereof will be suggested to persons skilled in the art and are included within the spirit and purview of this application and are considered within the scope of the appended claims. All publications, patents, and patent applications cited herein are hereby incorporated by reference in their entirety for all purposes.

As will be understood by one of ordinary skill in the art, the techniques described and hereby incorporated into the present invention are generally applicable and may be varied in any number of ways without departing from the general scope of the invention. Also, additional advantages and features of the present invention will be recognized by those of skill in the art in view of the description and the following examples. The examples provided herein are provided for illustrative purposes only and are in no way considered to be limiting to the present invention. For example, the relative quantities of the ingredients may be varied to achieve different desired effects, additional ingredients may be added, and/or similar ingredients may be substituted for one or more of the ingredients described.

### EXAMPLE 1

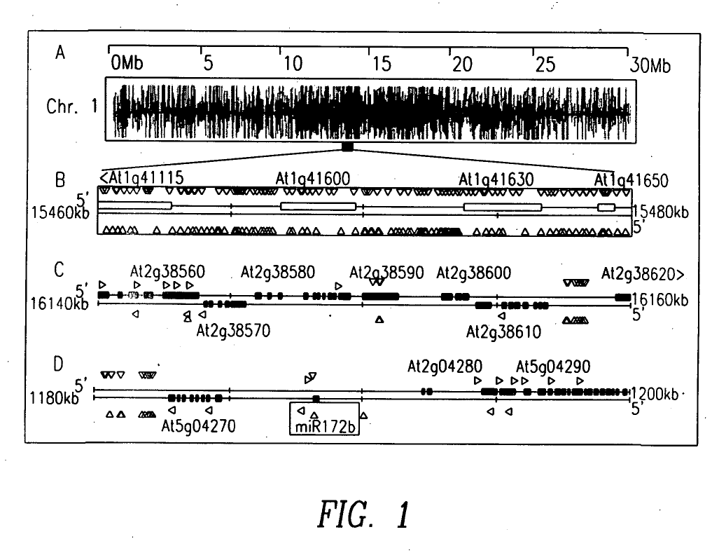

Adaptation of MPSS for small RNA analysis. To investigate the full complexity of small RNAs, we modified and customized the MPSS vectors and procedures to adapt the MPSS methodology for the sequencing of these molecules. We sought to take advantage of the power of MPSS to sequence hundreds of thousands of molecules per sequencing run. Prior applications of MPSS made use of the poly(A) tail of mRNAs to facilitate cDNA synthesis and sequenced only molecules with a 5′ terminal sequence of ‘GATC’ or ‘CATG’, generated by a restriction enzyme like DpnII or NlaIII. Because most small RNAs are unlikely to begin with these restriction sites or contain a poly(A), the MPSS cloning vectors were adapted to initiate sequencing from the first nucleotide, regardless of the sequence. An overview of the method is shown in Supplementary FIG. 1. Briefly, small RNA molecules are isolated by size fractionation on a polyacrylamide gel, RNA adapters are sequentially ligated to the 5′ and 3′ ends, and reverse transcriptase is used to generate the first strand of cDNA which is amplified and used as the template for MPSS. Shown in FIG. 1 is a small RNAs map to numerous chromosomal locations. (A) Shows the small RNAs from the inflorescence library arrayed on Arabidopsis chromosome 1. Vertical lines indicate the location and abundance of a small RNA on the top or bottom strand. The height of the vertical lines indicates the abundance of the small RNA, with the maximum height indicating >25 transcripts per quarter million (TPQ) and red bars indicating >125 TPQ. (B) shows a pericentromeric region from Chr. 1, in which the Arabidopsis small RNAs are shown as black triangles above or below the double-stranded chromosomes. The red or blue boxes indicate exons on top or bottom strands, respectively. Colored triangles indicate the location of mRNA MPSS signatures. Hollow triangles indicate signatures mapping to more than one location in the genome. Retrotransposon-related sequences identified by RepeatMasker are highlighted in pink, and this entire region was found to be repetitive, including spaces between annotated retrotransposons indicated as thin yellow bars. (C) shows a typical genic region; most small RNAs map to intergenic regions which are often unannotated transposon-related sequences (yellow shading indicates DNA transposon-related sequences identified by RepeatMasker). (D) shows an intergenic region of Chr. 5; the orange box indicates small RNAs and mRNA MPSS signatures that correspond to mir172.

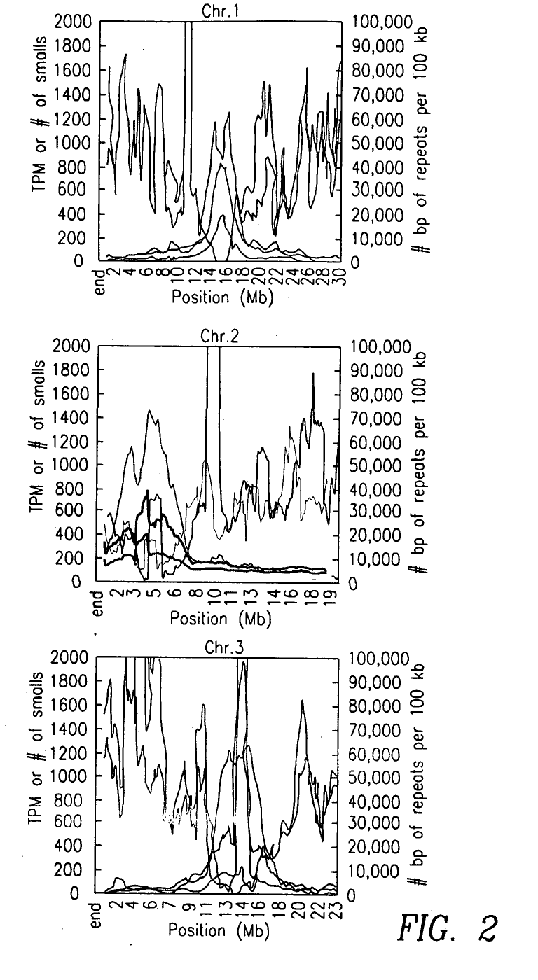

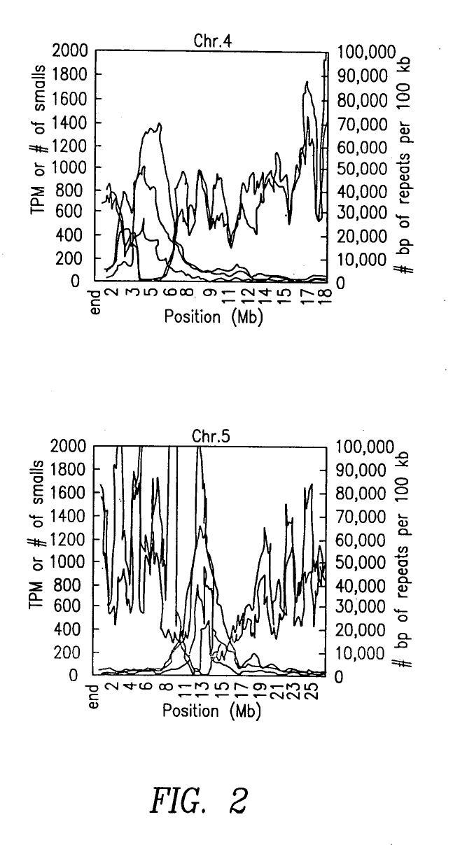

Genome-wide analysis of small RNAs in Arabidopsis. Pericentromeric heterochromatin is known to be a rich source of small RNAs due to a high concentration of transposable elements. We examined the distribution of small RNAs on the five Arabidopsis chromosomes and we compared this distribution to that of repeats and mRNA abundance data (FIGS. 1A, 1B, and 2). The small RNAs from both libraries were highly concentrated in the pericentromeric regions of each chromosome, but matches could be found throughout the length of the chromosomes. In contrast, mRNA levels, as detected by MPSS analysis of similar tissues, were greatest in the euchromatic regions (FIG. 2). FIG. 2 shows the distribution of small RNAs across Arabidopsis chromosomes. The five Arabidopsis chromosomes are indicated in panels A to E. Distributions were plotted as a moving average of 10 adjacent bins of 100 kb genomic sequence. The x-axis indicates the position on each chromosome in megabases. For the 100 kb bins, the left y-axis indicates either the average number of matching small RNA signatures or the sum of the abundance of mRNA MPSS signatures (in transcripts per million, TPM) (23). The right y-axis indicates the number of nucleotides identified as a repeat by RepeatMasker in each 100 kb bin (green lines). The average number of matching small RNAs was calculated across the chromosomes from the inflorescence (dark blue lines) and seedling (red lines) libraries, respectively. Relative transcription of mRNA was measured by MPSS on mRNA from inflorescence (thin blue lines) and seedling (thin black lines) libraries; these libraries were produced for unrelated experiments with slightly different growth conditions (see Materials and Methods available as supporting material). The boundaries of the pericentromeric regions are delineated by the points at which the repeats exceed approximately 20,000 bp per 100 kb. Repeats and small RNAs co-localized to the pericentromeric heterochromatic regions, as illustrated by the extensive coverage of such sequences by small RNAs in a representative region of Chromosome 1 (FIG. 1B). Although FIGS. 1B, 1C and 1D show views from our web site, the small RNA data for specific genomic locations, including the examples we describe, are best examined and interpreted by using the website (http://mpss.udel.edu/); the site provides detailed information about each signature that can be accessed by clicking on the corresponding triangle.

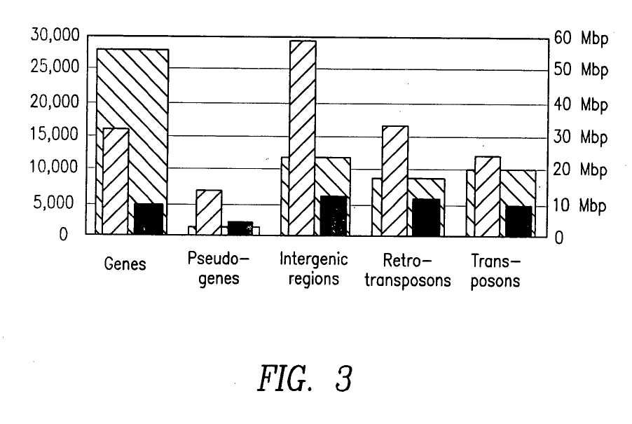

Table 1 (See FIG. 19) show the genomic localization of small RNA signatures and clusters. Indeed, more than half of the genomic sequences matching the small RNAs in the two libraries were transposons or retrotransposons (Table 1A). The corresponding small RNA signatures were predominantly found at moderate abundances (11 to 100 TPQ, transcripts per quarter million). However, they represented less than half the number of distinct small RNAs (FIG. 3) because more than 80% of these predicted siRNAs were matched multiple locations in the genome. The small proportion of single-site matches apparently target specific mobile elements or unique regions of such elements. In each library, at least two-thirds of the total set of transposon-related sequences in the Arabidopsis genome had matches to small RNAs. Similarly, small RNAs matched to 66% of the total annotated pseudogenes (Table 1A) and these RNAs were of moderate abundance and had multiple matches in the genome. On a per megabase basis, pseudogenes matched the greatest number of small RNAs in both libraries, suggesting that these sequences are the subject of substantial RNA-mediated gene silencing (FIG. 3). Specifically, FIG. 3 depicts the small RNA matching classes of genomic features with categories of genomic features being indicated on the X-axis. Stippled bars indicate the total number of basepairs of the Arabidopsis genome that are found in each category, with the scale indicated on the Y-axis to the right. Retrotransposon and transposon categories are based on RepeatMasker results. Within each category, the grey vertical bar indicates the total number of distinct small RNAs matched from the inflorescence library and the black vertical bar indicates the total number of distinct small RNAs matched from the seedling library; the scale for distinct small RNAs is indicated on the Y-axis to the left.

The relative number of distinct small RNAs per megabase of sequence was lower for genes than for any other genomic sequence (FIG. 3; Tables 1A and B). Only 7% of annotated genes had matches in the seedling library and more than twice this number in the inflorescence library, and in both cases, approximately two-thirds of the genes that were matched had relatively few small RNAs (1 to 10 TPQ). These low-abundance signatures could represent perfectly matched miRNAs, or siRNAs targeted to silenced genes, unannotated pseudogenes, unannotated repeats, or other unknown sources of siRNAs (FIG. 1C). In Table 2 (See FIG. 20), the classification of genes perfectly matched by small RNA clusters is shown. We determined the number of genes in different GO functional categories matched by small RNAs to address whether any functional class of genes was over-represented (Table 2). The small RNAs were well distributed among the broad range of cellular processes and molecular functions; reflecting the diversity of small RNAs, approximately half as many genes matched in seedlings as in inflorescence. To assess whether the small RNAs that match to genes could be derived from degradation products of longer mRNAs, we compared mRNA and small RNA MPSS data for highly expressed genes. Highly expressed genes like rubisco subunits and chlorophyll a/b binding protein matched small RNAs that comprised less than 0.1% of the total, suggesting a low rate of contamination by degradation products (data not shown).

The number of distinct small RNAs that matched to intergenic regions exceeded the numbers that matched to genes, pseudogenes, transposons, or retrotransposons (FIG. 3), an observation that cannot be explained by the fraction of the genome that these entities comprise. The diversity of intergenic small RNAs was approximately four-fold greater in inflorescence than seedling (FIG. 3; Table 1A and B). Small RNAs in the intergenic regions potentially represent either miRNAs or siRNAs from unannotated repeats. Some intergenic small RNAs could be derived from tandem or inverted genomic repeats; measured across the genome, we observed a good correlation between the quality of the repeat and the number of small RNAs (tandem repeats, R=0.5986; inverted repeats, R=0.4955). These analyses also demonstrated that the inflorescence small RNAs were consistently at least three-fold more complex than seedling small RNAs, with the most pronounced difference in complexity in the intergenic regions (FIG. 3).

Previous studies have demonstrated that miRNAs monitored with “sensor” transgenes can lead to the production of secondary siRNAs that match the sensor mRNA outside the sequence originally targeted. This production of secondary siRNAs is known as transitivity. We examined 61 known or predicted targets of Arabidopsis miRNAs for evidence of transitivity. Only four targets (At1 g62670, At1 g63080, At1 g63150, and At1 g63400), all of which encode pentatricopeptide (PPR) repeat-containing proteins, matched substantial numbers of small RNAs, and these were primarily in repeated regions within each gene. Most targets had no matching small RNAs other than miRNAs, or the only matching small RNAs were few, of very low abundance, or corresponded to repeats. This indicates that transitivity by miRNAs is of little biological significance in seedlings or inflorescence and is likely a transgene phenomenon as hypothesized previously.

One of the characteristics of siRNAs is that multiple siRNAs are cleaved from the same dsRNA precursor, and these can derive from either strand. Thus, the population of precursors from a given region leads to the production of numerous siRNAs that will be particularly abundant for repetitive sequences if the repeats are all sources of siRNAs. Despite the 21-24 nucleotide size of these small RNAs, the presumably stochastic nature of this process is unlikely to lead to regular pattern or periodicity in most genomic regions; we saw no evidence of a regular 21 to 24 nucleotide pattern of small RNAs when measured across the genome (data not shown). However, repetitive sources of siRNAs should produce dense clusters of small RNAs. In contrast, miRNAs are produced from cleavage at specific sites of a precursor, usually resulting in one prominent miRNA and sometimes a low abundance miRNA* from a specific region. As a consequence, comparing the absence or total abundance of individual small RNA sequences across libraries is less informative for siRNAs than it is for miRNAs. In order to compare siRNA abundances, we developed a proximity-based algorithm to build clusters of small RNAs, with the goal of comparing across libraries the presence, absence or total abundance of small RNAs in the clusters with overlapping genomic locations (see Methods). The characteristics of these clusters may help differentiate novel miRNAs from siRNAs, as sparse clusters may characterize miRNAs and dense clusters may characterize siRNAs.

Genes matched by small RNAs contained an average of one sparse cluster (Table 1C). In contrast, many transposons contained more than one cluster, and typically these were dense clusters. In the intergenic, unannotated regions of the Arabidopsis genome, more than 4,300 clusters of small RNAs were identified in the inflorescence library alone, suggesting a previously unrecognized transcriptional activity for a large proportion of the intergenic space. We also found that a high proportion of dense clusters overlapped the 5′ end of annotated genes and transposable elements, possibly representing siRNA-silenced promoters (Table 1C). The edges of these and other dense clusters likely represent the boundary of biologically-defined silenced sequences and may help refine genomic annotations.

Our analysis may underestimate the functional impact of small RNAs because we utilized perfectly matching signatures, and it is known that small RNAs are active against imperfectly-matched targets. Table 3 (See FIG. 21) provides our mismatch analysis of small RNA MPSS signatures. We examined the effects of a one-base difference (OBD) between the signature and a genomic match for this dataset (Table 3). With these mismatches, many small RNAs match in a highly degenerate manner. Of the thousands of signatures from sparse clusters of small RNAs matching genes or IGRs, more than two-thirds of the OBD matches were to other genes or IGRs containing few small RNAs. This pattern of OBD matches was consistent with that observed for signatures derived from known miRNAs. In contrast, the majority of the most repetitive signatures from dense clusters had OBD matches to regions already contained within dense clusters of perfectly matching small RNAs. If all mismatching small RNAs are as active as those with perfect matches, the level of small RNA-based transcriptional and genomic regulation is far more extensive than already suggested by our analyses based on perfect matches. In particular, large families of repetitive elements would be silenced by such numerous siRNAs and it is unlikely that they would be active under normal developmental or environmental conditions. As observed in other species, only low copy and unusual mobile elements are likely to escape silencing and retain transcriptional activity; we determined that 289 annotated Arabidopsis transposons lack small RNAs, most of which had relatively few homologs in this genome, and only a small proportion of which had expression data in mRNA MPSS libraries.

Differential accumulation of small RNAs. We next examined the differences in the small RNA populations isolated from the inflorescence and seedling libraries. Of particular interest were small RNAs that showed differences in accumulation indicative of tissue-specific regulation. A set of small RNAs matching to approximately 17% of 4,063 genes was found in only one of the two libraries (Table 4), and of these genes, four times as many were specific to inflorescence as to seedling. Comparison of clusters across the libraries demonstrated that the proportion of sparse clusters that are tissue-specific (11%) is lower than that of genes, and only 7% of dense clusters were tissue specific (Table 4). Most of the dense clusters varied only 1- to 10-fold between libraries, suggesting that these dense clusters may not be developmentally regulated, at least in these two diverse tissues. Interestingly, the genes with the most abundant seedling-specific small RNAs were PAIL and PAI2 (At1g07780 and At5g05590) which are known to be strongly regulated by epigenetic events in other Arabidopsis ecotypes. Some repetitive sequences also demonstrated tissue-specific regulation; for example, both At1 g77095, a copia-like retrotransposon, and TR2558, the tandem repeat downstream of At4g04990, specifically matched small RNAs that were found only in the inflorescence library. It was a general pattern that the inflorescence library contained more diverse small RNAs and these small RNAs matched more genes in a tissue-specific manner than the seedling library. This could reflect a greater variety of specialized cell types in the inflorescence tissue, or an increased use of small RNAs in all cell types within the inflorescence.

TABLE 4
Differential or constant clusters or genes in two libraries.

| | | | | | Higher in | | Higher in | |
|---|---|---|---|---|---|---|---|---|
| | | Total in | | | inflorescence | | seedling | |
| | | both | | Tissue | 10X to | | 10X to | |
| Type | | libraries^{a} | Undifferentiated^{b} | specific^{c} | 100X | >100X | 100X | >100X |
| Gene - complete^{d} | | 4,063 | 3,280 | 690 | 62 | 2 | 28 | 1 |
| Clusters | sparse | 16,213 | 13,873 | 1,844 | 291 | 2 | 201 | 2 |
| | moderate | 1,778 | 1,377 | 260 | 128 | 0 | 13 | 0 |
| | dense | 2,517 | 2,197 | 180 | 121 | 17 | 2 | 0 |

| | | | | | | | |
|---|---|---|---|---|---|---|---|
| Total clusters | 20,508 | 17,447 | 2,284 | 540 | 19 | 216 | 2 |

Clusters containing signatures matching to tRNAs, rRNAs, snRNAs or snoRNAs were not considered. For each library, the number of clusters or genes was calculated by the fold difference of the sum of abundances for all signatures comparing inflorescence and seedling.
^{a}The total number of genes or clusters matched by the two libraries. This includes values in columns to the right, plus all of the genes or clusters that were specific to only one of the two libraries; fold differences could not be calculated for tissue-specific genes or clusters.
^{b}This category includes small RNAs with 1X to 10X difference between the two libraries, or <10 TPQ in both libraries.
^{c}This category includes only genes or clusters that had no small RNAs in one library and small RNAs totaling ≧10 TPQ in the other library.
^{d}The complete list of genes and abundance values used in this calculation is provided in Supplemental File 2. Signatures were grouped by genes independent of the clusters. Therefore, each column contains a unique set of gene IDs.

The small RNA MPSS data clearly represent a mixture of both miRNAs and siRNAs. One source of siRNAs may be antisense transcripts that could form dsRNA with sense transcripts. Several groups have reported an abundance of antisense transcripts in Arabidopsis. If this dsRNA is formed, it could be degraded to form siRNAs that could decrease sense RNA abundance. Alternatively, interference by RNA polymerase II transcription activity on the antisense strand could restrict sense-strand transcription. Among the genes with mRNA MPSS data, about 10% also had matching small RNA signatures in libraries made from similar developmental stages (Table 5). However, we found a similarly low proportion of genes with both antisense mRNAs and small RNAs. This suggests that antisense transcripts may regulate gene activity predominantly by transcriptional interference, rather than through the production of dsRNA and small RNAs. Consistent with this, the mRNA level of genes with antisense transcripts was approximately the same whether or not they matched to small RNAs (data not shown).

TABLE 5
Comparison of small RNA and mRNA expression data.

| | | | | | | | |
|---|---|---|---|---|---|---|---|
| | | mRNA | Small | mRNA (+) | mRNA (+) | mRNA (−) | mRNA (−) |
| Tissue | Region | (+)^{a} | (+)^{b} | Small (+) | Small (−) | Small (−) | Small (+) |

A. Using mRNA MPSS signatures with single or unique matches to the genome.

| | | | | | | | |
|---|---|---|---|---|---|---|---|
| Inflorescence | Genes | 10,597 | 4,195 | 1,119 | 9,478 | 12,162 | 3,076 |
| | Genes with antisense | 1,937 | — | 228 | 1,709 | — | 3,967 |
| | IGRs | 186 | 2,865 | 33 | 153 | 20,417 | 2,832 |
| Seedling | Genes | 7,647 | 2,283 | 563 | 7,084 | 16,468 | 1,720 |
| | Genes with antisense | 3,073 | — | 265 | 2,808 | — | 2,018 |
| | IGRs | 133 | 1,630 | 16 | 117 | 21,688 | 1,614 |

B. Using all mRNA MPSS signatures.

| Inflorescence | Genes | 12,535 | 4,195 | 1,428 | 11,107 | 10,533 | 2,767 |
|---|---|---|---|---|---|---|---|
| | Genes with antisense | 2,542 | — | 327 | 2,215 | — | 3,868 |
| | IGRs | 490 | 2,865 | 131 | 359 | 20,211 | 2,734 |
| Seedling | Genes | 8,715 | 2,283 | 724 | 7,991 | 15,561 | 1,559 |
| | Genes with antisense | 3,603 | — | 314 | 3,289 | — | 1,969 |
| | IGRs | 300 | 1,630 | 49 | 251 | 21,554 | 1,581 |
| Values were calculated using the 25,835 genes and pseudogenes (removing genes classified as t/sn/sno/rRNAs, retrotransposons and transposons) and 23,435 IGRs in the TIGR version 5.0 annotation. For small RNA data, signatures were clustered by gene ID and intergenic region. | | | | | | | |
| ^{a}The “+” for mRNA MPSS indicates the presence of a signature uniquely matching to a gene and expressed at levels considered “significant” and “reliable” (Meyers et al., 2004, Gen. Research 14: 1641). This publication also describes the classification system used for mRNA MPSS signatures (Class 1 to 7), which indicate whether the signatures match in an intron, exon or intergenic region and specify the strand that is matched. For genes with antisense | | | | | | | |
| # expression, we used the sum of the Class 1/2/5/7 signatures for sense strand expression, Class 3/6 for antisense expression, and for IGRs, the presence of a Class 4 signature. | | | | | | | |

^{b}Small RNA presence in genes was based on the presence of any number of signatures at any abundance level, and included matches within the gene or UTRs. Signatures from both strands were summed. Because many pseudogenes are expressed, this set was included with genes in this analysis, and therefore the total numbers for genes in this table are higher than those of Supplemental Table 3A, which considers genes and pseudogenes separately.

We combined several computational and experimental approaches to separate siRNAs from miRNAs. Initially we compared our data with a previous study that predicted miRNAs by filtering whole genome data for sequences that form hairpin-like secondary structures, exhibited conservation with rice, and had other characteristics (AtSet1, AtSet2, and AtSet3 to AtSet6, respectively, described in ref. 48). Most of the matches between our experimental data and their predictions were found with only folding and conservation as filters, and their additional filters removed relatively few small RNAs (Table 6). The results of this comparison were consistent with Arabidopsis miRNAs numbering in the hundreds, but this approach was rudimentary.

TABLE 6
Experimental and computational data comparisons
identify potential miRNAs.

| | | | Both | |
|---|---|---|---|---|
| | | Both sets, exact | sets, ±4 bp | Both sets, any |
| Dataset^{a} | Tissue^{b} | match^{c} | match^{c} | overlap^{c} |
| AtSet1 | Inflorescence | 444/479 | 686/2,554 | 1,506/6,945 |
| (389,648) | Seedling | 178/214 | 253/1,009 | 551/2,697 |
| | Both | 791/892 | 1,158/4,593 | 2,406/12,289 |
| AtSet2 | Inflorescence | 37/43 | 64/121 | 99/152 |
| (3,851) | Seedling | 22/36 | 32/52 | 44/74 |
| | Both | 107/140 | 166/538 | 216/698 |
| AtSet3 | Inflorescence | 37/42 | 63/118 | 95/144 |
| (2,588) | Seedling | 22/36 | 32/50 | 43/68 |
| | Both | 106/138 | 164/524 | 210/672 |
| AtSet4 | Inflorescence | 36/41 | 58/110 | 82/128 |
| (2,506) | Seedling | 22/36 | 32/49 | 41/64 |
| | Both | 105/137 | 159/514 | 195/650 |
| AtSet5 | Inflorescence | 17/32 | 41/78 | 45/72 |
| (1,145) | Seedling | 16/27 | 23/35 | 27/37 |
| | Both | 85/109 | 123/417 | 132/504 |
| AtSet6 | Inflorescence | 13/15 | 24/15 | 24/12 |
| (278) | Seedling | 10/11 | 17/5 | 21/2 |
| | Both | 61/69 | 90/208 | 94/222 |
| ^{a}Numbers under each AtSet# indicate the number of sequences in each dataset defined by Jones-Rhoades and Bartel (2004, Mol. Cell 14: 787). Each set is a subset of the previous group of sequences. Briefly, AtSet1 sequences folded into hairpins, AtSet2 is conserved in rice, and the additional AtSet#s indicate miRNA-specific filters as described (Jones-Rhoades and Bartel, 2004). | | | | |
| ^{b}Tissue indicates signatures that were found in only one of the two libraries or were found in both libraries. | | | | |
| ^{c}Indicates the number of Arabidopsis sequences that were overlapping in both the small RNA MPSS data (17-base signatures) and the Jones-Rhoades and Bartel (2004) computational predictions (20-base sequences). The first number in each cell indicates the number of distinct small RNA signatures that matched, while the second number indicates the number of distinct AtSet# 20-mers that were matched. “Exact match” indicates the | | | | |
| # 5′ end was identical for both sequences; the comparison in the “±4 bp match” allowed up to four nucleotides of difference in the 5′ end; “any overlap” indicated the 20-mer and small RNA signature had at least one nucleotide of overlap, based on the location of the genomic match. | | | | |

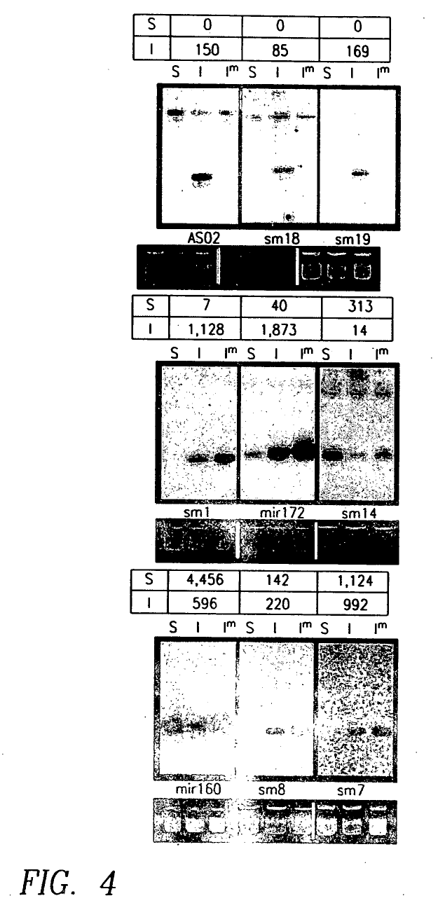

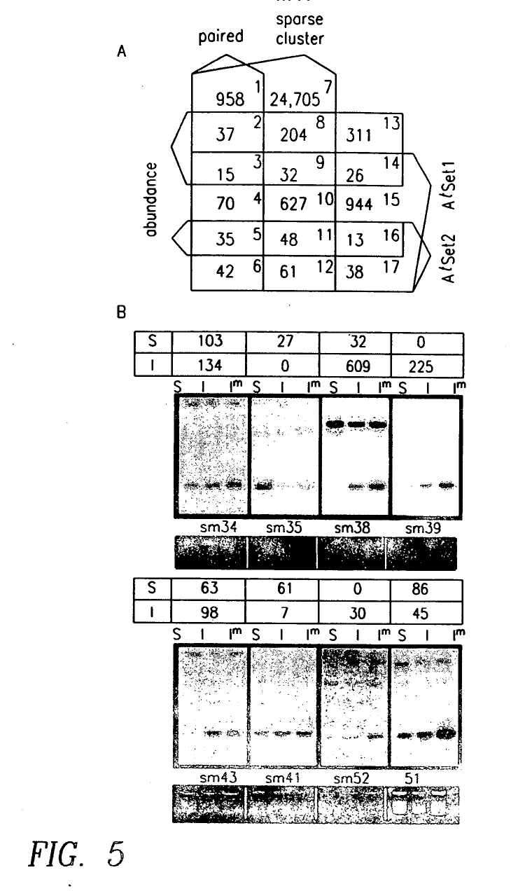

We developed a less exclusionary approach to enrich for miRNAs present in the small RNA MPSS data based on an overlapping set of filters. This method allowed us to implement and use multiple data filters in parallel and showed the numbers of small RNAs passing a subset of the filters (FIG. 5A). FIG. 5 is a five-way Venn diagram of selection criteria for small RNAs. A) The number of distinct signatures matching the criteria is indicated in each cell; small numbers in upper right corners are used in B for additional descriptions. The figure excludes 39,622 distinct signatures that did not pass any of the criteria (i.e. the majority of those in moderate or dense clusters). “Paired” indicates that two small RNAs or “sets” of small RNAs were located within 20-180 nt on the same strand, with a difference in abundance of 1:10 or greater; a “set” was defined as the consensus sequence of two or more overlapping signatures with 5′ ends within two nucleotides of each other. “Sparse cluster” is defined in the text. “Abundance” indicates the normalized abundance level in one of the libraries was equal or greater than 25 TPQ. Small RNA signatures in the AtSet1 and AtSet2 groups were present in one of the two libraries and when mapped in the genome, overlapped by at least one nucleotide with the mapped 20-nt sequences defined in Jones-Rhoades and Bartel (39). B) RNA gel blots were used to confirm new miRNA candidates identified using filters in the Venn diagram in part A. Small RNAs in the top row of blots were from box 3 of the Venn diagram. In the bottom row of blots, small RNAs #43 and #41 were from box 2, and small RNAs #52 and #51 were from box 9. Other designations are as indicated in FIG. 4. The ethidium bromide stained gels of the 5S/tRNA are indicated below each blot. The five-way Venn diagram in FIG. 5A shows that most known miRNAs were located in sparse clusters and a large percentage of the known miRNAs were captured by our abundance filter. The “paired” filter designed to identify small RNAs near another small RNA that could be a miRNA* identified many additional known miRNAs (Table 7). The contents of box #3, retained by all three filters, and box #9 retained by the sparse and abundance filters, represent good candidates for novel miRNAs and representatives of both were examined by RNA gel blots in FIG. 5B and folding predictions in FIG. 4. FIG. 4 sets forth differential miRNA and siRNA blots. RNA gel blots of low molecular weight RNA isolated from inflorescence tissues (I) and 2-week-old seedlings (S) were probed with labeled oligonucleotides. The blots also included RNA from inflorescence tissues of the rdr2 mutant (Im). The normalized abundance level from the MPSS data for each small RNA is listed above the blots and ethidium bromide staining of the 5S/tRNA region of the gels is shown below.

TABLE 7
Small RNAs in groups defined by five-way filters.

| | | | | Known | | 10-100 | Signatures | Signatures |
|---|---|---|---|---|---|---|---|---|
| | Distinct | Present in | Known | miRNA | 1-10 fold | fold | only in | only in |
| Group | signatures | AtSet6^{a} | miRNAs^{b} | families^{b} | difference | difference | inflorescence | seedling |
| 1 | 958 | — | 0 | 0 | 69 | 7 | 749 | 133 |
| 2 | 37 | — | 0 | 0 | 12 | 1 | 7 | 17 |
| 3 | 15 | — | 2 | 2 | 8 | 2 | 2 | 3 |
| 4 | 70 | — | 2 | 2 | 9 | 3 | 42 | 16 |
| 5 | 35 | 26 | 23 | 15 | 24 | 6 | 4 | 1 |
| 6 | 42 | 14 | 14 | 11 | 7 | 0 | 20 | 15 |
| 7 | 24,705 | — | 1 | 1 | 513 | 34 | 18,515 | 5,643 |
| 8 | 204 | — | 1 | 1 | 47 | 18 | 34 | 105 |
| 9 | 32 | — | 2 | 2 | 12 | 5 | 7 | 8 |
| 10 | 627 | — | 1 | 1 | 34 | 5 | 444 | 144 |
| 11 | 48 | 37 | 28 | 16 | 32 | 3 | 9 | 4 |
| 12 | 61 | 25 | 29 | 14 | 7 | 0 | 39 | 15 |
| 13 | 311 | — | 0 | 0 | 113 | 43 | 29 | 126 |
| 14 | 26 | — | 0 | 0 | 13 | 5 | 2 | 6 |
| 15 | 944 | — | 0 | 0 | 75 | 6 | 626 | 237 |
| 16 | 13 | 11 | 5 | 2 | 11 | 2 | 0 | 0 |
| 17 | 38 | 8 | 6 | 3 | 2 | 1 | 28 | 7 |

The small RNAs and groups are as described in FIG. 5A.
^{a}AtSet6 is a set of candidate miRNAs defined by Jones and Bartel (2004, Mol. Cell 14: 787).
^{b}Includes all perfect matches of small RNA signatures to miRNAs including matches with annotated 5′ ends. Some signatures match to multiple genomic locations, so the same known miRNAs may be matched by multiple groups; therefore, the total number of known miRNAs and miRNA families is less than the sum of these columns.

The large number of small RNA sequences obtained by MPSS identified more than 10-fold more small RNAs than previously described. However, this data did not reveal if we had achieved saturation of the small RNAs. Therefore, we carried out a second sequencing run on the seedling library that yielded 802,978 signatures matching to 20,379 genomic locations. Of these, 7,549 genomic matches were not identified in the first run (Table 8B) and they corresponded to 838 genes and 3,287 clusters not previously identified. Therefore, our analysis was not saturating and numerous Arabidopsis small RNAs remain to be identified. In maize and other large genomes, small RNAs are likely to be even more diverse due to the generation of diverse siRNAs from repetitive sequences that comprise the bulk of the genome. This may require even deeper sequencing of small RNAs in order to achieve saturation, although the siRNAs matching to the large families of repetitive sequences may be less interesting than small RNAs matching genes.

TABLE 8
Summary statistics for small RNA MPSS libraries.

| | | | | |
|---|---|---|---|---|
| | | Signatures | Distinct | Genome |
| # | Library | Sequenced^{a} | Signatures^{b} | Matches^{c} |

A. Inflorescence and seedling signatures.

| | | | | |
|---|---|---|---|---|
| 1 | Inflorescence | 721,044 | 67,528 | 56,920 |
| 2 | Seedling | 686,124 | 27,833 | 17,101 |

| | | | |
|---|---|---|---|
| Total of #1 and #2 | 1,407,168 | 91,445 | 70,633 |

B. Additional signatures from a second sequencing run from seedlings.

| | | | | |
|---|---|---|---|---|
| 3 | Seedling | 802,978 | 33,640 | 20,379 |
| 4 | Combined | 1,489,102 | 42,062 | 24,650 |
| | Seedling | | | |

| | | | |
|---|---|---|---|
| Total of all libraries | 2,210,146 | 104,800 | 77,434 |

^{a}The signatures sequenced for each library reflects the sum of two sequencing reactions.
^{b}“Distinct” refers to the number of different sequences found within the set. “Total” refers to the union of the different libraries.
^{c}Distinct signatures that perfectly match to at least one location in the genome, and includes signatures matching to tRNAs, rRNAs, snRNAs or snoRNAs.

Our data indicate that the small RNA component of the genome and its regulatory role is more extensive and complex than previously demonstrated. For example, many regions of the genome considered inactive or featureless were found in our analyses to be sites of considerable small RNA activity. In plants or any other organism that utilizes small RNAs as an endogenous regulatory mechanism, it should be possible to develop a more complete picture of gene and small RNA regulation by combining small RNA MPSS data from diverse samples with the genomic sequence and mRNA transcript data. For example, the small RNA MPSS data can add a new level of analysis to studies of molecular systems biology. Additional experiments, such as the analysis of small RNAs metabolism mutants, should lead to a better understanding of the sources, biological activities, turnover rates, and signaling pathways for the full range of small RNAs that we have described.

### EXAMPLE 2

Sequencing of Arabidopsis rdr2 mutants by MPSS and 454. Previous reports have indicated that rdr2 mutants show a dramatic reduction in endogenous siRNAs and a corresponding increase in miRNAs, Xie, Z., et al. 2004, Genetic and Functional Diversification of Small RNA Pathways in Plants. PLoS Biol 2: E104. It was reasoned that deep sequencing in this mutant would reveal the full complement of miRNAs in Arabidopsis. Two methods were utilized for the high-throughout sequencing of small RNAs, Meyers, B., et al., 2006, Sweating the Small Stuff: microRNA Discovery in Plants. Curr Opin Biotechnol 17: 139-146, including Massively Parallel Signature Sequencing Lu, C., et al., 2005, Elucidation of the Small RNA Component of the Transcriptome. Science 309: 1567-1569, and the 454 technology, Margulies, M. et al., 2005, Genome Sequencing in Microfabricated High-Density Picolitre Reactors. Nature 437: 376-380.

MPSS provides extraordinary depth, sequencing a half million or more molecules per library, while 454 has longer reads and thereby provides information about length. Both methods provide quantitative data based on the frequency of the molecules that were sequenced. The small RNA molecules were isolated by size fractionation, sequentially ligated to RNA adapters at the 5′ and 3′ ends, and used to make cDNA template for sequencing. Libraries were generated using mixed stage inflorescences, which are known to be a rich source of small RNAs Lu, C., et al., 2005, Elucidation of the Small RNA Component of the Transcriptome. Science 309: 1567-1569. MPSS produced 915,856 17-nucleotide signatures from rdr2 (Table 9), which is comparable to the 721,044 signatures previously obtained for wildtype Arabidopsis inflorescence. However, the rdr2 complexity was reduced by more than 80% compared to wildtype in terms of sequence diversity (9,066 different genome-matched sequences in rdr2 compared to 56,920 in wildtype). This dramatic difference was despite the larger total number of sequencing reads.

TABLE 9
Summary statistics of MPSS and 454 libraries of rdr2
and wildtype inflorescence.

| | | | | |
|---|---|---|---|---|
| | | Signatures | Distinct | Genome |
| # | Library | Sequenced^{a} | Signatures^{b} | Matches^{c} |

A. MPSS libraries.

| | | | | |
|---|---|---|---|---|
| 1 | Wildtype (FLR) | 721,044 | 67,528 | 56,920 |
| 2 | rdr2 | 915,856 | 15,325 | 9,066 |

| | | | |
|---|---|---|---|
| Total of #1 and #2 | 1,636,900 | 80,741 | 64,274 |

B. 454 libraries.

| | | | | |
|---|---|---|---|---|
| 3 | Wildtype(Col-0) | 11,631 | 9,323 | 5,713 |
| 4 | rdr2 | 7,134 | 2,003 | 686 |

| | | | |
|---|---|---|---|
| Total of #3 and #4 | 18,765 | 11,064 | 6,253 |

^{a}The signatures sequenced for each library reflects the sum of two sequencing reactions. “Total” is the sum of the different libraries. Numbers for the 454 data indicate only those sequences for which both 5′ and 3′ adapters were identified and removed, and the insert was ≧15 bp in length.
^{b}“Distinct” refers to the number of different sequences found within the set. “Total” is the union of the libraries.
^{c}Distinct signatures are counted that perfectly match to at least one location in the genome, and includes signatures matching to tRNAs, rRNAs, snRNAs or snoRNAs. “Total” is the union of the libraries.

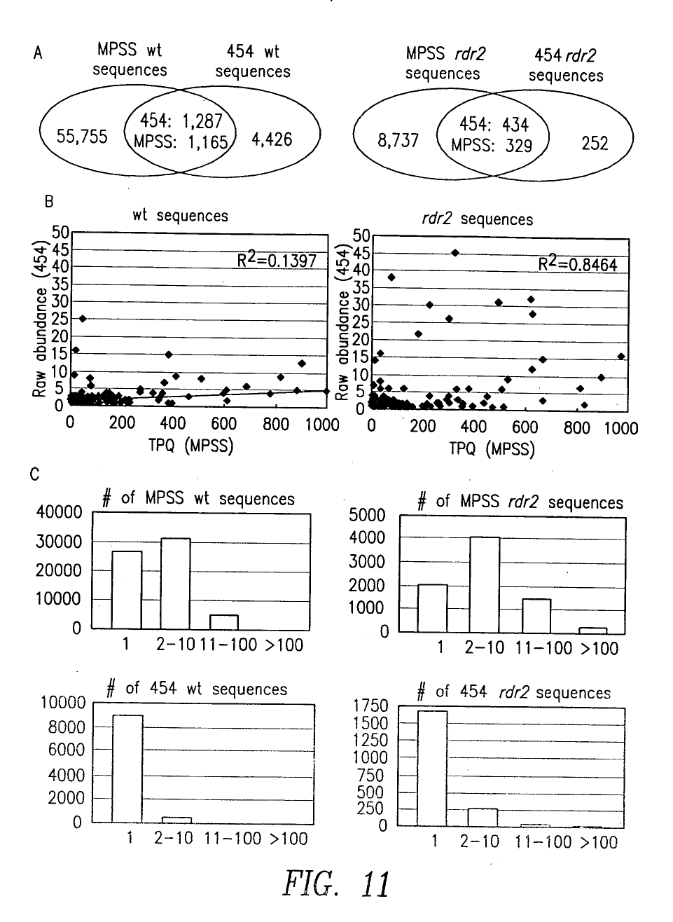

Similarly, the 454 sequencing data demonstrated a reduced complexity for rdr2 small RNAs. Using 454, 11,631 small RNAs from wildtype inflorescence were sequenced (5,713 distinct, genome-matching) and 7,134 from rdr2 (686 distinct, genome-matching). The rdr2 diversity was less than 13% that of wildtype, although in the case of the 454 data, fewer small RNAs were sequenced than with MPSS. The MPSS and 454 data correlated much better for the rdr2 mutant than the wildtype, probably because the reduced complexity of rdr2 allowed a more saturating level of sampling for even low levels of sequences (FIG. 11).

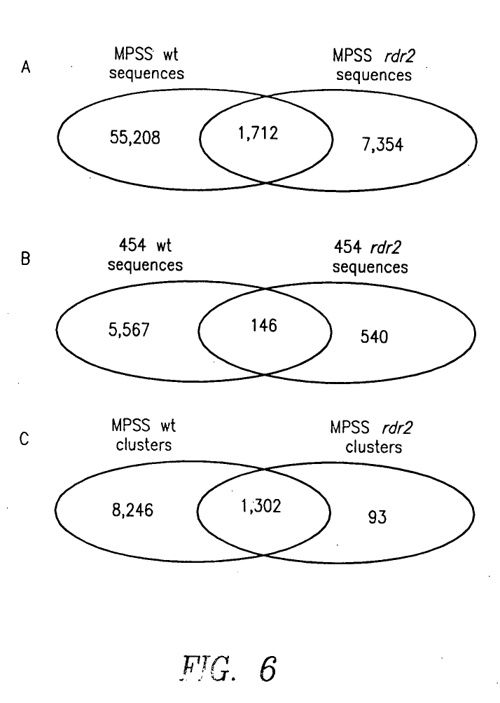

Because rdr2 is known to lack many heterochromatic siRNAs Xie, Z., et al., 2004, Genetic and Functional Diversification of Small RNA Pathways in Plants. PLoS Biol 2: E104, wildtype and rdr2 sequences were compared to determine if the small RNAs remaining in rdr2 are primarily a subset of those in wildtype. As measured by both MPSS and 454, approximately 20% of the rdr2 small RNAs were also observed in the wildtype library (FIGS. 6A and 6B). While not being bound by any particular theory, it is hypothesized that this low level of similarity was the largely the result of different siRNAs that represent the same regions. Therefore, it was determined whether the genomic loci generating small RNAs in rdr2 were the same as wildtype. To do this, we clustered the small RNAs in both rdr2 and wildtype using a proximity-based algorithm RNAs Lu, C., et al., 2005, Elucidation of the Small RNA Component of the Transcriptome. Science 309: 1567-1569 and compared clusters across the two libraries for the MPSS data. This analysis demonstrated that nearly all of the clusters (93%) containing at least three small RNAs that were detected in rdr2 were also detected in the wildtype inflorescence (FIG. 6C). Therefore, most of the small-RNA producing loci in rdr2 are also producing small RNAs in wildtype inflorescences. Most of the rdr2-only clusters were low abundance sequences that may not have been detected in wildtype due to the complexity of wildtype small RNAs and an unsaturated sample size.

Next, the population of miRNAs in the rdr2 mutant was examined and compared to wildtype. The most obvious trend was the expected enrichment of nearly all miRNAs in rdr2 compared to the wildtype (Tables 10 and 12). The overall enrichment of miRNAs in rdr2 was 1.8-fold, based on the proportion of small RNAs represented by known miRNAs (Table 11), a level similar to the 2.2-fold enrichment reported for a low level of sequencing. Eight miRNAs were enriched more than 5-fold in rdr2, including miR158, miR163, miR171, miR172, miR173, miR393, miR399, and miR402 (Table 10). The most abundant miRNA in rdr2 was miR172. This miRNA was also the most abundant in a dcl2/3/4 triple mutant Henderson, I. R., et al., 2006. Dissecting Arabidopsis DICER function in small RNA processing, gene silencing, and DNA methylation pafterning. Nat Genet In press., which, as discussed below, has a small RNA profile similar to rdr2. Both of these mutants lack many common siRNAs, and perhaps this indirectly and positively impacts miR172 abundance. At the other extreme, miR167 had a lower abundance in rdr2 than wildtype, and this was also observed in dcl2/3/4. Across the remaining miRNAs, relatively few qualitative differences were observed in terms of miRNAs that were present or absent (Tables 10 and 12). For example, the MPSS data showed that only two known miRNA families were present in rdr2 that had not been detected in wildtype inflorescence (miR157, miR400), while only miR395 was observed in wildtype but not the rdr2 454 library (and this may be due to the low sampling depth of the 454 data). Fourteen known miRNAs were never observed in either wildtype or rdr2 libraries (Table 10 and 12); this could indicate that these miRNAs are not expressed in the tissues or conditions that we sampled, some of these are not bona fide miRNAs as previously suggested, or sequence-based biases in cloning and/or sequencing steps led to their absence.

TABLE 10
miRNA families matched by small RNAs from rdr2 and wildtype inflorescence.

| | MPSS wt | MPSS rdr2 | 454 wt | 454 rdr2 | 454 dcl2/3/4 | 454 rdr6 | 454 dcl1-7 |
|---|---|---|---|---|---|---|---|
| miRNA | (TPQ) | (TPQ) | (raw) | (raw) | (raw) | (raw) | (raw) |
| miR156 | 45 | 976 | 1 | 9 | 1 | 11 | 1 |
| miR157 | 0 | 684 | 2 | 4 | 4 | 38 | 0 |
| miR158 | 74 | 3247 | 3 | 8 | 8 | 71 | 0 |
| miR159 | 61 | 82 | 246 | 281 | 452 | 398 | 41 |
| miR160 | 597 | 1389 | 4 | 5 | 16 | 11 | 2 |
| miR161 | 913 | 4248 | 22 | 54 | 73 | 208 | 36 |
| miR162 | 275 | 918 | 4 | 6 | 21 | 15 | 2 |
| miR163 | 74 | 15044 | 52 | 210 | 233 | 82 | 0 |
| miR164 | 467 | 1560 | 3 | 6 | 11 | 2 | 0 |
| miR165 | 1037 | 1059 | 10 | 25 | 55 | 38 | 5 |
| miR166 | 10620 | 3993 | 135 | 174 | 441 | 263 | 16 |
| miR167 | 59561 | 11061 | 172 | 134 | 331 | 1270 | 2 |
| miR168 | 2267 | 2091 | 8 | 2 | 37 | 17 | 36 |
| miR169 | 7650 | 14488 | 121 | 264 | 20 | 519 | 2 |
| miR170 | 15704 | 10180 | 52 | 98 | 61 | 122 | 0 |
| miR171 | 313 | 6477 | 76 | 89 | 97 | 28 | 10 |
| miR172 | 1920 | 93582 | 534 | 2100 | 1921 | 329 | 33 |
| miR173 | 518 | 4010 | 9 | 44 | 19 | 44 | 0 |
| miR319 | 372 | 433 | 8 | 25 | 16 | 8 | 0 |
| miR390 | 11349 | 17445 | 25 | 158 | 84 | 7 | 0 |
| miR393 | 49 | 972 | 4 | 8 | 10 | 31 | 0 |
| miR394 | 80 | 382 | 1 | 3 | 3 | 3 | 1 |
| miR395 | 13 | 23 | 1 | 0 | 0 | 0 | 0 |
| miR396 | 820 | 1611 | 9 | 5 | 52 | 28 | 4 |
| miR397 | 0 | 0 | 0 | 0 | 2 | 0 | 0 |
| miR398 | 111 | 228 | 1 | 2 | 35 | 18 | 0 |
| miR399 | 9 | 91 | 0 | 0 | 1 | 7 | 0 |
| miR400 | 0 | 109 | 0 | 0 | 0 | 16 | 1 |
| miR401 | 0 | 0 | 0 | 0 | 0 | 0 | 0 |
| miR402 | 6 | 123 | 0 | 0 | 0 | 0 | 0 |
| miR403 | 73 | 306 | 2 | 2 | 2 | 4 | 0 |
| miR404 | 0 | 0 | 0 | 0 | 0 | 0 | 0 |
| miR405 | 0 | 0 | 0 | 0 | 0 | 0 | 0 |
| miR406 | 0 | 0 | 0 | 0 | 0 | 0 | 0 |
| miR407 | 0 | 0 | 0 | 0 | 0 | 0 | 0 |
| miR408 | 381 | 115 | 1 | 1 | 9 | 1 | 3 |
| miR413 | 0 | 0 | 0 | 0 | 0 | 0 | 0 |
| miR414 | 0 | 0 | 0 | 0 | 0 | 0 | 0 |
| miR415 | 0 | 0 | 0 | 0 | 0 | 0 | 0 |
| miR416 | 0 | 0 | 0 | 0 | 0 | 0 | 0 |
| miR417 | 0 | 0 | 0 | 0 | 0 | 0 | 0 |
| miR418 | 0 | 0 | 0 | 0 | 0 | 0 | 0 |
| miR419 | 0 | 0 | 0 | 0 | 0 | 0 | 0 |
| miR420 | 0 | 0 | 0 | 0 | 0 | 0 | 0 |
| miR426 | 0 | 0 | 0 | 0 | 0 | 0 | 0 |
| miR447 | 0 | 0 | 0 | 0 | 0 | 0 | 0 |
| TOTAL FROM | | | 7488 | 4573 | 6214 | 6441 | 8663 |
| GENOME^{a} | | | | | | | |

“wt” indicates wildtype.
Values indicate TPQ (MPSS) or raw (454) abundance for perfect matches to known miRNAs with matches located within one nucleotide of the annotated 5′ end of the miRNA. Loci with the same name were combined for this analysis; sequences matching individual loci are described in Table S1.
^{a}Because the 454 values are raw values and not normalized, this row indicates the number of genome-matching small RNAs sequenced in each 454 library as a reference for the miRNA abundance.

TABLE 11
Small RNAs from MPSS libraries matching different types of repeats.

| | Wildtype | | rdr2 | |
|---|---|---|---|---|
| | #distinct | Sum of | #distinct | Sum of |
| Type | signatures | abundance^{c} | signatures | abundance^{c} |
| Known miRNA | 60 | 114,732 | 75 | 196,194 |
| Known ta-siRNA | 77 | 1,002 | 415 | 22,130 |
| locus | | | | |
| Gene | 11,455 | 135,340 | 3,350 | 185,367 |
| Pseudogene | 1,936 | 8,846 | 53 | 349 |
| Intergenic regions | 30,632 | 240,505 | 3,583 | 252,315 |
| Tandem repeats | 9,423 | 42,229 | 1,050 | 18,244 |
| Inverted repeats | 3,851 | 24,069 | 2,252 | 21,688 |
| Retrotransposons^{a} | 11,533 | 42,769 | 189 | 1,905 |
| Transposon^{a} | 8,737 | 33,198 | 119 | 2,943 |
| Centromeric^{b} | 5,200 | 21,615 | 80 | 431 |
| rRNA, tRNA, | 1,622 | — | 258 | — |
| snoRNA or snRNA | | | | |

^{a}Numbers of retrotransposons and transposons include sequences annotated as genes in the TIGR annotation as well as those intergenic regions identified as retrotransposons and transposons by low stringency analysis with RepeatMasker.
^{b}Centromeric repeats were defined based on regions matching the 180 bp centromeric repeats by BLAST analysis with an E-value <e⁻¹⁰.
^{c}“Sum of abundance” is the sum of TPQ-normalized abundances for all locations of all matching signatures. Signatures with multiple matches in the genome were counted for each type of genomic region in which they matched. Values are not indicated for the type “rRNA, tRNA, snoRNA or snRNA” because the abundances for these signatures were excluded from our analysis and were not normalized.

TABLE 12
Known miRNAs sequences from wildtype and rdr2.

| MPSS | | 454 | | | | |
|---|---|---|---|---|---|---|
| rdr2 | FLR | rdr2 | Col0 | rdr6 | dcl2/3/4 | dcl1-7 |

A. Perfect matches to known miRNAs.
Columns from left to right indicate the name and family member
of the known miRNA name, the normalized abundance in TPQ
in the rdr2 and wildtype inflorescence (FLR) MPSS libraries,
and the raw abundance in the 454 libraries including the rdr2
mutant, wildtype inflorescence (Col-0), rdr6, dcl2/3/4,
and dcl1-7.

| | | | | | | | |
|---|---|---|---|---|---|---|---|
| mir_id | | | | | | | |
| miR156a | 492 | 45 | 5 | 0 | 10 | 0 | 0 |
| miR156b | 492 | 45 | 6 | 0 | 10 | 0 | 0 |
| miR156c | 492 | 45 | 5 | 0 | 10 | 0 | 0 |
| miR156d | 492 | 45 | 5 | 0 | 10 | 0 | 0 |
| miR156e | 492 | 45 | 5 | 0 | 10 | 0 | 0 |
| miR156f | 492 | 45 | 5 | 0 | 10 | 0 | 0 |
| miR156g | 0 | 0 | 0 | 0 | 0 | 0 | 0 |
| miR156h | 173 | 0 | 0 | 0 | 0 | 1 | 0 |
| miR157a | 531 | 0 | 3 | 1 | 36 | 3 | 0 |
| miR157b | 531 | 0 | 3 | 1 | 36 | 3 | 0 |
| miR157c | 531 | 0 | 3 | 2 | 37 | 3 | 0 |
| miR157d | 4 | 0 | 0 | 0 | 0 | 0 | 0 |
| miR158a | 3107 | 64 | 7 | 3 | 67 | 6 | 0 |
| miR158b | 10 | 0 | 0 | 0 | 0 | 0 | 0 |
| miR159a | 0 | 0 | 233 | 205 | 322 | 377 | 38 |
| miR159b | 0 | 0 | 61 | 48 | 112 | 103 | 13 |
| miR159c | 0 | 0 | 3 | 4 | 38 | 11 | 10 |
| miR160a | 1373 | 596 | 5 | 4 | 10 | 15 | 1 |
| miR160b | 1373 | 596 | 5 | 4 | 10 | 15 | 1 |
| miR160c | 1373 | 596 | 5 | 4 | 10 | 15 | 1 |
| miR161 | 2695 | 16 | 31 | 9 | 133 | 38 | 22 |
| miR162a | 893 | 271 | 5 | 4 | 15 | 20 | 2 |
| miR162b | 893 | 271 | 5 | 4 | 15 | 20 | 2 |
| miR163 | 14955 | 45 | 209 | 52 | 82 | 232 | 0 |
| miR164a | 1560 | 465 | 6 | 3 | 2 | 11 | 0 |
| miR164b | 1560 | 465 | 6 | 3 | 2 | 8 | 0 |
| miR164c | 1560 | 465 | 1 | 1 | 0 | 0 | 0 |
| miR165a | 326 | 410 | 25 | 9 | 38 | 53 | 5 |
| miR165b | 326 | 410 | 20 | 8 | 27 | 53 | 5 |
| miR166a | 2083 | 8546 | 156 | 123 | 258 | 408 | 14 |
| miR166b | 2083 | 8546 | 156 | 123 | 258 | 408 | 14 |
| miR166c | 2083 | 8546 | 156 | 123 | 258 | 408 | 14 |
| miR166d | 2083 | 8546 | 156 | 123 | 258 | 408 | 14 |
| miR166e | 2083 | 8546 | 153 | 126 | 219 | 407 | 12 |
| miR166f | 2083 | 8546 | 153 | 126 | 219 | 407 | 12 |
| miR166g | 2083 | 8546 | 153 | 126 | 219 | 407 | 12 |
| miR167a | 11039 | 59392 | 127 | 156 | 1235 | 331 | 2 |
| miR167b | 11039 | 59392 | 116 | 161 | 1238 | 253 | 2 |
| miR167c | 12 | 16 | 0 | 0 | 0 | 0 | 0 |
| miR167d | 11039 | 59392 | 10 | 10 | 175 | 14 | 0 |
| miR168a | 2025 | 2205 | 2 | 8 | 17 | 37 | 36 |
| miR168b | 2025 | 2205 | 2 | 8 | 17 | 37 | 36 |
| miR169a | 10905 | 4485 | 41 | 18 | 249 | 9 | 0 |
| miR169b | 10905 | 4485 | 4 | 2 | 21 | 1 | 0 |
| miR169c | 10905 | 4485 | 4 | 2 | 23 | 1 | 0 |
| miR169d | 621 | 611 | 8 | 2 | 2 | 4 | 1 |
| miR169e | 621 | 611 | 8 | 2 | 2 | 4 | 1 |
| miR169f | 621 | 611 | 8 | 2 | 2 | 4 | 1 |
| miR169g | 621 | 611 | 8 | 2 | 2 | 4 | 2 |
| miR169h | 2653 | 2091 | 176 | 77 | 193 | 6 | 0 |
| miR169i | 2653 | 2091 | 196 | 86 | 237 | 6 | 0 |
| miR169j | 2653 | 2091 | 197 | 88 | 237 | 6 | 0 |
| miR169k | 2653 | 2091 | 176 | 77 | 193 | 6 | 0 |
| miR169l | 2653 | 2091 | 197 | 88 | 237 | 6 | 0 |
| miR169m | 2653 | 2091 | 177 | 86 | 203 | 6 | 0 |
| miR169n | 2653 | 2091 | 197 | 88 | 237 | 6 | 0 |
| miR170 | 10180 | 15603 | 96 | 51 | 119 | 61 | 0 |
| miR171a | 3220 | 30 | 66 | 73 | 19 | 72 | 2 |
| miR171b | 3257 | 84 | 23 | 3 | 7 | 23 | 8 |
| miR171c | 3257 | 84 | 23 | 3 | 7 | 23 | 8 |
| miR172a | 92371 | 1873 | 1712 | 410 | 324 | 1516 | 10 |
| miR172b | 92371 | 1873 | 1712 | 410 | 324 | 1516 | 10 |
| miR172c | 92371 | 1873 | 548 | 118 | 7 | 502 | 22 |
| miR172d | 92371 | 1873 | 548 | 118 | 7 | 502 | 22 |
| miR172e | 1101 | 47 | 42 | 25 | 3 | 77 | 1 |
| miR173 | 4009 | 509 | 40 | 8 | 43 | 17 | 0 |
| miR319a | 301 | 367 | 8 | 1 | 2 | 7 | 0 |
| miR319b | 301 | 367 | 8 | 1 | 2 | 7 | 0 |
| miR319c | 301 | 367 | 10 | 6 | 3 | 4 | 0 |
| miR390a | 16637 | 11038 | 141 | 23 | 6 | 67 | 0 |
| miR390b | 16637 | 11038 | 141 | 23 | 6 | 67 | 0 |
| miR393a | 972 | 45 | 8 | 4 | 30 | 10 | 0 |
| miR393b | 972 | 45 | 8 | 4 | 30 | 10 | 0 |
| miR394a | 333 | 69 | 3 | 1 | 3 | 3 | 1 |
| miR394b | 333 | 69 | 3 | 1 | 3 | 3 | 1 |
| miR395a | 2 | 0 | 0 | 1 | 0 | 0 | 0 |
| miR395b | 21 | 13 | 0 | 0 | 0 | 0 | 0 |
| miR395c | 21 | 13 | 0 | 0 | 0 | 0 | 0 |
| miR395d | 2 | 0 | 0 | 1 | 0 | 0 | 0 |
| miR395e | 2 | 0 | 0 | 1 | 0 | 0 | 0 |
| miR395f | 21 | 13 | 0 | 0 | 0 | 0 | 0 |
| miR396a | 1611 | 819 | 1 | 1 | 7 | 6 | 3 |
| miR396b | 1611 | 819 | 4 | 9 | 20 | 46 | 1 |
| miR397a | 0 | 0 | 0 | 0 | 0 | 1 | 0 |
| miR397b | 0 | 0 | 0 | 0 | 0 | 1 | 0 |
| miR398a | 228 | 111 | 0 | 0 | 1 | 1 | 0 |
| miR398b | 228 | 111 | 2 | 1 | 18 | 35 | 0 |
| miR398c | 228 | 111 | 2 | 1 | 18 | 35 | 0 |
| miR399a | 3 | 0 | 0 | 0 | 1 | 0 | 0 |
| miR399b | 87 | 9 | 0 | 0 | 3 | 1 | 0 |
| miR399c | 87 | 9 | 0 | 0 | 3 | 1 | 0 |
| miR399d | 3 | 0 | 0 | 0 | 0 | 0 | 0 |
| miR399e | 3 | 0 | 0 | 0 | 0 | 0 | 0 |
| miR399f | 3 | 0 | 0 | 0 | 2 | 0 | 0 |
| miR400 | 109 | 0 | 0 | 0 | 16 | 0 | 1 |
| miR401 | 0 | 0 | 0 | 0 | 0 | 0 | 0 |
| miR402 | 117 | 6 | 0 | 0 | 0 | 0 | 0 |
| miR403 | 306 | 73 | 2 | 2 | 4 | 2 | 0 |
| miR404 | 0 | 0 | 0 | 0 | 0 | 0 | 0 |
| miR405a | 0 | 0 | 0 | 0 | 0 | 0 | 0 |
| miR405b | 0 | 0 | 0 | 0 | 0 | 0 | 0 |
| miR405d | 0 | 0 | 0 | 0 | 0 | 0 | 0 |
| miR406 | 0 | 0 | 0 | 0 | 0 | 0 | 0 |
| miR407 | 0 | 0 | 0 | 0 | 0 | 0 | 0 |
| miR408 | 12 | 0 | 1 | 0 | 1 | 6 | 3 |
| miR413 | 0 | 0 | 0 | 0 | 0 | 0 | 0 |
| miR414 | 0 | 0 | 0 | 0 | 0 | 0 | 0 |
| miR415 | 0 | 0 | 0 | 0 | 0 | 0 | 0 |
| miR416 | 0 | 0 | 0 | 0 | 0 | 0 | 0 |
| miR417 | 0 | 0 | 0 | 0 | 0 | 0 | 0 |
| miR418 | 0 | 0 | 0 | 0 | 0 | 0 | 0 |
| miR419 | 0 | 0 | 0 | 0 | 0 | 0 | 0 |
| miR420 | 0 | 0 | 0 | 0 | 0 | 0 | 0 |
| miR426 | 0 | 0 | 0 | 0 | 0 | 0 | 0 |
| miR447a | 0 | 0 | 0 | 0 | 0 | 0 | 0 |
| miR447b | 0 | 0 | 0 | 0 | 0 | 0 | 0 |
| miR447c | 0 | 0 | 0 | 0 | 0 | 0 | 0 |

B. Known miRNAs sequences from wildtype and rdr2
allowing for small differences in start sites.
This is a version of the table above in part (A), but allowing
small RNAs that match in up to the +2 to −2 positions
compared to the annotated miRNA.

| | | | | | | | |
|---|---|---|---|---|---|---|---|
| miRNA | | | | | | | |
| miR156a | 496 | 45 | 5 | 0 | 10 | 0 | 0 |
| miR156b | 496 | 45 | 6 | 0 | 10 | 0 | 0 |
| miR156c | 496 | 45 | 5 | 0 | 10 | 0 | 0 |
| miR156d | 787 | 45 | 8 | 0 | 11 | 0 | 1 |
| miR156e | 493 | 45 | 5 | 0 | 10 | 0 | 0 |
| miR156f | 493 | 45 | 5 | 0 | 10 | 0 | 0 |
| miR156g | 1 | 0 | 0 | 0 | 0 | 0 | 0 |
| miR156h | 187 | 0 | 0 | 1 | 0 | 1 | 0 |
| miR157a | 535 | 0 | 3 | 1 | 36 | 3 | 0 |
| miR157b | 535 | 0 | 3 | 1 | 36 | 3 | 0 |
| miR157c | 535 | 0 | 3 | 2 | 37 | 3 | 0 |
| miR157d | 153 | 0 | 1 | 0 | 1 | 1 | 0 |
| miR158a | 3241 | 64 | 8 | 3 | 73 | 8 | 0 |
| miR158b | 10 | 10 | 0 | 0 | 4 | 0 | 0 |
| miR159a | 105 | 64 | 237 | 207 | 324 | 382 | 38 |
| miR159b | 105 | 64 | 61 | 48 | 114 | 103 | 13 |
| miR159c | 54 | 11 | 3 | 4 | 38 | 11 | 10 |
| miR160a | 1387 | 597 | 5 | 4 | 11 | 16 | 2 |
| miR160b | 1375 | 596 | 5 | 4 | 10 | 15 | 1 |
| miR160c | 1387 | 597 | 5 | 4 | 11 | 16 | 2 |
| miR161 | 4248 | 913 | 54 | 22 | 212 | 73 | 37 |
| miR162a | 932 | 275 | 6 | 4 | 15 | 21 | 2 |
| miR162b | 932 | 275 | 6 | 4 | 15 | 21 | 2 |
| miR163 | 15092 | 83 | 210 | 52 | 82 | 234 | 0 |
| miR164a | 1560 | 467 | 6 | 3 | 2 | 11 | 0 |
| miR164b | 1560 | 467 | 6 | 3 | 2 | 8 | 0 |
| miR164c | 1560 | 467 | 1 | 1 | 0 | 0 | 0 |
| miR165a | 395 | 642 | 25 | 10 | 38 | 55 | 5 |
| miR165b | 997 | 854 | 20 | 8 | 27 | 53 | 5 |
| miR166a | 3270 | 10059 | 168 | 131 | 263 | 432 | 14 |
| miR166b | 2762 | 9214 | 164 | 124 | 260 | 416 | 15 |
| miR166c | 2159 | 9005 | 159 | 123 | 259 | 408 | 14 |
| miR166d | 2159 | 9005 | 159 | 123 | 259 | 408 | 14 |
| miR166e | 2762 | 9214 | 161 | 127 | 221 | 415 | 13 |
| miR166f | 2760 | 9166 | 161 | 127 | 221 | 415 | 13 |
| miR166g | 2159 | 9005 | 156 | 126 | 220 | 407 | 12 |
| miR167a | 11039 | 59519 | 129 | 157 | 1244 | 331 | 2 |
| miR167b | 11039 | 59519 | 118 | 162 | 1249 | 253 | 2 |
| miR167c | 12 | 45 | 0 | 0 | 1 | 0 | 0 |
| miR167d | 11049 | 59574 | 10 | 10 | 179 | 14 | 0 |
| miR168a | 2100 | 2267 | 2 | 8 | 17 | 37 | 36 |
| miR168b | 2100 | 2267 | 2 | 8 | 17 | 37 | 36 |
| miR169a | 11243 | 4892 | 47 | 19 | 256 | 9 | 0 |
| miR169b | 11243 | 4892 | 6 | 2 | 22 | 1 | 0 |
| miR169c | 11140 | 4842 | 4 | 2 | 24 | 1 | 0 |
| miR169d | 921 | 1063 | 13 | 3 | 5 | 4 | 1 |
| miR169e | 921 | 1063 | 13 | 3 | 5 | 4 | 1 |
| miR169f | 921 | 1063 | 10 | 3 | 3 | 4 | 1 |
| miR169g | 919 | 1063 | 10 | 3 | 3 | 4 | 2 |
| miR169h | 2931 | 2455 | 181 | 77 | 195 | 6 | 0 |
| miR169i | 2931 | 2455 | 202 | 86 | 240 | 6 | 0 |
| miR169j | 2895 | 2455 | 201 | 88 | 240 | 6 | 0 |
| miR169k | 2931 | 2455 | 181 | 77 | 195 | 6 | 0 |
| miR169l | 2888 | 2448 | 201 | 88 | 240 | 6 | 0 |
| miR169m | 2931 | 2455 | 182 | 86 | 205 | 6 | 0 |
| miR169n | 2895 | 2455 | 201 | 88 | 240 | 6 | 0 |
| miR170 | 10180 | 15704 | 98 | 52 | 122 | 61 | 0 |
| miR171a | 3220 | 288 | 66 | 73 | 21 | 74 | 2 |
| miR171b | 3257 | 88 | 23 | 3 | 7 | 23 | 8 |
| miR171c | 3257 | 88 | 23 | 3 | 7 | 23 | 8 |
| miR172a | 92487 | 1894 | 1732 | 413 | 326 | 1537 | 10 |
| miR172b | 92487 | 1894 | 1732 | 413 | 326 | 1537 | 10 |
| miR172c | 92487 | 1894 | 551 | 118 | 7 | 504 | 22 |
| miR172d | 92487 | 1894 | 551 | 118 | 7 | 504 | 22 |
| miR172e | 1178 | 68 | 54 | 28 | 5 | 95 | 1 |
| miR173 | 4010 | 519 | 44 | 9 | 44 | 19 | 0 |
| miR319a | 395 | 532 | 11 | 2 | 2 | 12 | 0 |
| miR319b | 301 | 372 | 10 | 2 | 2 | 7 | 0 |
| miR319c | 427 | 372 | 16 | 6 | 6 | 9 | 0 |
| miR390a | 17445 | 11349 | 158 | 25 | 7 | 84 | 0 |
| miR390b | 17445 | 11349 | 158 | 25 | 7 | 84 | 0 |
| miR393a | 972 | 49 | 8 | 4 | 31 | 10 | 0 |
| miR393b | 972 | 49 | 8 | 4 | 31 | 10 | 0 |
| miR394a | 396 | 80 | 3 | 1 | 3 | 3 | 1 |
| miR394b | 396 | 80 | 3 | 1 | 3 | 3 | 1 |
| miR395a | 2 | 0 | 0 | 1 | 0 | 0 | 0 |
| miR395b | 21 | 13 | 0 | 0 | 0 | 0 | 0 |
| miR395c | 21 | 13 | 0 | 0 | 0 | 0 | 0 |
| miR395d | 2 | 0 | 0 | 1 | 0 | 0 | 0 |
| miR395e | 2 | 0 | 0 | 1 | 0 | 0 | 0 |
| miR395f | 21 | 13 | 0 | 0 | 0 | 0 | 0 |
| miR396a | 1611 | 820 | 1 | 1 | 8 | 6 | 3 |
| miR396b | 1611 | 819 | 4 | 9 | 20 | 46 | 1 |
| miR397a | 0 | 0 | 0 | 0 | 0 | 1 | 0 |
| miR397b | 0 | 0 | 0 | 0 | 0 | 1 | 0 |
| miR398a | 228 | 111 | 0 | 0 | 1 | 1 | 0 |
| miR398b | 228 | 111 | 2 | 1 | 18 | 35 | 0 |
| miR398c | 228 | 111 | 2 | 1 | 18 | 35 | 0 |
| miR399a | 4 | 0 | 0 | 0 | 1 | 0 | 0 |
| miR399b | 87 | 9 | 0 | 0 | 3 | 1 | 0 |
| miR399c | 87 | 9 | 0 | 0 | 3 | 1 | 0 |
| miR399d | 4 | 0 | 0 | 0 | 0 | 0 | 0 |
| miR399e | 4 | 0 | 0 | 0 | 0 | 0 | 0 |
| miR399f | 4 | 0 | 0 | 0 | 3 | 0 | 0 |
| miR400 | 109 | 0 | 0 | 0 | 16 | 0 | 1 |
| miR401 | 0 | 0 | 0 | 0 | 0 | 0 | 0 |
| miR402 | 123 | 6 | 0 | 0 | 0 | 0 | 0 |
| miR403 | 307 | 73 | 2 | 2 | 4 | 2 | 0 |
| miR404 | 0 | 0 | 0 | 0 | 0 | 0 | 0 |
| miR405a | 0 | 0 | 0 | 0 | 0 | 0 | 0 |
| miR405b | 0 | 0 | 0 | 0 | 0 | 0 | 0 |
| miR405d | 0 | 0 | 0 | 0 | 0 | 0 | 0 |
| miR406 | 0 | 0 | 0 | 0 | 0 | 0 | 0 |
| miR407 | 0 | 0 | 0 | 0 | 0 | 0 | 0 |
| miR408 | 115 | 385 | 1 | 1 | 1 | 9 | 3 |
| miR413 | 0 | 0 | 0 | 0 | 0 | 0 | 0 |
| miR414 | 0 | 0 | 0 | 0 | 0 | 0 | 0 |
| miR415 | 0 | 0 | 0 | 0 | 0 | 0 | 0 |
| miR416 | 0 | 0 | 0 | 0 | 0 | 0 | 0 |
| miR417 | 0 | 0 | 0 | 0 | 0 | 0 | 0 |
| miR418 | 0 | 0 | 0 | 0 | 0 | 0 | 0 |
| miR419 | 0 | 0 | 0 | 0 | 0 | 0 | 0 |
| miR420 | 0 | 0 | 0 | 0 | 0 | 0 | 0 |
| miR426 | 0 | 0 | 0 | 0 | 0 | 0 | 0 |
| miR447a | 0 | 0 | 0 | 0 | 0 | 0 | 0 |
| miR447b | 0 | 0 | 0 | 0 | 0 | 0 | 0 |
| miR447c | 0 | 0 | 0 | 0 | 0 | 0 | 0 |

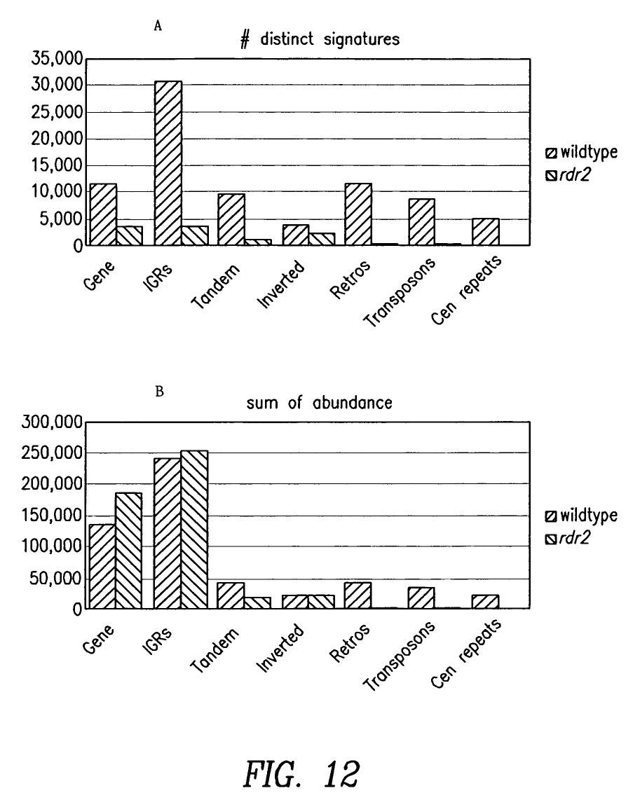

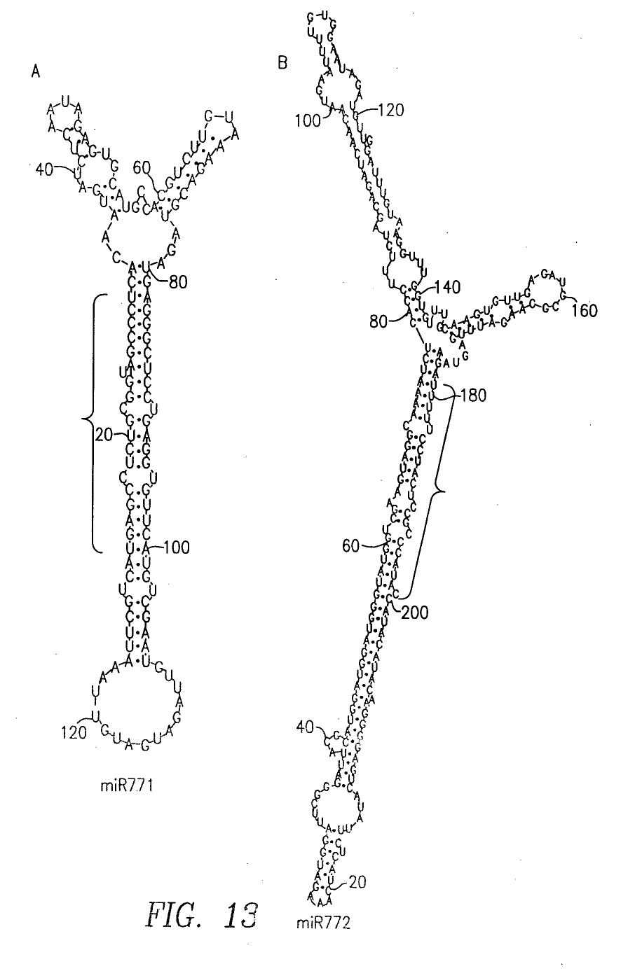

The rdr2 small RNAs showed a much more limited distribution on the Arabidopsis chromosomes compared to wildtype, due to their reduced complexity. The small RNAs from the rdr2 mutant did not show a pericentromeric concentration, which is a noticeable contrast with wildtype small RNAs; this is consistent with a loss of heterochromatic siRNAs in rdr2. However, there were many more loci matching small RNAs in rdr2 than are represented by the 117 known miRNA loci. This could indicate that many miRNAs, ta-siRNAs or other RDR2-independent small RNAs have yet to be described. As a first step to determine the nature of these RDR2-independent small RNAs, the relationship between rdr2 small RNAs and different genomic regions was examined. Compared to wildtype, small RNAs were reduced in rdr2 in each class of genomic sequence that we investigated (Table 11 and FIG. 12). Based on the normalized abundances, there was a proportionally greater reduction in small RNAs associated with pseudogenes, transposons and retrotransposons, compared to genes and unclassified intergenic regions, consistent with a loss of heterochromatic siRNAs (Table 11, FIG. 12). Small RNAs in the intergenic regions potentially represent unannotated miRNAs, or siRNAs from unannotated repeats such as tandem or inverted genomic repeats. Inverted repeats showed one of the lowest reductions in small RNAs in rdr2, while small RNAs from tandem repeats were fewer but still well-represented.

### EXAMPLE 3

Experimental Validation of Novel miRNAs.

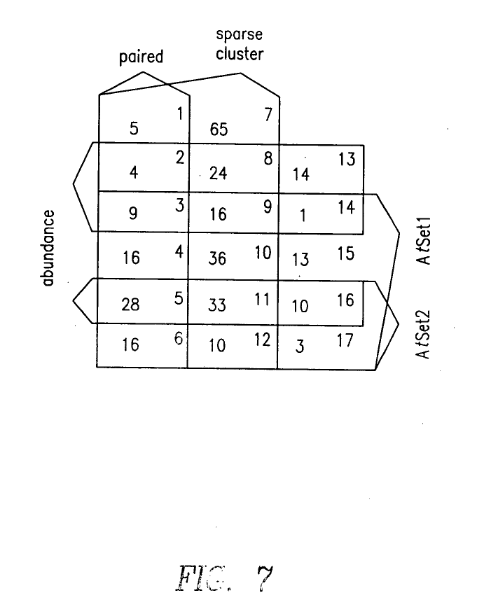

As a first step towards the identification of novel miRNAs, rdr2 MPSS sequences were compared with previously-identified wildtype small RNAs in a five-way Venn diagram (FIG. 7). Among those small RNAs that are present in both libraries, the sequences were chosen for further analysis from boxes 3-6 and 9-12; these sequences matched genomic regions that can form hairpin structures and they passed the sparse cluster filter typical of miRNAs. Eliminating known miRNA genes (101 sequences) and transposons (eight sequences) resulted in a set of 54 small RNA sequences and a total of 31 candidate genomic loci. Because most of the novel candidate miRNAs were sequenced by MPSS multiple times and all were independently detected in two different samples (rdr2 and wildtype), they represent good candidates for novel Arabidopsis miRNAs that are expressed at low levels, may not be conserved between plant species, and have not been described as miRNAs by previous approaches or experiments.

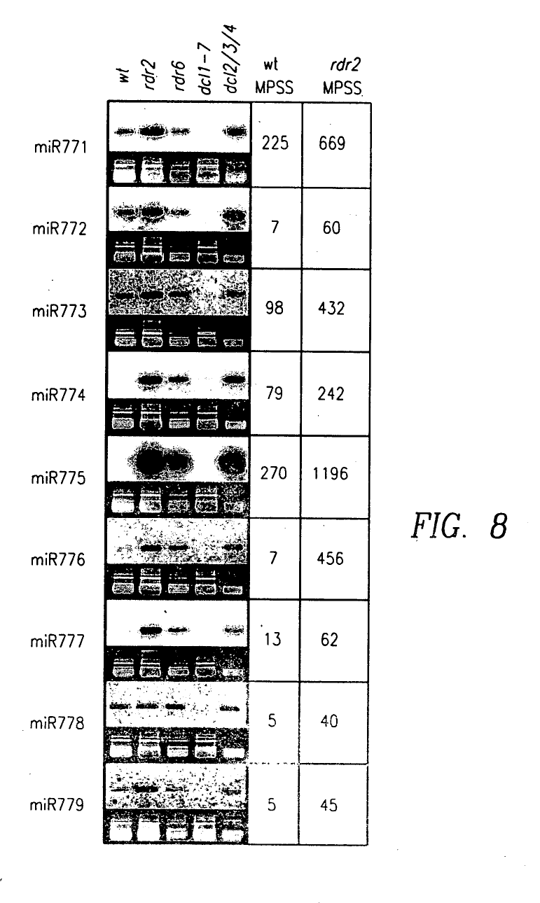

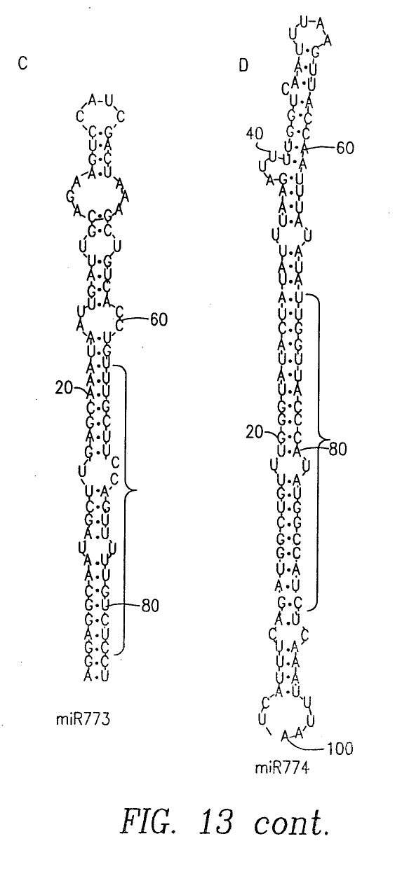

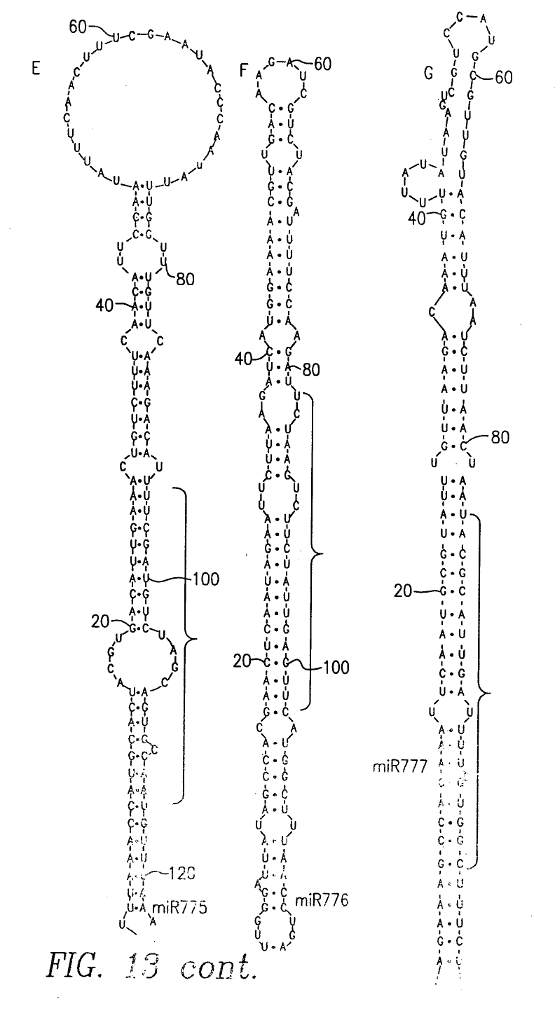

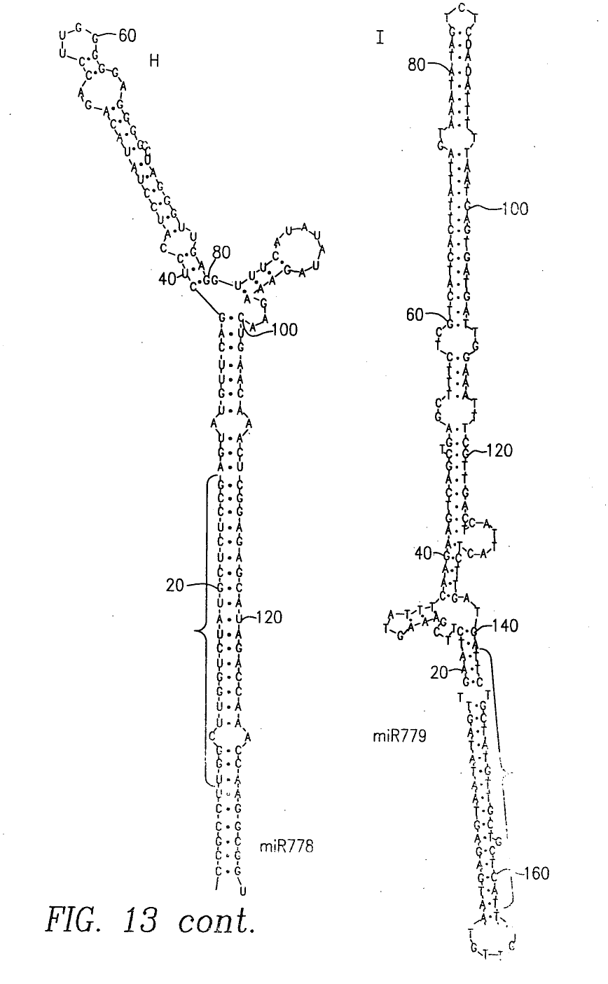

As a complementary experimental approach to validate candidate miRNAs, the expression of candidate miRNAs in different genetic backgrounds was evaluated by RNA gel blot analysis of low molecular weight RNA isolated from inflorescence tissues. Canonical miRNAs generally require DCL1 (not DCL2, 3 or 4), but not RDR2 or RDR6, while 21 nt siRNAs from ta-siRNA loci require DCL1, DCL4 and RDR6 but not RDR2. Arabidopsis mutants with defects in Dicer and RdRp genes, therefore, are important tools to distinguish among different classes of small RNAs. Of the 31 candidate hairpin-forming genomic loci from the Venn diagram, we conducted RNA gel blot analysis of 13 from boxes containing small RNA signatures with an MPSS abundance of ≧40 transcripts per quarter-million (TPQ), including three small RNAs that we previously predicted to be miRNAs. Bands within the size range of 21 to 24 nt expected for mature miRNAs were observed for 12 of 13 candidates that we tested, and of these, nine small RNAs had genetic requirements similar to those of typical, known miRNAs (FIG. 8; Table 13A); our blots indicated the small RNAs are present in inflorescence tissue of wildtype, rdr2, rdr6, and a dcl2/3/4 triple mutant, but are absent in dcl1-7. Furthermore, these nine small RNAs can form stable fold-back structures with the flanking genomic sequence, which is typical of a miRNA precursor, and contain the sequenced small RNA within one arm of the hairpin (FIG. 13). Like the majority of known miRNAs, the first 5′ nucleotide of these new miRNAs was predominantly a uracil residue. Based on the mutant analysis and folding, these are new miRNAs. We focused on boxes 3, 9, and 10 (FIG. 7) to identify new miRNAs because sequences in these boxes lacked a match in AtSet2 indicating that the Arabidopsis hairpin sequences were not well conserved with rice. Thus, it is not surprising that among these nine new Arabidopsis miRNAs, five do not have identifiable homologs in rice or Medicago truncatula based on sequence rather than hairpin comparisons. Like other non-conserved miRNAs, such as miR161 and miR163, these five miRNAs are represented by single loci rather than multigene families.

TABLE 13
New miRNAs and other rdr2-independent small RNAs identified by deep sequencing.

| | Wildtype | rdr2 | | | | | | Venn |
|---|---|---|---|---|---|---|---|---|
| | MPSS | MPSS | RNA gel blot results | | | | | position |
| Sequence | (TPQ) | (TPQ) | wt | rdr2 | rdr6 | dcl1-7 | dcl2/3/4 | in FIG. 3 |

A. New miRNAs.

| | | | | | | | | | |
|---|---|---|---|---|---|---|---|---|---|
| miRNA | | | | | | | | | |
| miR771a | TGAGCCTCTGTGGTAGCCCTC | 225 | 669 | + | + | + | − | + | 3 |
| miR772a | TTTTTCCTACTCCGCCCATAC | 7 | 60 | + | + | + | − | + | 9 |
| miR773a | TTTGCTTCCAGCTTTTGTCTC | 98 | 432 | + | + | + | − | + | 9 |
| miR774 | TTGGTTACCCATATGGCCATC | 79 | 242 | + | + | + | − | + | 9 |
| miR775 | TTCGATGTCTAGCAGTGCCAA | 270 | 1196 | − | + | + | − | + | 9 |
| miR776 | TCTAAGTCTTCTATTGATGTT | 7 | 456 | +^{a} | + | + | − | + | 10 |
| miR777 | TACGCATTGAGTTTCGTTGCT | 13 | 62 | + | + | + | − | + | 10 |
| miR778 | TGGCTTGGTTTATGTACACCG | 5 | 40 | + | + | + | − | + | 10 |
| miR779 | TTCTGCTATGTTGCTGCTCAT | 5 | 45 | + | + | + | − | + | 10 |

B. Other RDR2-independent small RNAs.

| | | | | | | | | | |
|---|---|---|---|---|---|---|---|---|---|
| small | | | | | | | | | |
| ID | | | | | | | | | |
| small49 | AGGACCATTGCGGTTGTGCAA | 57 | 343 | + | + | − | − | − | 9 |
| small57 | TGCGGGAAGCATTTGCACATG | 23 | 227 | + | + | +^{b} | + | − | 9 |
| small58 | TACCGCAAGATCAAAGTTCAC | 0 | 17 | +^{b} | + | − | − | − | 10 |
| small62 | CAACTCCAGGATTGGACCAGT | 0 | 47 | − | − | − | − | − | 10 |

See FIG. 8 for RNA gel blot analyses of these sequences.
^{a}Indicates that this small RNA was previously reported as a potential miRNA (Lu et al., 2005), but was not previously
confirmed or submitted to the miRNA registry.
^{b}Indicates the bands for these small RNAs in the indicated background were weak.

C: New miRNAs

| | | Wildtype | rdr2 | | | | | |
|---|---|---|---|---|---|---|---|---|
| | | MPSS | MPSS | RNA gel blot results | | | | |
| miRNA | Sequence | (TPQ) | (TPQ) | wt | rdr2 | rdr6 | dcl1-7 | dcl2/3/4 |
| miR780 | TTTCTTCGTGAATATCTGGCA | 5 | 134 | + | + | + | − | + |
| miR781 | TTAGAGTTTTCTGGATACTTA | 0 | 77 | +^{a} | + | + | − | + |
| miR782 | ACAAACACCTTGGATGTTCTT | 6 | 16 | + | + | + | − | + |
| miR783 | AAGCTTTGCTCGTTCATGTTC | 0 | 35 | + | + | + | − | + |

^{a}Indicates the band for these small RNAs in the indicated background were weak.

D: Predicted targets of the new miRNAs

| | | | # of | Target |
|---|---|---|---|---|
| Small RNA | Target Family^{a} | Target Gene IDs (score) | Targets | Site |
| miR780 | | None | 1 | ORF |
| miR781 | n.a. | At1g26960 (2), At5g23480 (2.5), At1g44900 (2.5) | 3 | ORF |
| miR782 | n.a. | At5g33405 (2.5) | 1 | ORF |
| miR783 | Extra-large G-protein-related | At4g01090 (2) | 1 | ORF |

^{a}“n.a.” indicates “not applicable” because the targets were hypothetical proteins or too diverse to predominantly
represent a single family

Below is a listing of the above sequences including SEQ ID NOs:

| miRNA | Sequence | SEQ ID NO. |
|---|---|---|
| miR771 | TGAGCCTCTGTGGTAGCCCTC | SEQ ID NO: 185,397 |
| miR772 | TTTTTCCTACTCCGCCCATAC | SEQ ID NO: 185,398 |
| miR773 | TTTGCTTCCAGCTTTTGTCTC | SEQ ID NO: 185,399 |
| miR774 | TTGGTTACCCATATGGCCATC | SEQ ID NO: 185,400 |
| miR775 | TTCGATGTCTAGCAGTGCCAA | SEQ ID NO: 185,401 |
| miR776 | TCTAAGTCTTCTATTGATGTT | SEQ ID NO: 185,402 |
| miR777 | TACGCATTGAGTTTCGTTGCT | SEQ ID NO: 185,403 |
| miR778 | TGGCTTGGTTTATGTACACGC | SEQ ID NO: 185,404 |
| miR779 | TTCTGCTATGTTGCTGCTCAT | SEQ ID NO: 185,405 |
| miR780 | TTTCTTCGTGAATATCTGGCA | SEQ ID NO: 185,406 |
| miR781 | TTAGAGTTTTCTGGATACTTA | SEQ ID NO: 185,407 |
| miR782 | ACAAACACCTTGGATGTTCTT | SEQ ID NO: 185,408 |
| miR783 | AAGCTTTGCTCGTTCATGTTC | SEQ ID NO: 185,409 |
| small49 | AGGACCATTGCGGTTGTGCAA | SEQ ID NO: 185,410 |
| small57 | TGCGGGAAGCATTTGCACATG | SEQ ID NO: 185,411 |
| small58 | TACCGCAAGATCAAAGTTCAC | SEQ ID NO: 185,412 |
| small62 | CAACTCCAGGATTGGACCAGT | SEQ ID NO: 185,413 |

Plant miRNAs function in the regulation of gene expression either by inducing cleavage of their mRNA targets or by translational repression. Therefore, to characterize the function of the new miRNAs identified, regulatory targets were predicted using an algorithm similar to the one described by Jones-Rhoades and Bartel (2004). In general, cleavage is predominant and can be experimentally assessed using a modified 5′-RACE approach to validate these mRNA targets. Targets were predicted with a penalty score of 2.5 or better for seven of the nine new miRNAs (Table 14A), using the 21 nt sequence derived from the 17 nt MPSS tag plus four adjacent nucleotides from the matching genomic location. The new Arabidopsis miRNA genes are expressed at relatively low abundances as demonstrated by the MPSS data and RNA gel blots (FIG. 8), and most of them were also absent or marginally represented in other small RNA libraries sequenced by traditional methods. Consequently, mapping of cleavage products generated from these new miRNAs may be challenging due to the low and/or differential expression of the predicted target mRNAs.

TABLE 14
Predicted targets of new miRNAs and ta-siRNAs.

| | | | | |
|---|---|---|---|---|
| | | | # of | Target |
| Small RNA | Target Family^{a} | Target Gene IDs (score) | Targets | Site |

A. Predicted targets of new miRNAs.

| | | | | |
|---|---|---|---|---|
| miR772 | NBS-LRR disease | At1g51480 (1), At5g43740 (1), At1g12290 | 12 | ORF |
| | resistance genes | (1.5), At1g12210 (1.5), At5g63020 (1.5), | | |
| | | At4g14610 (2), At4g10780 (2), At1g12220 | | |
| | | (2), At1g15890 (2), At1g12280 (2.5), | | |
| | | At5g47260 (2.5), At5g05400 (2.5), | | |
| miR773 | DNA (cytosine-5-)- | At4g14140 (2), At4g08990 (2.5) | 6 | ORF |
| | methyltransferase | | | |
| | and others | At4g05390 (2), At3g15330 (2.5), | | |
| | | At3g16230 (2.5) | | |
| | | At2g22730 (2) | | UTR ? |
| miR774 | F-box family genes | At3g19890 (1), At3g17490 (2) | 2 | ORF |
| miR775 | galactosyltransferase | At1g53290 (2) | 1 | ORF |
| | family gene | | | |
| miR776 | | At5g62310 (1.5) | 2 | ORF |
| | | At1g08760 (1.5) | | UTR ? |
| miR778 | SET domain- | At2g22740 (1.5), At2g35160 (2.5) | 2 | ORF |
| | containing genes | | | |
| miR779 | S-locus protein | At2g19130 (2.5) | 1 | UTR? |
| | kinase | | | |
| miR771 | | None | | |
| miR777 | | None | | |

Score is based on the system described by Jones-Rhoades and Bartel (2004). The number of predicted
targets is based on a cut-off score of 2.5.

B. Predicted targets of new ta-siRNAs.

| | | | | |
|---|---|---|---|---|
| Small49 | n.a. | At4g00600 (3) | 2 | ORF |
| | | At4g00610 (3) | | UTR? |
| Small58 | n.a. | At2g39980 (3) | 9 | UTR? |
| | | At5g20200 (3) | | ORF |

As above, the score is based on the system described by Jones-Rhoades and Bartel (2004), but the
number of predicted targets is based on a cut-off score of 3.
^{a}“n.a.” indicates “not applicable” because the targets were too diverse to predominantly
represent a single family.

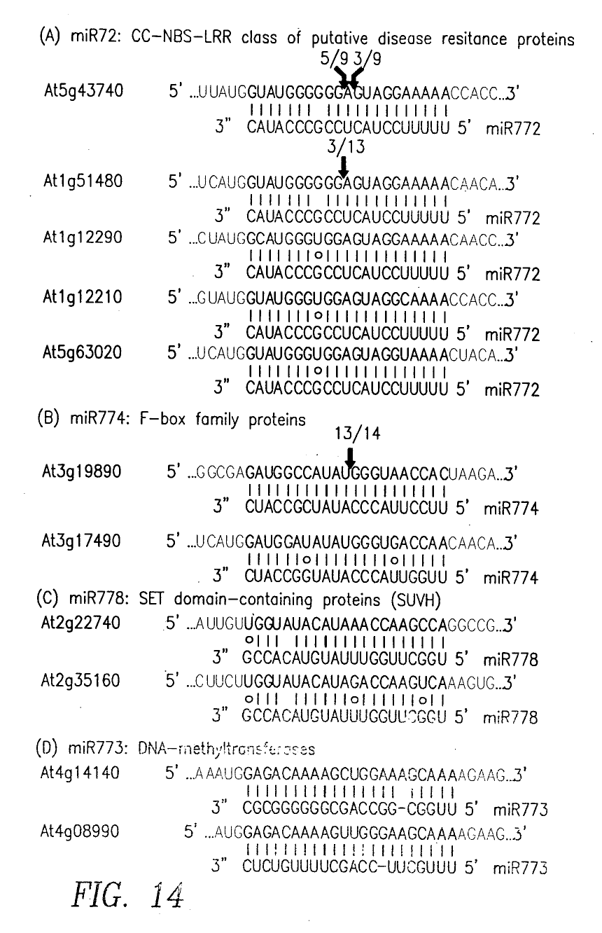

Three new miRNA targets were verified among which two have a predicted role in plant defense responses. Two transcripts encoding the CC-NBS-LRR class of putative disease resistance proteins (At5g43740 and At1 g51480) were experimentally validated as in vivo targets of miR772 (FIG. 14A). The predicted target site for miR772 (SEQ ID NO. 185,398) is the region encoding the P-loop domain which is highly conserved in this class of CC-NBS-LRR disease resistance proteins. Because of this conservation, miR772 is predicted to target at least 10 more relatives of this gene family (Table 14A); the targeting of multiple members of a gene family by a miRNA has previously been reported for several known miRNAs. Interestingly, two additional cleavage sites in At1 g51480 were mapped, one 31 nt upstream and the other 16 nt downstream of the expected miR772 cleavage site (data not shown). This may result from the activities of other small RNAs that have not yet been identified. MiR773 (SEQ ID NO. 185,399), miR774 (SEQ ID NO. 185,400), and miR778 (SEQ ID NO. 185,404) were also predicted to target several members of a gene family; for instance, miR774 is predicted to target transcripts for two genes that encode F-box proteins (FIG. 14B; Table 14A). Notably, several other F-box mRNAs are known targets of miRNA394 and 396, and target validation assays indicated that the mRNA for another member of this extended gene family (At3g19890) is being cleaved by miR774 (FIG. 14B). Although multiple attempts failed to confirm miR778 and miR773-mediated cleavage, the cleavage products of the transcripts predicted to be targets of these miRNAs may be detected in the future, under different conditions that elevate their abundance, for example. These predicted targets include components associated with silencing: two putative SU(VAR)3-9 like histone methyltransferase (SUVH5 and SUVH6) transcripts that are potential targets of miR778 and members of the family of DNA (cytosine-5)-methyltransferases that are potentially targeted by miR773. Previous reports have described miRNA targets involved in silencing, including DCL1 and Argonaute1 (AGO1), targets of miR162 and miR168, respectively.

### EXAMPLE 4

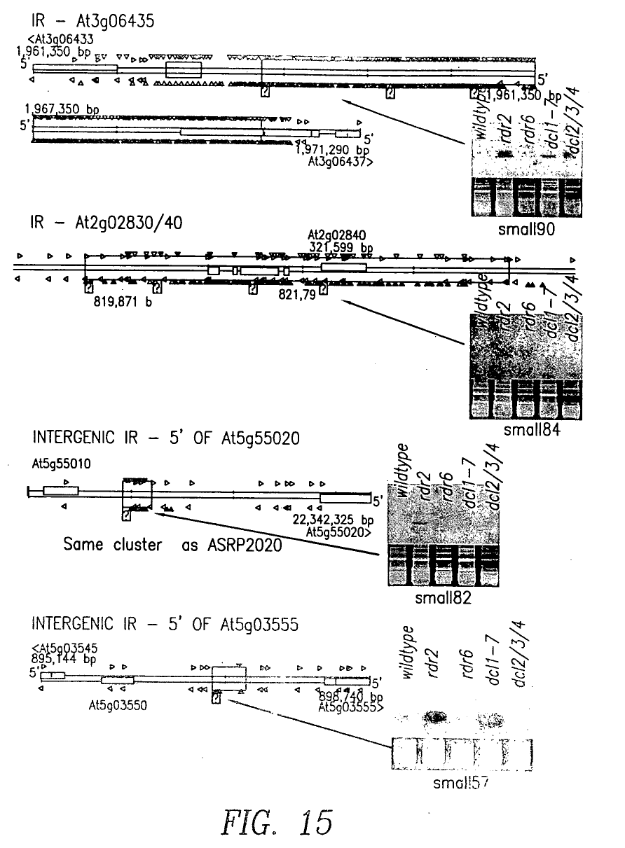

Other RDR2-independent small RNAs in Arabidopsis. A significant number of Arabidopsis endogenous siRNAs match to various kind of repeats. Xie et al. have shown the requirement of RDR2 and DCL3 for the biosynthesis of a subset of repeat-associated siRNAs. However, considering the presence of multiple RdRps in Arabidopsis and the diversity of repeats, it is unclear which populations of siRNAs generated from repeat sequences are dependent on RDR2 activity. The RDR2-dependent and RDR2-independent inverted and tandem repeats were separately characterized; these repeats are known to be sources of small RNAs. The RDR2-dependent inverted repeat set, comprising a total of 461 genomic locations, were defined as those for which: 1) the sum of abundance is ≧10 TPQ in wildtype; 2) the sum of abundance is at least 10-fold higher in wildtype than in rdr2. Similarly, a repeat was considered to be RDR2-independent only if the sum of abundance from the repeat is ≧10 TPQ and not down-regulated (rdr2/wt≧1) in rdr2. As shown in Table 15, 55 loci were found for this set (12% of the total). The repeat score of the RDR2-independent set was significantly higher than that of the RDR2-dependent set (Mann-Whitney Test: P−value=0.0048). One of the primary determinants of the score is the length of the repeat, suggesting that the RDR2-dependence of inverted repeats may be based on their length. This is consistent with a previous study suggesting that for some inverted repeats, RDR2 may contribute to the formation or stability of a complex that contains active DCL3. For genomic loci that contain long inverted duplications and can form extensive dsRNA structures (“foldbacks”), RDR2 is most likely dispensable for siRNA production (Table 15). One hypothesis is that one or more Dicers can efficiently process long dsRNA precursors even in the absence of RDR2. In agreement with this, closer examination of some RDR2-independent inverted repeats revealed that these loci usually showed complex patterns of siRNA accumulation with different size classes affected by different Dicer mutants (FIG. 15).

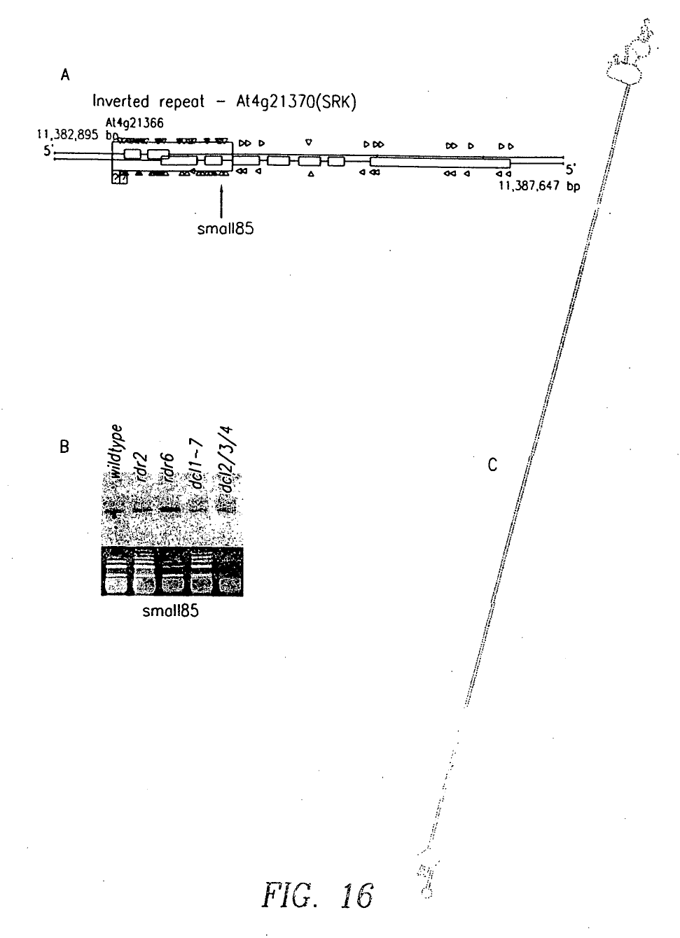

A potential foldback structure in the S-receptor kinase gene (SRK) was identified as one of the most strongly expressed RDR2-independent siRNA-producing regions (FIG. 16). The large number of sequenced small RNAs matching to this stem-loop suggests that it is a substrate for Dicer cleavage. The observation that small85, from this locus, is still evident in the dcl1-7 and dcl2/3/4 mutants but not in a quadruple dcl1/2/3/4 mutant (data not shown) suggests the involvement of multiple Dicers (FIG. 16). Functional copies of SRK and a gene called SCR are important for self-incompatibility in Brassica and Arabidopsis species (such as A. lyrata). Loss of this self-incompatibility system in Arabidopsis thaliana is one of the key factors that led to the selection of A. thaliana as a model system for plants. Suggested explanations for this loss include the fragmented SCR gene or the alternatively spliced SRK transcripts that contain premature nonsense codons that are present in A. thaliana. These data suggest that the SRK gene may be silenced by an inverted-repeat, and these small RNAs may have played a previously-unknown role in the loss of SRK function in A. thaliana.

Unlike inverted repeats from which dsRNA is readily generated simply by folding of a single RNA, tandem repeats should require an RdRp to form dsRNA structures. Indeed, tandem repeats show a higher overall dependence on RDR2 than inverted repeats (Table 15). Our RDR2-dependent tandem repeat set contained 3491 genomic locations whereas the RDR2-independent tandem repeat set contained only 82 loci (2% of the total). Interestingly, the average length of the tandem repeat unit in RDR2-dependent set is significantly larger than that of the RDR2-independent set (Mann-Whitney Test: P−value=0.0001). Therefore, high quality and long tandem repeats generally appear to require RDR2 to generate dsRNAs and sustain siRNA production. Other RdRps probably facilitate dsRNA production from these short tandem repeats because the Arabidopsis genome contains six RdRp homologs. Without being limited by any particular theory, one likely hypothesis is that different RdRps could function redundantly on tandem repeats.

TABLE 15
RDR2-dependent and RDR2-independent repeats from MPSS libraries.
A. Inverted repeats.

| | | % | | |
|---|---|---|---|---|
| | Score | of Similarity | Gap^{a} | Size |
| RDR2- | 799.4 ± 34.0 | 86.4 ± 0.45 | 5.7 ± 0.4 | 405 ± 17 |
| dependent | | | | |
| RDR2- | 1595.7 ± 232.7 | 86.7 ± 1.5 | 7.1 ± 1.1 | 713 ± 86 |
| independent | | | | |

B. Tandem repeats.

| | | % | | |
|---|---|---|---|---|
| | Score | of Similarity | Count^{b} | Size^{c} |
| RDR2- | 129.8 ± 8.1 | 84.1 ± 0.1 | 3.7 ± 0.06 | 101.4 ± 3.4 |
| dependent | | | | |
| RDR2- | 44.1 ± 12.0 | 81.6 ± 0.9 | 5.1 ± 0.73 | 32.8 ± 4.3 |
| independent | | | | |
| In each case, RDR2-dependent is defined as the sum of abundance is ≧10 TPQ in wild type and the sum of abundance is at least 10-fold higher in wildtype than in rdr2; RDR2-independent is defined as the sum of abundance from the repeat is ≧10 TPQ in rdr2 and the small RNAs are not down-regulated in rdr2 (rdr2/wt ≧1). Mean values for each category are indicated followed by standard error (±). The score was determined by the programs Einverted or Etandem, and represents | | | | |
| # a composite of length and identity for each set of repeats. The complete set of inverted and tandem repeat data is provided in Supplemental File 1. | | | | |

^{a}“Gap” indicates the average gap between arms of the inverted repeat (in nucleotides).
^{b}“Count” refers to the number of tandem repeats.
^{c}“Size” indicates the average length of the repeats at each locus (in nucleotides).

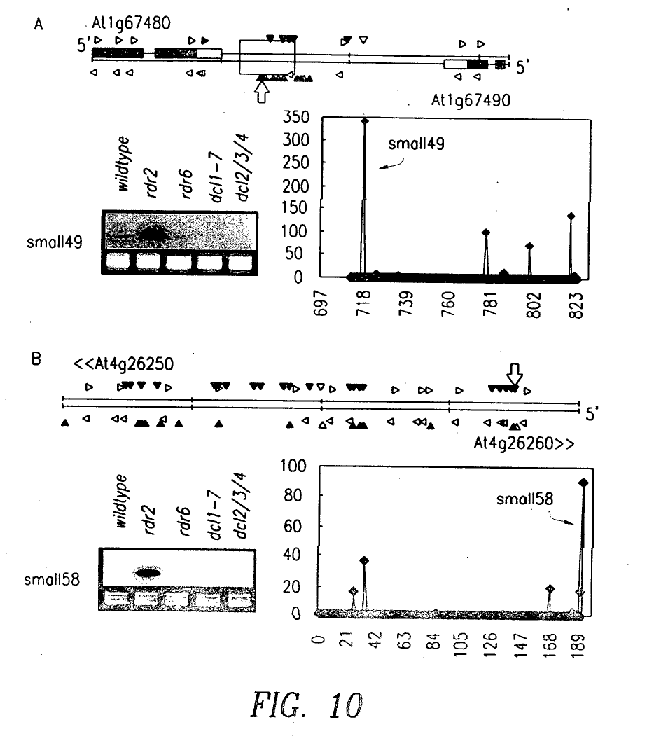

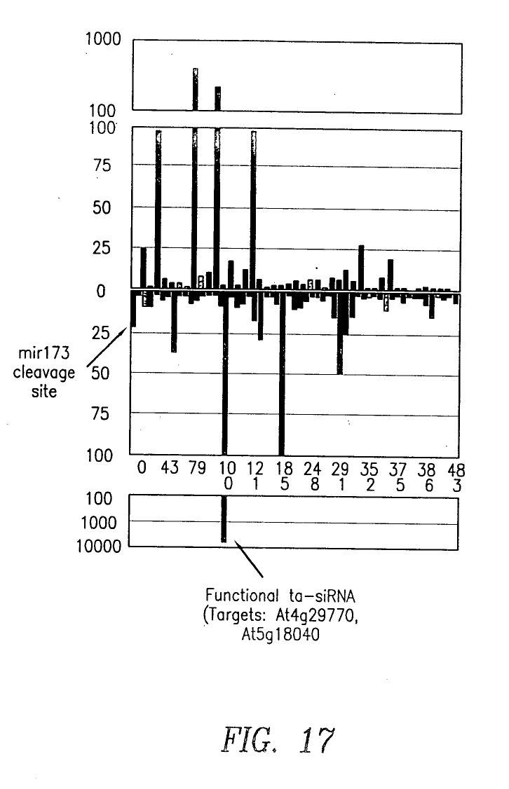

Known ta-siRNA loci were the most enriched small RNA sources in the rdr2 background. For the four previously characterized ta-siRNA loci, the sum of small RNA abundance was at least 20-fold higher in rdr2 than in wildtype based on the MPSS data (Table 16A and FIG. 17). This greatly exceeds the 1.8 fold for enrichment of total miRNA abundance mentioned earlier. Using known ta-siRNAs as reference, a set of filters to enrich for new ta-siRNAs was developed. Four filters were designed and applied to identify genomic locations representing potential ta-siRNA loci: 1) the cluster contains at least 10 distinct signatures; 2) the sum of abundance for the cluster is ≧100 TPQ; 3) the sum of abundance is at least 10-fold higher in rdr2 than in wild type; 4) the cluster does not match to known miRNAs, ta-siRNAs, transposons, retrotransposons or centromere repeats. These filters generated 28 potential ta-siRNA loci (Table 17). Interestingly, among these, 14 loci (50% of the filter output) corresponded to different members of the PPR gene family, a group of genes known to be targeted by miRNAs, ta-siRNAs and siRNAs. Seven of the 14 remaining candidate loci were further examined by RNA gel blotting. We found two candidates (small49, small58) displaying typical ta-siRNA expression patterns (present in rdr2 but very low in rdr6, dcl1 and dcl2/3/4) (Table 13B and FIG. 10). Furthermore, a clear 21 nt phased pattern was observed at the locus containing small49, consistent with Dicer activity (FIG. 10). With this low stringency filtering protocol that captures all known ta-siRNA loci, relatively few loci were found which had ta-siRNA characteristics. Therefore, we interpret these data as an indication that ta-siRNA genes are rare in the Arabidopsis genome. This result is consistent with the observation that mutations that block ta-siRNA production have a relatively weak phenotype. However, it is also possible that other ta-siRNAs were expressed at very low levels or not at all under these sampling conditions.

TABLE 16
Representation of known ta-siRNA loci in small RNA libraries.
A. MPSS libraries.

| ta- | | start | end | | Sum of | Sum of |
|---|---|---|---|---|---|---|
| siRNA | | coordinates | coordinates | # distinct | abundance | abundance |
| locus | chromosome | (bp) | (bp) | signatures^{a} | in wildtype | in rdr2 |
| TAS1a | 2 | 11728344 | 11729168 | 94 | 115 | 7633 |
| TAS1b | 1 | 18552926 | 18553725 | 63 | 217 | 13115 |
| TAS1c | 2 | 16544582 | 16545150 | 126 | 349 | 10456 |
| TAS2 | 2 | 16546598 | 16547391 | 92 | 457 | 8027 |
| TAS3 | 3 | 5862059 | 5862369 | 81 | 66 | 2094 |

B. 454 libraries.

| ta-siRNA | Wildtype | | | | |
|---|---|---|---|---|---|
| locus | (Col-0) | rdr2 | rdr6 | dcl1-7 | dcl2/3/4 |
| TAS1a | 12 | 41 | 4 | 0 | 0 |
| TAS1b | 7 | 37 | 1 | 0 | 0 |
| TAS1c | 32 | 72 | 3 | 0 | 0 |
| TAS2 | 28 | 71 | 1 | 0 | 0 |
| TAS3 | 13 | 11 | 1 | 5 | 0 |

^{a}The number of distinct signatures was calculated as the sum of distinct signatures in the wildtype and rdr2 libraries.

TABLE 17
Genomic loci with features of ta-siRNA loci.

| | | | | | | rdr2/ | |
|---|---|---|---|---|---|---|---|
| Chr. # | start | end | hits | rdr2 | wildtype | wildtype | comments^{a} |
| 1 | 4182124 | 4182323 | 11 | 147 | 7 | 21.00 | *** |
| 1 | 4354497 | 4355226 | 20 | 188 | 0 | 188.00 | PPR gene family |
| 1 | 4368786 | 4369099 | 13 | 1028 | 58 | 17.72 | |
| 1 | 5297877 | 5298129 | 65 | 302 | 22 | 13.73 | |
| 1 | 23181100 | 23182270 | 43 | 177 | 1 | 177.00 | PPR gene family |
| 1 | 23208490 | 23209751 | 55 | 563 | 51 | 11.04 | PPR gene family |
| 1 | 23279171 | 23280268 | 19 | 148 | 6 | 24.67 | PPR gene family |
| 1 | 23303291 | 23304571 | 130 | 842 | 70 | 12.03 | PPR gene family |
| 1 | 23305811 | 23307450 | 88 | 740 | 35 | 21.14 | PPR gene family |
| 1 | 23310777 | 23312267 | 105 | 437 | 34 | 12.85 | PPR gene family |
| 1 | 23389058 | 23390321 | 51 | 476 | 81 | 5.88 | PPR gene family |
| 1 | 23392690 | 23393912 | 121 | 901 | 107 | 8.42 | PPR gene family |
| 1 | 23417056 | 23418359 | 134 | 779 | 89 | 8.75 | PPR gene family |
| 1 | 23423630 | 23424830 | 79 | 859 | 50 | 17.18 | PPR gene family |
| 1 | 23493873 | 23495043 | 45 | 172 | 1 | 172.00 | PPR gene family |
| 1 | 23511578 | 23512642 | 96 | 616 | 27 | 22.81 | PPR gene family |
| 1 | 23590850 | 23591523 | 14 | 292 | 6 | 48.67 | PPR gene family |
| 1 | 25282658 | 25283382 | 30 | 713 | 105 | 6.79 | ***^{b} |
| 2 | 819173 | 823134 | 183 | 627 | 34 | 18.44 | *** |
| 2 | 7198149 | 7198613 | 61 | 282 | 13 | 21.69 | |
| 2 | 17231588 | 17231885 | 26 | 127 | 10 | 12.70 | *** |
| 4 | 1318892 | 1319151 | 27 | 133 | 8 | 16.63 | |
| 4 | 11383503 | 11384499 | 78 | 164 | 24 | 6.83 | *** |
| 4 | 13295428 | 13296124 | 16 | 230 | 14 | 16.43 | ***^{b} |
| 5 | 897027 | 897335 | 18 | 517 | 35 | 14.77 | *** |
| 5 | 15774898 | 15775413 | 50 | 282 | 21 | 13.43 | |
| 5 | 16656600 | 16658007 | 36 | 121 | 2 | 60.50 | |
| 5 | 20151669 | 20151865 | 42 | 525 | 46 | 11.41 | |

The filters used to identify these loci are as follows: 1) The sum of abundance in rdr2 ≧ 100. 2) The number of distinct small RNAs in rdr2 ≧ 10. 3) The ratio of rdr2/wt ≧ 5. 4) The loci do not correspond to miRNAs, known ta-siRNAs, transposons, retrotransposons, or centromeric repeats. “Hits” indicates the number of distinct small RNAs found at each locus in both rdr2 and wildtype.
^{a}PPR gene families are noted because they have been described as strong sources of small RNAs (Lu et al., 2005).
*** indicates that RNA gel blots were performed using a small RNA sequence selected from this locus (data not shown), which was confirmed to have the expression pattern of a canonical ts-siRNA (present in wildtype, enriched in rdr2, absent in rdr6, dcl1-7 and dcl2/3/4).
^{b}These loci also showed phasing similar to known ta-siRNAs, and are shown in more detail, along with the RNA gel blot, in FIG. 10.

### EXAMPLE 5

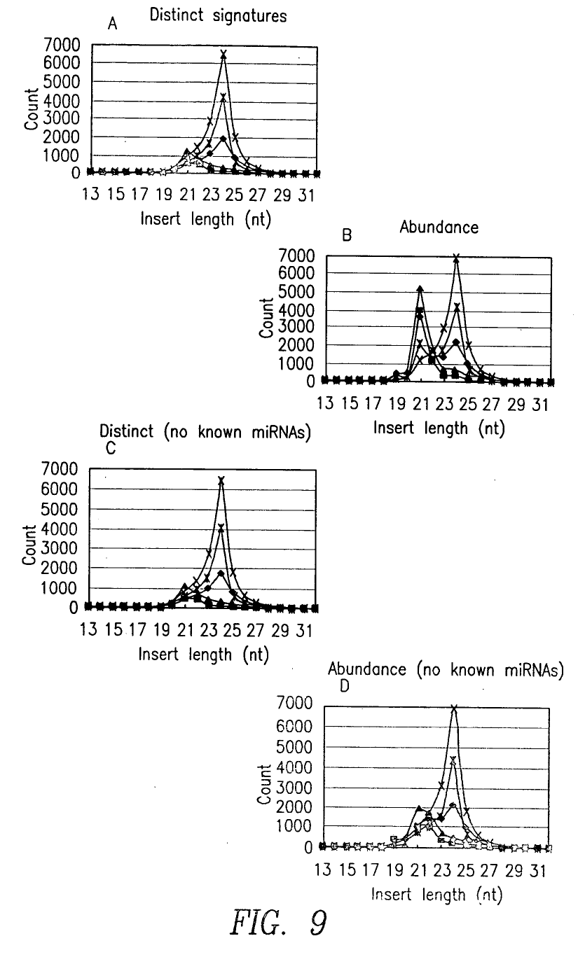

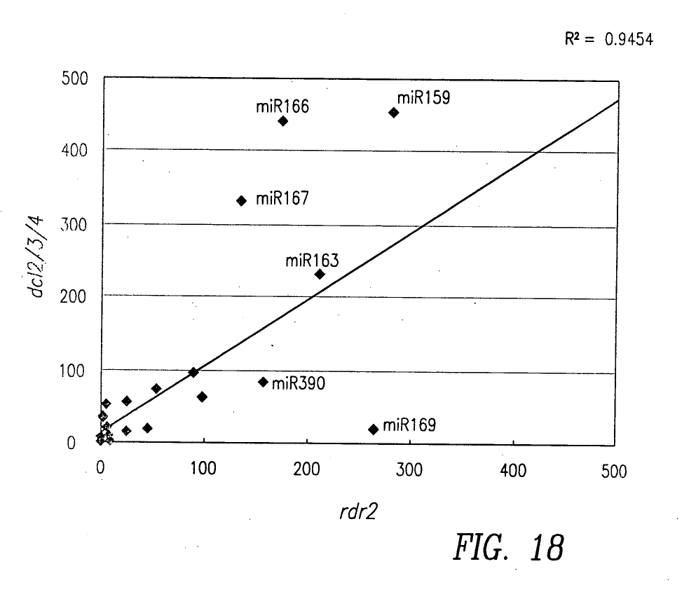

Small RNA size distribution in rdr2 and the small RNA populations in other mutants. The enrichment of miRNAs and loss of heterochromatic siRNAs in rdr2 should correlate with a shift in the sizes of the small RNA population. Canonical miRNAs are 21 nt while canonical heterochromatic siRNAs are 24 nt. Because the MPSS sequence data is limited to 17 nucleotides for small RNAs, we used the 454 sequence data to determine the size distribution of the small RNAs. As an additional comparison to wildtype and rdr2 inflorescences, small RNAs from the inflorescence of the Arabidopsis mutants rdr6 and dcl1-7 were also sequenced, and compared these to data we recently obtained for dcl2/3/4. All of these mutants are altered in important genes for small RNA biogenesis. The size distribution based on both distinct sequences and total abundances was assessed (FIG. 9). Both rdr2 and the dcl2/3/4 triple mutant showed a similar pattern of 24 nt siRNA reduction and 21 nt miRNA enrichment (FIGS. 9A and 9B). The increase in 21-mers in both mutants reflects an enrichment of miRNAs and is consistent with previous reports (Table 2, FIGS. 9A and 9B). In contrast to miRNAs, 21 nt siRNAs from known ta-siRNA loci can be readily identified from rdr2, but were absent in dcl2/3/4 (Table 16B), consistent with previous observation that DCL4 is required for ta-siRNA production. Nevertheless, a strong correlation between the 454 data of rdr2 and dcl2/3/4/was observed (R²=0.92 for all small RNAs present in both libraries; R²=0.95 for miRNAs, FIG. 18) In contrast, the dcl1-7 mutant demonstrated lower proportion of 21 nt small RNAs compared to wildtype (FIG. 9B), and most of this difference can be attributed to a substantial reduction in known miRNAs (Table 2, FIGS. 9B and 9D). This is consistent with the known reduction in the miRNA complement of dcl1-7. Both the wildtype and rdr6 mutant have substantial peaks at both 21 and 24 nt, as expected. However, analysis of ta-siRNA abundance in the rdr6 mutants has revealed that indeed very few ta-siRNAs were detected in the absence of RDR6 (Table 16B).

Even the modest depth of the 454 sequencing was sufficient to identify differential effects of specific mutants on the accumulation on miRNA families. Although DCL1 appears to be the only Dicer protein responsible for miRNA biogenesis in Arabidopsis, some miRNAs are affected less than others by the dcl1-7 mutant. The most extreme case was miR168 which did not decrease at all in dcl1-7 based on the 454 data (Table 10). These results are in agreement with Vaucheret et al., who reported no decrease in miR168 levels in three different dcl1 partial loss-of-function mutants. This fits well with the model that miR168 levels are not limited by DCL1 activity but are instead controlled by a feedback loop involving AGO1, the target of miR168; AGO1 is hypothesized to both stabilize miR168 and also slice its own mRNA using miR168 as a guide. The accumulation of miR159 and miR165/166 has also been reported to be somewhat less sensitive to dcl mutations than other miRNAs tested and we also observed these subtleties. Finally, members of the miR161 family, and miR408 are known to be rather insensitive to the dcl1-7 allele and the dcl1-9 allele respectively, results quite consistent with our 454 data. Based on the close recapitulation of published observations with this dcl1 data, it seems likely that other differential accumulation characteristics resulting from this data set represent regulatory characteristics of biological significance. These would include miR167, which is down-regulated in rdr2 compared to wild type, and miR172 which is of particularly high abundance in rdr2 and dcl2/3/4 (Table 10). Another miRNA with unusual characteristics is miR169. This miRNA is an outlier in the correlation of rdr2 and dcl2/3/4 (FIG. 18), having a very low accumulation in rdr2, with high accumulation in dcl2/3/4. Given that miR169 is also increased in rdr6 and encoded by a tandem array of genes, these accumulation results may be due to a secondary level of control by an siRNA-mediated pathway.

Prior experimental and computational efforts over the last several years have resulted in the identification of 117 miRNA genes in Arabidopsis which can be grouped into 42 families. The miRNAs SEQ ID NO: 185,397-185,409 all represent new families that presumably escaped previous discovery because of their low abundance. These new miRNAs increase the total number of Arabidopsis miRNA families by 25%. Eight of the newly described miRNAs are found only in Arabidopsis. For non-conserved miRNAs, it is more difficult to confidently predict targets because the conservation of the target site cannot be used as a filter to remove false positives. Therefore, a highly stringent score (≦2.5) was applied in target prediction. Potential regulatory targets were found for 10 of the 13 miRNAs. Some of the biological roles of the newly confirmed or predicted targets resemble those of previously identified Arabidopsis miRNAs. At least three of these are bona fide because we could map the cleavage products and we predict that others were simply beneath our threshold of detection. MiR774 (SEQ ID NO. 185,400) targets the mRNA of at least one F-box protein. Combined with six previously identified F-box genes, there are at least seven F-box mRNAs targeted by miRNAs, suggesting that the protein degradation machinery is subject to considerable miRNA regulation. Our observation that miR773 (SEQ ID NO. 185,399) mediates the cleavage of at least two, and potentially more, members of the CC-NBS-LRR class of putative disease resistance proteins suggests a previously unknown role of miRNAs in plant defense. As new and more sensitive methods for verifying miRNA targets are developed, it will be exciting to see if some of the other interesting putative targets such as the methytransferases in FIG. 14 can be verified. While, our target predictions focused on protein-coding genes, at least two miRNAs (mir173 and mir390) target precursors of ta-siRNAs; consequently, there may be additional targets for some of these new miRNAs that have not yet been identified.

RDR2-independent siRNAs. Tandem repeats are prone to epigenetic silencing mediated by RNA interference. Previous studies have shown that several siRNAs corresponding to tandem repeats in the Arabidopsis genome were absent in rdr2. It has been proposed that tandem repeats can sustain RdRp activity because the first round siRNAs can randomly initiate subsequent rounds of siRNA production and perpetuate the siRNA pool. While this model has not been proven, it is substantiated by our MPSS data indicating that almost all the tandem repeats in the Arabidopsis genome required RDR2 activity to generate siRNAs. However, for some of these tandem repeats, the small RNAs were significantly higher in rdr2 than in wildtype. Something about these tandem repeats, perhaps their relatively low quality, may allow these sequences to be silenced independently of RDR2. In this case, other components of the siRNA biogenesis machinery must be involved in the recognition and generation of siRNAs from these specific loci. This suggests that the biogenesis pathway for repeat-associated siRNAs is more complex than initially believed and the production of some repeat-associated siRNAs does not require RDR2 activity.

siRNA accumulation from inverted-repeat loci is dependent on RDR2 and DCL3. While DCL3 clearly functions as the ribonuclease to process dsRNA precursors, it is unclear why RDR2 is essential to this pathway. Another example is siRNA production from constructs used for inverted-repeat post-transcriptional gene silencing (IR-PTGS, typically used for RNAi). Although widely-used as a research tool, IR-PTGS remains one of the least understood plant RNA silencing processes. Until recently, no mutant defective in this pathway had been recovered, and IR-transgene induced siRNA accumulation is not affected by single gene mutations. Our analysis of rdr2 by MPSS may provide an explanation for these apparently contradictory observations. In agreement with previous studies, the majority of endogenous inverted-repeats, such as the siRNA02 locus, did not accumulate siRNAs in the absence of RDR2. However, we also identified a group of inverted-repeats which produced siRNAs independently of RDR2. One difference between RDR2-dependent and RDR2-independent inverted repeats is that the latter set tends to have a higher repeat score and larger size of repeat unit. Although it is difficult to rule out alternative hypotheses completely, the simplest interpretation of the data is that RDR2 and DCL3 are required for only a subset of inverted-repeats, generally with low scores and relatively short repeat units. In the case of longer and higher scoring inverted repeats, RDR2 activity (and probably DCL3) may not be required, similar to IR-transgenes. One likely scenario is that the high quality dsRNA structures generated from long inverted repeats are subject to the activity of different Dicers. Consistent with this model, recent analyses of combinatorial Dicer knockout mutants indicated that the functions of different Arabidopsis Dicer proteins are highly redundant.

The combined deep profiling data from MPSS and full-length sequencing of small RNAs from different genotypes by 454 demonstrate that small RNA sequence libraries are a rich and novel source of data that have yet to be fully exploited in Arabidopsis or any other organism. As sequencing costs drop with the advent of new short-read sequencing technologies, the approaches that we have implemented for the analysis of Arabidopsis mutants are likely to be more broadly applied for experimental investigation of different conditions, mutants, and organisms.

Methods

Plant growth. All plant material was from Arabidopsis ecotype Col-0. The rdr2, rdr6, dcl1-7, and dcl2/3/4 mutants have been described previously. Inflorescence tissue was harvested from plants grown in soil in a growth chamber with 16 hours of light for 5 weeks. Floral tissue included the inflorescence meristem and early stage floral buds (up to Stage 11/12). Total RNA was isolated using Trizol reagents (Invitrogen, Carlsbad, Calif.). Seedlings were grown at 23° C. under the same 16 hour long day conditions and were harvested after two weeks. Inflorescence and seedling material was harvested approximately at eight hours into the subjective day.

RNA gel blot analysis. Blot hybridization analysis was performed as described. Total RNA was extracted using Trizol (Invitrogen, Carlsbad, Calif.). High molecular weight (HMW) RNA was precipitated with 5% PEG8000 and 0.5M NaCl. The low molecular weight (LMW) RNA which remained in the supernatant was precipitated with ethanol. LMW RNA was resolved on 15% polyacrylamide gels, blotted to Zeta-Probe GT genomic blotting membrane (Bio-Rad Laboratories, Hercules, Calif.) for 2 hrs at 400 mA, and UV cross-linked. Radiolabeled probes for specific small RNAs were made by end-labeling synthetic DNA oligos (IDT, Coralville, Iowa) with γ-³²P-dATP using T4 polynucleotide kinase (USB, Cleveland, Ohio). Blots were prehybridized and hybridized using ULTRAhyb-Oligo buffer (Ambion, Austin, Tex.). Blots were washed at 42° C. with 2×SSC/0.5% SDS. All blots shown are representative of at least two independent experiments. Locked nucleic acid (LNA) probes were used as indicated in the figure legends; these probes were used when the hybridization signal was not detectable using regular oligonucleotides. LNA oligos were obtained from Sigma-Proligo (St. Louis, Mo.). Hybridization conditions were as described.

MPSS and 454 data generation and analysis. All MPSS sequencing and analysis was performed essentially as described. The small RNA libraries were constructed as previously described. The raw and normalized MPSS data are available at http://mpss.udel.edu/at. 454 analysis was performed essentially as described. Adapter sequences were identified and removed using local alignments. The summary statistics of the rdr2 and wildtype 454 libraries are described in the text; the dcl1-7 and rdr6 libraries included 12,060 and 16,856 adapter-trimmed small RNA inserts, respectively, and the dcl2/3/4 triple mutant 454 library has recently been described.

MPSS signatures were compared to the TIGR annotation version 5.0 and assigned signatures to each location at which a perfect match was found. The number of matches was recorded as the “hits”. As previously described, we merged the MPSS sequencing runs and calculated a single abundance normalized to “transcripts per quarter million” (TPQ) after the removal of rRNAs, tRNAs, snoRNAs, or snRNAs signatures. Clustering of small RNAs was based on the previously described proximity-based algorithm, with the same setting of a 500 bp window for the clusters that was used in our prior analysis. Repeat analysis was also performed as described previously using a combination of programs including RepeatMasker (http://www.repeatmasker.org/), Einverted and Etandem.

A proximity-based algorithm to clusters of small RNA was developed. The clusters were dependent on only the distance between small RNAs and were independent of annotated genomic features such as genes. This facilitated the comparison of clusters across libraries while removing the bias that the annotation might introduce. The optimal cluster size was determined by comparing the results of clustering based on joining signatures within 100, 250 or 500 bp of each other for each library (Table 17A and 17B). Clusters joining small RNAs within 500 bp of each other were used because this size reduced the number of single, unclustered signatures by approximately two-thirds in each library. The exceptionally high average abundance for certain cluster sizes was due to several specific small RNAs such as miRNAs with high abundances. Based on the number of distinct small RNAs contained within each cluster and not the abundance of the signatures, the clusters were then classified in the arbitrarily assigned categories of sparse (1 to 10 signatures), moderate (11 to 25 signatures), or dense (more than 25 signatures).

TABLE 17
Determination of optimal cluster size for small RNA analysis.
A. Inflorescence library^{a}.

| distinct | 100 bp | | | 250 bp | | | 500 bp | | |
|---|---|---|---|---|---|---|---|---|---|
| sigs in | | sig/100 bp | TPQ/sig | | sig/100 bp | TPQ/sig | | sig/100 bp | TPQ/sig |
| clusters | # clusters | avg (std) | avg (std) | # clusters | avg (std) | avg (std) | # clusters | avg (std) | avg (std) |
| 1 | 23,226 | 6 (0) | 3 (9) | 12,589 | 6 (0) | 3 (10) | 7,341 | 6 (0) | 4 (13) |
| 2 | 7,698 | 8 (11) | 4 (13) | 4,402 | 6 (9) | 4 (18) | 2,696 | 5 (9) | 5 (22) |
| 3 | 4,068 | 7 (6) | 4 (14) | 2,509 | 5 (6) | 4 (18) | 1,608 | 4 (5) | 4 (23) |
| 4 | 2,327 | 8 (7) | 5 (27) | 1,641 | 4 (6) | 5 (29) | 1,133 | 3 (5) | 6 (35) |
| 5 | 1,625 | 8 (7) | 7 (85) | 1,173 | 5 (6) | 8 (98) | 857 | 4 (6) | 9 (114) |
| 6 | 1,134 | 9 (9) | 4 (8 ) | 792 | 5 (6) | 4 (6) | 590 | 3 (5) | 4 (7) |
| 7 | 908 | 9 (8) | 23 (402) | 711 | 5 (6) | 28 (453) | 521 | 4 (5) | 37 (529) |
| 8 | 627 | 8 (7) | 21 (308) | 530 | 4 (5) | 22 (334) | 398 | 3 (4) | 9 (88) |
| 9 | 535 | 9 (8) | 9 (68) | 468 | 5 (6) | 9 (72) | 329 | 4 (6) | 32 (376) |
| 10 | 446 | 9 (7) | 8 (66) | 375 | 5 (5) | 9 (77) | 320 | 4 (5) | 10 (83) |
| 11 | 447 | 11 (10) | 9 (56) | 332 | 6 (8) | 9 (65) | 252 | 4 (6) | 10 (74) |
| 12 | 313 | 11 (9) | 13 (79) | 297 | 6 (7) | 12 (81) | 218 | 4 (5) | 12 (79) |
| 13 | 304 | 10 (9) | 5 (9) | 254 | 6 (8) | 4 (3) | 189 | 3 (5) | 8 (51) |
| 14 | 231 | 10 (8) | 6 (15) | 203 | 6 (7) | 6 (15) | 151 | 4 (5) | 6 (17) |
| 15 | 239 | 10 (8) | 5 (3) | 190 | 6 (7) | 6 (24) | 169 | 4 (6) | 6 (25) |
| 16 | 219 | 11 (11) | 6 (14) | 194 | 8 (11) | 8 (32) | 139 | 4 (7) | 8 (37) |
| 17 | 178 | 10 (8) | 6 (9) | 170 | 7 (8) | 5 (9) | 126 | 4 (5) | 4 (2) |
| 18 | 198 | 10 (7) | 7 (12) | 150 | 6 (5) | 5 (4) | 119 | 4 (6) | 5 (5) |
| 19 | 158 | 10 (7) | 8 (14) | 137 | 6 (5) | 5 (9) | 99 | 4 (4) | 4 (2) |
| 20 | 147 | 11 (7) | 8 (16) | 139 | 6 (6) | 7 (14) | 97 | 4 (5) | 6 (14) |
| 21 | 106 | 9 (5) | 7 (15) | 112 | 5 (3) | 7 (15) | 84 | 4 (3) | 7 (14) |
| 22 | 106 | 10 (7) | 6 (9) | 111 | 6 (6) | 5 (9) | 90 | 4 (6) | 6 (10) |
| 23 | 106 | 10 (6) | 6 (12) | 98 | 6 (6) | 6 (12) | 84 | 4 (4) | 8 (18) |
| 24 | 91 | 9 (6) | 6 (4) | 76 | 6 (6) | 4 (2) | 84 | 4 (6) | 4 (3) |
| 25 | 83 | 12 (10) | 8 (15) | 81 | 7 (8) | 7 (14) | 70 | 4 (5) | 7 (13) |
| 26 | 75 | 13 (11) | 12 (21) | 78 | 9 (11) | 11 (19) | 70 | 5 (6) | 9 (18) |
| 27 | 63 | 12 (11) | 9 (17) | 72 | 8 (8) | 9 (16) | 78 | 5 (7) | 7 (14) |
| 28 | 65 | 11 (10) | 8 (15) | 59 | 8 (5) | 7 (12) | 64 | 5 (5) | 7 (12) |
| 29 | 64 | 12 (8) | 9 (12) | 64 | 8 (7) | 7 (12) | 53 | 5 (4) | 7 (13) |
| 30 | 56 | 11 (7) | 10 (15) | 58 | 7 (6) | 8 (14) | 56 | 5 (5) | 4 (2) |
| >30 | 1,154 | 13 (7) | 8 (9) | 1,180 | 8 (7) | 7 (10) | 1,302 | 5 (5) | 6 (10) |
| TOTAL | 46,997 | | | 29,245 | | | 19,387 | | |

B. Seedling library^{b}.

| distinct | 100 bp | | | 250 bp | | | 500 bp | | |
|---|---|---|---|---|---|---|---|---|---|
| sigs in | | sig/100 bp | TPM/sig | | sig/100 bp | TPM/sig | | sig/100 bp | TPM/sig |
| clusters | # clusters | avg (std) | avg (std) | # clusters | avg (std) | avg (std) | # clusters | avg (std) | avg (std) |
| 1 | 15,302 | 6 (0) | 6 (38) | 9,097 | 6 (0) | 6 (48) | 5,810 | 6 (0) | 7 (60) |
| 2 | 4,900 | 8 (8) | 6 (16) | 3,261 | 5 (7) | 6 (17) | 2,148 | 5 (7) | 7 (20) |
| 3 | 2,271 | 8 (7) | 8 (34) | 1,666 | 5 (7) | 8 (40) | 1,169 | 4 (7) | 9 (48) |
| 4 | 1,226 | 8 (7) | 23 (227) | 1,072 | 6 (7) | 19 (232) | 733 | 4 (7) | 24 (281) |
| 5 | 752 | 9 (8) | 38 (673) | 727 | 5 (6) | 38 (685) | 536 | 4 (6) | 13 (89) |
| 6 | 536 | 10 (8) | 55 (686) | 491 | 6 (7) | 58 (716) | 398 | 4 (7) | 69 (795) |
| 7 | 390 | 11 (10) | 70 (712) | 363 | 6 (8) | 50 (693) | 274 | 4 (7) | 109 (1121) |
| 8 | 314 | 12 (9) | 13 (40) | 320 | 7 (8) | 17 (96) | 248 | 5 (7) | 18 (109) |
| 9 | 267 | 11 (9) | 20 (49) | 265 | 7 (7) | 30 (209) | 214 | 4 (5) | 28 (232) |
| 10 | 193 | 12 (10) | 21 (98) | 209 | 8 (9) | 24 (134) | 173 | 5 (6) | 25 (147) |
| 11 | 164 | 13 (11) | 28 (208) | 197 | 7 (10) | 23 (190) | 144 | 5 (7) | 26 (222) |
| 12 | 145 | 12 (11) | 11 (8) | 146 | 7 (9) | 10 (8) | 136 | 4 (5) | 9 (8) |
| 13 | 124 | 12 (10) | 15 (16) | 113 | 7 (9) | 12 (12) | 127 | 3 (5) | 9 (7) |
| 14 | 110 | 10 (9) | 12 (10) | 111 | 5 (5) | 10 (7) | 109 | 4 (4) | 9 (5) |
| 15 | 105 | 14 (12) | 14 (11) | 98 | 8 (10) | 12 (13) | 95 | 5 (7) | 9 (7) |
| 16 | 79 | 17 (15) | 16 (15) | 88 | 7 (7) | 22 (73) | 93 | 5 (6) | 20 (71) |
| 17 | 81 | 12 (8) | 15 (13) | 84 | 7 (8) | 12 (11) | 86 | 5 (8) | 10 (8) |
| 18 | 52 | 15 (15) | 16 (16) | 62 | 6 (8) | 21 (57) | 66 | 4 (7) | 18 (54) |
| 19 | 69 | 15 (16) | 15 (14) | 67 | 7 (6) | 11 (9) | 65 | 4 (4) | 11 (12) |
| 20 | 53 | 14 (12) | 20 (16) | 62 | 7 (8) | 13 (9) | 64 | 3 (3) | 10 (7) |
| 21 | 50 | 16 (14) | 19 (16) | 61 | 10 (10) | 19 (17) | 61 | 6 (7) | 13 (9) |
| 22 | 45 | 17 (16) | 22 (21) | 61 | 8 (10) | 16 (18) | 47 | 5 (6) | 15 (17) |
| 23 | 41 | 13 (11) | 22 (19) | 47 | 8 (10) | 16 (17) | 59 | 4 (4) | 12 (11) |
| 24 | 23 | 14 (10) | 17 (14) | 33 | 6 (5) | 13 (15) | 31 | 5 (5) | 14 (16) |
| 25 | 31 | 19 (13) | 23 (20) | 52 | 8 (10) | 16 (17) | 44 | 6 (8) | 15 (17) |
| 26 | 18 | 16 (12) | 26 (21) | 42 | 7 (10) | 16 (15) | 42 | 6 (10) | 14 (15) |
| 27 | 24 | 18 (15) | 22 (17) | 29 | 6 (5) | 13 (14) | 32 | 4 (4) | 13 (13) |
| 28 | 27 | 15 (13) | 25 (18) | 28 | 9 (9) | 17 (14) | 30 | 4 (4) | 16 (17) |
| 29 | 26 | 36 (28) | 32 (16) | 22 | 9 (11) | 17 (15) | 26 | 5 (5) | 14 (13) |
| 30 | 18 | 23 (20) | 31 (14) | 23 | 7 (6) | 15 (15) | 22 | 3 (3) | 12 (11) |
| >30 | 457 | 21 (14) | 31 (12) | 441 | 14 (10) | 25 (14) | 413 | 5 (6) | 14 (12) |
| TOTAL | 27,893 | | | 19,338 | | | 13,495 | | |

Excludes clusters containing any signatures matching to annotated rRNAs, tRNAs, snoRNAs, and snRNAs.
^{a}Includes 239,745 distinct signatures.
^{b}Includes 106,088 distinct signatures.

Repeats in the Arabidopsis genome were identified using a combination of programs. For the identification of transposons and retrotransposons, we utilized a dataset comprised of those sequences annotated by TIGR (version 5.0) augmented with the results of RepeatMasker™. For tandem and inverted repeats, we used the programs Einverted and Etandem.

While most Arabidopsis miRNAs have been identified by traditional cloning and sequencing of small RNAs, it is unlikely that these screens are saturating for rare or tissue-specific miRNAs. The need for additional methods of miRNA identification led to the development of bioinformatics methods for the prediction of miRNAs. Most of these computer algorithms rely on evolutionary conservation of miRNA sequences between different species, and therefore are limited to the detection of only conserved miRNAs, although at least one analysis has relied only on intra-genomic comparisons. Even these predictions ultimately require either high-throughput or highly sensitive methods for validation. With MPSS and other high-throughput sequencing technologies, the sequencing of small RNAs is no longer a limiting factor in the discovery of novel miRNAs. However, by combining these approaches with mutants in which miRNAs are significantly enriched compared with wild type, such as rdr2 and dcl2/3/4, we can efficiently delineate the small RNAs as miRNAs, siRNAs, or other categories. Even at relatively low sampling depths, many known miRNAs were observed and their abundance was measured. Compared to wildtype, the MPSS data for rdr2 was dramatically simplified and “cleaned up” of siRNAs, making miRNA candidates much easier to identify. 454 analysis indicated that the rdr2 and dcl2/3/4 triple mutants are most similar in their small RNA profiles, consistent with the idea that these genes may be in the same pathway involved in heterochromatic siRNA production and a mutant of either type (rdr2 and dcl2/3/4) enriches for miRNAs.

The following references are incorporated herein by reference in their entirety.

### REFERENCES

1. Bartel, B. & Bartel, D. P. MicroRNAs: at the root of plant development? Plant Physiol 132, 709-17 (2003).
2. Carrington, 3. C. & Ambros, V. Role of microRNAs in plant and animal development. Science 301, 336-8 (2003).
3. Meister, G. & Tuschl, T. Mechanisms of gene silencing by double-stranded RNA. Nature 431, 343-9 (2004).
4. Baulcombe, D. RNA silencing in plants. Nature 431, 356-63 (2004).
5. Bernstein, E., Caudy, A. A., Hammond, S. M. & Hannon, G. J. Role for a bidentate ribonuclease in the initiation step of RNA interference. Nature 409, 363-6 (2001).
6. Grishok, A. et al. Genes and mechanisms related to RNA interference regulate expression of the small temporal RNAs that control C. elegans developmental timing. Cell 106, 23-34 (2001).
7. Ketting, R. F. et al. Dicer functions in RNA interference and in synthesis of small RNA involved in developmental timing in C. elegans. Genes Dev 15, 2654-9 (2001).
8. Hutvagner, G. et al. A cellular function for the RNA-interference enzyme Dicer in the maturation of the let-7 small temporal RNA. Science 293, 834-8 (2001).
9. Lee, Y. S. et al. Distinct roles for Drosophila Dicer-1 and Dicer-2 in the siRNA/miRNA silencing pathways. Cell 117, 69-81 (2004).
10. Mallory, A. C. &Vaucheret, H. MicroRNAs: something important between the genes. Curr Opin Plant Biol 7, 120-5 (2004).
11. Bartel, D. P. MicroRNAs: genomics, biogenesis, mechanism, and function. Cell 116, 281-97 (2004).
12. Xie, Z. et al. Genetic and Functional Diversification of Small RNA Pathways in Plants. PLoS Biol 2, E104 (2004).
13. Hannon, G. J. RNA interference. Nature 418, 244-51 (2002).
14. Verdel, A. et al. RNAi-mediated targeting of heterochromatin by the RITS complex. Science 303, 672-6 (2004).
15. Schwarz, D. S. et al. Asymmetry in the assembly of the RNAI enzyme complex. Cell 115, 199-208 (2003).
16. Tang, G., Reinhart, B. J., Bartel, D. P. & Zamore, P. D. A biochemical framework for RNA silencing in plants. Genes Dev 17, 49-63 (2003).
17. Llave, C., Xie, Z., Kasschau, K. D. & Carrington, J. C. Cleavage of Scarecrow-like mRNA targets directed by a class of Arabidopsis miRNA. Science 297, 2053-6 (2002).
18. Aukerman, M. J. & Sakai, H. Regulation of flowering time and floral organ identity by a MicroRNA and its APETALA2-like target genes. Plant Cell 15, 2730-41 (2003).
19. Chen, X. A microRNA as a translational repressor of APETALA2 in Arabidopsis flower development. Science 303, 2022-5 (2004).
20. Vella, M. C., Choi, E. Y., Lin, S. Y., Reinert, K. & Slack, F. J. The C. elegans microRNA let-7 binds to imperfect let-7 complementary sites from the lin-41 3′UTR. Genes Dev 18, 132-7 (2004).
21. Reinhart, B. I., Weinstein, E. G., Rhoades, M. W., Bartel, B. & Bartel, D. P. MicroRNAs in plants. Genes Dev. 16, 1616-1626 (2002).
22. Aravin, A. A. et al. The small RNA profile during Drosophila melanogaster development. Dev Cell 5, 337-50 (2003).
23. Lagos-Quintana, M., Rauhut, R., Lendeckel, W. & Tuschl, T. Identification of novel genes coding for small expressed RNAs. Science 294, 853-8 (2001).
24. Llave, C., Kasschau, K. D., Rector, M. A. & Carrington, I. C. Endogenous and silencing-associated small RNAs in plants. Plant Cell 14, 1605-19 (2002).
25. Krichevsky, A. M., King, K. S., Donahue, C. P., Khrapko, K. & Kosik, K. S. A microRNA array reveals extensive regulation of microRNAs during brain development. Rna 9, 1274-81 (2003).
26. Barad, O. et al. MicroRNA expression detected by oligonucleotide microarrays: system establishment and expression profiling in human tissues. Genome Res 14, 2486-94 (2004).
27. Babak, T., Zhang, W., Morris, Q., Blencowe, B. J. & Hughes, T. R. Probing microRNAs with microarrays: tissue specificity and functional inference. Rna 10, 1813-9 (2004).
28. Allawi, H. T. et al. Quantitation of microRNAs using a modified Invader assay. Rna 10, 1153-61 (2004).
29. Brenner, S. et al. Gene expression analysis by massively parallel signature sequencing (MPSS) on microbead arrays. Nat Biotechnol 18, 630-4 (2000).
30. Hamilton, A. J. & Baulcombe, D. C. A species of small antisense RNA in posttranscriptional gene silencing in plants. Science 286, 950-2 (1999).
31. Brenner, S. et al. In vitro cloning of complex mixtures of DNA on microbeads: physical separation of differentially expressed cDNAs. Proc Natl Acad Sci USA 97, 1665-70 (2000).
32. Wortman, 1. R. et al. Annotation of the Arabidopsis genome. Plant Physiol 132, 461-8 (2003).
33. Meyers, B. C. et al. The Use of MPSS for Whole-Genome Transcriptional Analysis in Arabidopsis. Genome Res 14, 1641-53 (2004).
34. Park, W., Li, J., Song, R., Messing, J. & Chen, X. CARPEL FACTORY, a Dicer homolog, and HEN1, a novel protein, act in microRNA metabolism in Arabidopsis thaliana. Curr Biol 12, 1484-95 (2002).
35. Sunkar, R. & Zhu, J. K. Novel and stress-regulated microRNAs and other small RNAs from Arabidopsis. Plant Cell 16, 2001-19 (2004).
36. Sijen, T. & Plasterk, R. H. Transposon silencing in the Caenorhabditis elegans germ line by natural RNAi. Nature 426, 310-4 (2003).
37. Lippman, Z. & Martienssen, R. The role of RNA interference in heterochromatic silencing. Nature 431, 364-70 (2004).
38. Parizotto, E. A., Dunoyer, P., Rahm, N., Himber, C. & Voinnet, O. In vivo investigation of the transcription, processing, endonucleolytic activity, and functional relevance of the spatial distribution of a plant miRNA. Genes Dev 18, 2237-42 (2004).
39. Zamore, P. D., Tuschl, T., Sharp, P. A. & Bartel, D. P. RNAi: double-stranded RNA directs the ATP-dependent cleavage of mRNA at 21 to 23 nucleotide intervals. Cell 101, 25-33 (2000).
40. Jackson, A. L. & Linsley, P. S. Noise amidst the silence: off-target effects of siRNAs? Trends Genet 20, 521-4 (2004).
41. Lim, L. P. et al. Microarray analysis shows that some microRNAs downregulate large numbers of target mRNAs. Nature 433, 769-73 (2005).
42. Meyers, B. C., Tingey, S. V. & Morgante, M. Abundance, distribution, and transcriptional activity of repetitive elements in the maize genome. Genome Res 11, 1660-76 (2001).
43. Meyers, B. C. et al. Analysis of the transcriptional complexity of Arabidopsis by massively parallel signature sequencing. Nat Biotechnol 22, 1006-1011 (2004).
44. Melquist, S., Luff, B. & Bender, J. Arabidopsis PAI gene arrangements, cytosine methylation and expression. Genetics 153, 401-13 (1999).
45. Yamada, K. et al. Empirical analysis of transcriptional activity in the Arabidopsis genome. Science 302, 842-6 (2003).
46. Eszterhas, S. K., Bouhassira, E. E., Martin, D. I. & Fiering, S. Transcriptional interference by independently regulated genes occurs in any relative arrangement of the genes and is influenced by chromosomal integration position. Mol Cell Biol 22, 469-79 (2002).
47. Ambros, V. et al. A uniform system for microRNA annotation. RNA 9, 277-9 (2003).
48. Lim, L. P. et al. The microRNAs of Caenorhabditis elegans. Genes Dev 17, 991-1008 (2003).
49. Lai, E. C., Tomancak, P., Williams, R. W. & Rubin, G. M. Computational identification of Drosophila microRNA genes. Genome Biol 4, R42 (2003).
50. Adai, A. et al. Computational prediction of miRNAs in Arabidopsis thaliana. Genome Res 15, 78-91 (2005).
51. Jones-Rhoades, M. W. & Bartel, D. P. Computational identification of plant microRNAs and their targets, including a stress-induced miRNA. Mol Cell 14, 787-99 (2004).
52. Bonnet, E., Wuyts, J., Rouze, P. &Van de Peer, Y. Detection of 91 potential conserved plant microRNAs in Arabidopsis thaliana and Oryza sativa identifies important target genes. Proc Natl Acad Sci USA 101, 11511-6 (2004).
53. Ruvkun, G. Molecular biology. Glimpses of a tiny RNA world. Science 294, 797-9 (2001).
54. Peragine, A., Yoshikawa, M., Wu, G., Albrecht, H. L. & Poethig, R. S. SGS3 and SGS2/SDE1/RDR6 are required for juvenile development and the production of trans-acting siRNAs in Arabidopsis. Genes Dev 18, 2368-79 (2004).
55. Vazquez, F. et al. Endogenous trans-acting siRNAs regulate the accumulation of Arabidopsis mRNAs. Mol Cell 16, 69-79 (2004).
56. Rice, P., Longden, I. & Bleasby, A. EMBOSS: the European Molecular Biology Open Software Suite. Trends Genet 16, 276-7 (2000).
57. Jones-Rhoades, M. W. & Bartel, D. P. Computational identification of plant microRNAs and their targets, including a stress-induced miRNA. Mol Cell 14, 787-99 (2004).
58. Adai, A., C. Johnson, S. Mlotshwa, S. Archer-Evans, V. Manocha, V. Vance, and V. Sundaresan. 2005. Computational prediction of miRNAs in Arabidopsis thaliana. Genome Res 15: 78-91.
59. Allen, E., Z. Xie, A. M. Gustafson, and J. C. Carrington. 2005. microRNA-directed phasing during trans-acting siRNA biogenesis in plants. Cell 121: 207-221.
60. Allen, E., Z. Xie, A. M. Gustafson, G. H. Sung, J. W. Spatafora, and J. C. Carrington. 2004. Evolution of microRNA genes by inverted duplication of target gene sequences in Arabidopsis thaliana. Nat Genet 36: 1282-1290.
61. Ambros, V., B. Bartel, D. P. Bartel, C. B. Burge, J. C. Carrington, X. Chen, G. Dreyfuss, S. R. Eddy, S. Griffiths-Jones, M. Marshall, M. Matzke, G. Ruvkun, and T. Tuschl. 2003. A uniform system for microRNA annotation. RNA 9: 277-279.
62. Arazi, T., M. Talmor-Neiman, R. Stav, M. Riese, P. Huijser, and D. C. Baulcombe. 2005. Cloning and characterization of micro-RNAs from moss. Plant J 43: 837-848.
63. Axtell, M. J. and D. P. Bartel. 2005. Antiquity of microRNAs and their targets in land plants. Plant Cell 17: 1658-1673.
64. Bentwich, I., A. Avniel, Y. Karov, R. Aharonov, S. Gilad, O. Barad, A. Barzilai, P. Einat, U. Einav, E. Meiri, E. Sharon, Y. Spector, and Z. Bentwich. 2005. Identification of hundreds of conserved and nonconserved human microRNAs. Nat Genet37: 766-770.
65. Bonnet, E., J. Wuyts, P. Rouze, and Y. Van de Peer. 2004. Detection of 91 potential conserved plant microRNAs in Arabidopsis thaliana and Oryza sativa identifies important target genes. Proc Natl Acad Sci USA 101: 11511-11516.
66. Borsani, O., J. Zhu, P. E. Verslues, R. Sunkar, and J. K. Zhu. 2005. Endogenous siRNAs Derived from a Pair of Natural cis-Antisense Transcripts Regulate Salt Tolerance in Arabidopsis. Cell 123: 1279-1291.
67. Brenner, S., M. Johnson, J. Bridgham, G. Golda, D. H. Lloyd, D. Johnson, S. Luo, S. McCurdy, M. Foy, M. Ewan, R. Roth, D. George, S. Eletr, G. Albrecht, E. Vermaas, S. R. Williams, K. Moon, T. Burcham, M. Pallas, R. B. DuBridge, J. Kirchner, K. Fearon, J. Mao, and K. Corcoran. 2000a. Gene expression analysis by massively parallel signature sequencing (MPSS) on microbead arrays. Nat Biotechnol 18: 630-634.
68. Brenner, S., S. R. Williams, E. H. Vermaas, T. Storck, K. Moon, C. McCollum, J. I. Mao, S. Luo, J. J. Kirchner, S. Eletr, R. B. DuBridge, T. Burcham, and G. Albrecht. 2000b. In vitro cloning of complex mixtures of DNA on microbeads: physical separation of differentially expressed cDNAs. Proc NatlAcad Sci USA 97:1665-1670.
69. Brodersen, P. and O. Voinnet. 2006. The diversity of RNA silencing pathways in plants. Trends Genet In Press.
70. Chen, X. 2005. microRNA biogenesis and function in plants. FEBS Lett 579: 5923-5931.
71. Gasciolli, V., A. C. Mallory, D. P. Bartel, and H. Vaucheret. 2005. Partially redundant functions of Arabidopsis DICER-like enzymes and a role for DCL4 in producing trans-acting siRNAs. Curr Biol 15: 1494-1500.
72. Grad, Y., J. Aach, G. D. Hayes, B. J. Reinhart, G. M. Church, G. Ruvkun, and J. Kim. 2003. Computational and experimental identification of C. elegans microRNAs. Mol Cell 11: 1253-1263.
73. Grundhoff, A., C. S. Sullivan, and D. Ganem. 2006. A combined computational and microarray-based approach identifies novel microRNAs encoded by human gamma-herpesviruses. RNA 12: 733-750.
74. Gustafson, A. M., E. Allen, S. Givan, D. Smith, J. C. Carrington, and K. D. Kasschau. 2005. ASRP: the Arabidopsis Small RNA Project Database. Nucleic Acids Res 33: D637-640.
75. Henderson, I. R., X. Zhang, C. Lu, L. Johnson, B. C. Meyers, P. J. Green, and S. E. Jacobsen. 2006. Dissecting Arabidopsis DICER function in small RNA processing, gene silencing, and DNA methylation patterning. Nat Genet In press.
76. Jones-Rhoades, M. W. and D. P. Bartel. 2004. Computational identification of plant microRNAs and their targets, including a stress-induced miRNA. Mol Cell 14: 787-799.
77. Jones-Rhoades, M. W., D. P. Bartel, and B. Bartel. 2006. MicroRNAs and their regulatory roles in plants. Annu Rev Plant Biol 57: 19-53.
78. Kasschau, K. D., Z. Xie, E. Allen, C. Llave, E. J. Chapman, K. A. Krizan, and J. C. Carrington. 2003. P1/HC-Pro, a viral suppressor of RNA silencing, interferes with Arabidopsis development and miRNA function. Dev Cell 4: 205-217.
79. Kurihara, Y., Y. Takashi, and Y. Watanabe. 2006. The interaction between DCL1 and HYL1 is important for efficient and precise processing of pri-miRNA in plant microRNA biogenesis. RNA 12: 206-212.
80. Kusaba, M., K. Dwyer, J. Hendershot, J. Vrebalov, J. B. Nasrallah, and M. E. Nasrallah. 2001. Self-incompatibility in the genus Arabidopsis: characterization of the S locus in the outcrossing A. lyrata and its autogamous relative A. thaliana. Plant Cell 13: 627-643.
81. Lippman, Z. and R. Martienssen. 2004. The role of RNA interference in heterochromatic silencing. Nature 431: 364-370.
82. Llave, C., K. D. Kasschau, M. A. Rector, and J. C. Carrington. 2002a. Endogenous and silencing-associated small RNAs in plants. Plant Cell 14: 1605-1619.
83. Llave, C., Z. Xie, K. D. Kasschau, and J. C. Carrington. 2002b. Cleavage of Scarecrow-like mRNA targets directed by a class of Arabidopsis miRNA. Science 297: 2053-2056.
84. Lu, C., S. S. Tej, S. Luo, C. D. Haudenschild, B. C. Meyers, and P. J. Green. 2005. Elucidation of the small RNA component of the transcriptome. Science 309: 1567-1569.
85. Margulies, M., M. Egholm, W. E. Altman, S. Attiya, J. S. Bader, L. A. Bemben, J. Berka, M. S. Braverman, Y. J. Chen, Z. Chen, S. B. Dewell, L. Du, J. M. Fierro, X. V. Gomes, B. C. Godwin, W. He, S. Helgesen, C. H. Ho, G. P. Irzyk, S. C. Jando, M. L. Alenquer, T. P. Jarvie, K. B. Jirage, J. B. Kim, J. R. Knight, J. R. Lanza, J. H. Leamon, S. M. Lefkowitz, M. Lei, J. Li, K. L. Lohman, H. Lu, V. B. Makhijani, K. E. McDade, M. P. McKenna, E. W. Myers, E. Nickerson, J. R. Nobile, R. Plant, B. P. Puc, M. T. Ronan, G. T. Roth, G. J. Sarkis, J. F. Simons, J. W. Simpson, M. Srinivasan, K. R. Tartaro, A. Tomasz, K. A. Vogt, G. A. Volkmer, S. H. Wang, Y. Wang, M. P. Weiner, P. Yu, R. F. Begley, and J. M. Rothberg. 2005. Genome sequencing in microfabricated high-density picolitre reactors. Nature 437: 376-380.
86. Martienssen, R. A. 2003. Maintenance of heterochromatin by RNA interference of tandem repeats. Nat Genet 35: 213-214.
87. May, B. P., Z. B. Lippman, Y. Fang, D. L. Spector, and R. A. Martienssen. 2005. Differential Regulation of Strand-Specific Transcripts from Arabidopsis Centromeric Satellite Repeats. PLoS Genet 1: e79.
88. Meyers, B. C., A. Kozik, A. Griego, H. Kuang, and R. W. Michelmore. 2003. Genome-wide analysis of NBS-LRR-encoding genes in Arabidopsis. Plant Cell 15: 809-834.
89. Meyers, B. C., F. F. Souret, C. Lu, and P. J. Green. 2006. Sweating the small stuff: microRNA discovery in plants. Curr Opin Biotechnol 17: 139-146.
90. Meyers, B. C., S. S. Tej, T. H. Vu, C. D. Haudenschild, V. Agrawal, S. B. Edberg, H. Ghazal, and S. Decola. 2004. The use of MPSS for whole-genome transcriptional analysis in Arabidopsis. Genome Res 14:1641-1653.
91. Nasrallah, M. E., P. Liu, and J. B. Nasrallah. 2002. Generation of self-incompatible Arabidopsis thaliana by transfer of two S locus genes from A. lyrata. Science 297: 247-249.
92. Naumann, K., A. Fischer, I Hofmann, V. Krauss, S. Phalke, K. Irmler, G. Hause, A. C. Aurich, R. Dorn, T. Jenuwein, and G. Reuter. 2005. Pivotal role of AtSUVH2 in heterochromatic histone methylation and gene silencing in Arabidopsis. EMBO J. 24: 1418-1429.
93. Park, W., J. Li, R. Song, J. Messing, and X. Chen. 2002. CARPEL FACTORY, a Dicer homolog, and HEN1, a novel protein, act in microRNA metabolism in Arabidopsis thaliana. Curr Biol 12: 1484-1495.
94. Peragine, A., M. Yoshikawa, G. Wu, H. L. Albrecht, and R. S. Poethig. 2004. SGS3 and SGS2/SDE1/RDR6 are required for juvenile development and the production of trans-acting siRNAs in Arabidopsis. Genes Dev 18: 2368-2379.
95. Redei, G. P. 1975. Arabidopsis as a genetic tool. Annu Rev Genet 9: 111-127.
96. Reinhart, B. J., E. G. Weinstein, M. W. Rhoades, B. Bartel, and D. P. Bartel. 2002. MicroRNAs in plants. Genes Dev. 16:1616-1626.
97. Rhoades, M. W., B. J. Reinhart, L. P. Lim, C. B. Burge, B. Bartel, and D. P. Bartel. 2002. Prediction of plant microRNA targets. Cell 110: 513-520.
98. Rice, P., I. Longden, and A. Bleasby. 2000. EMBOSS: the European Molecular Biology Open Software Suite. Trends Genet 16: 276-277.
99. Sunkar, R. and J. K. Zhu. 2004. Novel and stress-regulated microRNAs and other small RNAs from Arabidopsis. Plant Cell 16: 2001-2019.
100. Valoczi, A., C. Hornyik, N. Varga, J. Burgyan, S. Kauppinen, and Z. Havelda. 2004. Sensitive and specific detection of microRNAs by northern blot analysis using LNA-modified oligonucleotide probes. Nucleic Acids Res 32: el 75.
101. Vaucheret, H. 2006. Post-transcriptional small RNA pathways in plants: mechanisms and regulations. Genes Dev 20: 759-771.
102. Vaucheret, H., A. C. Mallory, and D. P. Bartel. 2006. AGO1 homeostasis entails coexpression of MIR168 and AGO1 and preferential stabilization of miR168 by AGO1. Mol Cell 22: 129-136.
103. Vaucheret, H., F. Vazquez, P. Crete, and D. P. Bartel. 2004. The action of ARGONAUTE1 in the miRNA pathway and its regulation by the miRNA pathway are crucial for plant development. Genes Dev 18: 1187-1197.
104. Vazquez, F., H. Vaucheret, R. Rajagopalan, C. Lepers, V. Gasciolli, A. C. Mallory, J. L. Hilbert, D. P. Bartel, and P. Crete. 2004. Endogenous trans-acting siRNAs regulate the accumulation of Arabidopsis mRNAs. Mol Cell 16: 69-79.
105. Wang, X. J., J. L. Reyes, N. H. Chua, and T. Gaasterland. 2004. Prediction and identification of Arabidopsis thaliana microRNAs and their mRNA targets. Genome Biol 5: R65.
106. Wassenegger, M. and G. Krczal. 2006. Nomenclature and functions of RNA-directed RNA polymerases. Trends Plant Sci 11: 142-151.

107. Wortman, J. R., B. J. Haas, L. I. Hannick, R. K. Smith, Jr., R. Maiti, C. M. Ronning, A. P. Chan, C. Yu, M. Ayele, C. A. Whitelaw, O. R. White, and C. D. Town. 2003. Annotation of the Arabidopsis genome. Plant Physiol 132: 461-468.
108. Xie, Z., E. Allen, N. Fahlgren, A. Calamar, S. A. Givan, and J. C. Carrington. 2005a. Expression of Arabidopsis MIRNA Genes. Plant Physiol 138: 2145-2154.
109. Xie, Z., E. Allen, A. Wilken, and J. C. Carrington. 2005b. DICER-LIKE 4 functions in trans-acting small interfering RNA biogenesis and vegetative phase change in Arabidopsis thaliana. Proc Natl Acad Sci USA 102:12984-12989.
110. Xie, Z., L. K. Johansen, A. M. Gustafson, K. D. Kasschau, A. D. Lellis, D. Zilberman, S. E. Jacobsen, and J. C. Carrington. 2004. Genetic and functional diversification of small RNA pathways in plants. PLoS Biol 2: E104.
111. Xie, Z., K. D. Kasschau, and J. C. Carrington. 2003. Negative feedback regulation of Dicer-Like1 in Arabidopsis by microRNA-guided mRNA degradation. Curr Biol 13: 784-789.
112. Yoshikawa, M., A. Peragine, M. Y. Park, and R. S. Poethig. 2005. A pathway for the biogenesis of trans-acting siRNAs in Arabidopsis. Genes Dev 19: 2164-2175.
113. Yu, D., B. Fan, S. A. MacFarlane, and Z. Chen. 2003. Analysis of the involvement of an inducible Arabidopsis RNA-dependent RNA polymerase in antiviral defense. Mol Plant Microbe Interact 16: 206-216.
114. Zhang, B., X. Pan, C. H. Cannon, G. P. Cobb, and T. A. Anderson. 2006. Conservation and divergence of plant microRNA genes. Plant J 46: 243-259.

What is claimed:

1. A method of identifying a full length small RNA from a signature sequence RNA molecule comprising:
a. providing a genomic DNA database;
b. identifying said signature sequence of said small RNA molecule from said database using MPSS method, wherein said signature sequence comprises a portion of a full sequence of said small RNA molecule, wherein said small RNA molecule comprises about 15 to about 30 nucleotides;
c. comparing said signature sequence to said genomic database;
d. identifying one or more genomic regions that indicate identity with said signature sequence; and
e. extending said signature sequence by a necessary number of nucleotides to obtain said full sequence of said small RNA molecule.

2. The method of claim 1 wherein said small RNA signature sequence comprises about 15 to about 20 nucleotides.

3. The method of claim 1 wherein said signature sequence is selected from the group consisting of SEQ ID NOs: 1-185,396.

4. The method of claim 1 wherein said signature sequence is extended by from about 1 to about 13 nucleotides.

5. The method of claim 1 wherein said signature sequence is extended in a 3′ direction.

6. The method of claim 1 wherein said signature sequence is extended in a 5′ direction.

7. The method of claim 1 wherein the small RNA molecule comprises a small interfering RNA or a microRNA.

8. A library of small RNA signature sequences from Arabidopsis thaliana comprising a plurality of sequences selected from the group consisting of SEQ ID NOs: 1-185,396.

9. The library of claim 8 wherein said plurality of sequences comprises all of SEQ ID NOs: 1-185,396.

10. An isolated small RNA molecule comprising a nucleic acid sequence having from about 15 to about 30 nucleotides, wherein the nucleic acid is sufficiently complementary to a plant gene to down-regulate the plant gene by RNA interference.

11. The isolated small RNA molecule of claim 10, wherein the nucleic acid sequence is at least 75% homologous to a member selected from the group consisting of SEQ ID NO. 1-185,413.

12. The isolated small RNA molecule of claim 10, wherein the plant gene is an Arabidopsis thaliana gene.

13. The isolated small RNA molecule of claim 10, wherein the nucleic acid is an siRNA, miRNA or combination thereof.

14. The isolated small RNA molecule of claim 10 wherein:
a) the small RNA molecule that down-regulates expression of an NBS-LRR disease resistance gene via RNA interference (RNAi);
b) the small RNA molecule is from about 15 to about 30 nucleotides in length; and
c) the small RNA molecule comprises a nucleotide sequence having sufficient complementarity to an RNA of said NBS-LRR disease resistance gene for the small RNA molecule to direct cleavage of said RNA via RNAi.

15. The isolated small RNA molecule of claim 14, comprising a nucleic acid having at least 75% homology to SEQ ID NO. 185,398.

16. The isolated small RNA molecule of claim 10 wherein:
a) the small RNA molecule down-regulates expression of an DNA (cytosine-5)-methyltransferase gene via RNA interference (RNAi);
b) the small RNA molecule is from about 15 to about 30 nucleotides in length; and
c) the small RNA molecule comprises a nucleotide sequence having sufficient complementarity to an RNA of said DNA (cytosine-5)-methyltransferase gene for the small RNA molecule to direct cleavage of said RNA via RNAi.

17. The isolated small RNA molecule of claim 16, comprising a nucleic acid having at least 75% homology to SEQ ID NO. 185,399.

18. The isolated small RNA molecule of claim 10 wherein:
a) the small RNA molecule down-regulates expression of an F-box family gene via RNA interference (RNAi);
b) the small RNA molecule is from about 15 to about 30 nucleotides in length; and
c) the small RNA molecule comprises a nucleotide sequence having sufficient complementarity to an RNA of said F-box family gene for the small RNA molecule to direct cleavage of said RNA via RNAi.

19. The isolated small RNA molecule of claim 18, comprising a nucleic acid having at least 75% homology to SEQ ID NO. 185,400.

20. The isolated small RNA molecule of claim 10 wherein:
a) the small RNA molecule down-regulates expression of an galactosidyltransferase gene via RNA interference (RNAi), wherein:
b) the small RNA molecule is from about 15 to about 30 nucleotides in length; and
c) the small RNA molecule comprises a nucleotide sequence having sufficient complementarity to an RNA of said galactosidyltransferase gene for the small RNA molecule to direct cleavage of said RNA via RNAi.

21. The isolated small RNA molecule of claim 20, comprising a nucleic acid having at least 75% homology to SEQ ID NO. 185,401.

22. The isolated small RNA molecule of claim 10 wherein:
a) the small RNA molecule down-regulates expression of an SET domain-containing gene via RNA interference (RNAi);
b) the small RNA molecule is from about 15 to about 30 nucleotides in length; and
c) the small RNA molecule comprises a nucleotide sequence having sufficient complementarity to an RNA of said SET domain-containing gene for the small RNA molecule to direct cleavage of said RNA via RNAi.

23. The isolated small RNA molecule of claim 22, comprising a nucleic acid having at least 75% homology to SEQ ID NO. 185,404.

24. The isolated small RNA molecule of claim 10 wherein:
a) the small RNA molecule down-regulates expression of an S-locus protein kinase gene via RNA interference (RNAi);
b) the small RNA molecule is from about 15 to about 30 nucleotides in length; and
c) the small RNA molecule comprises a nucleotide sequence having sufficient complementarity to an RNA of said S-locus protein kinase gene for the small RNA molecule to direct cleavage of said RNA via RNAi.

25. The isolated small RNA molecule of claim 24, comprising a nucleic acid having at least 75% homology to SEQ ID NO. 185,405.

26. The isolated small RNA molecule of claim 10 wherein:
a) the small RNA molecule down-regulates expression of an Extra-large G-Protein-related protein gene via RNA interference (RNAi);
a) the small RNA molecule is from about 15 to about 30 nucleotides in length; and
b) the small RNA molecule comprises a nucleotide sequence having sufficient complementarity to an RNA of said Extra-large G-Protein-related protein gene for the small RNA molecule to direct cleavage of said RNA via RNAi.

27. The isolated small RNA molecule of claim 26, comprising a nucleic acid having at least 75% homology to SEQ ID NO. 185,409.

28. The isolated small RNA molecule of claim 10 wherein
a) the small RNA molecule down-regulates a plant gene comprising a nucleic acid having at least 75% homology to a member selected from the group consisting of SEQ ID NOs. 185,397-185,409; and
b) the nucleic acid is sufficiently complementary to the plant gene to down-regulate the plant gene by RNA interference.

29. An expression vector comprising a nucleic acid sequence encoding a nucleic acid having at least 75% homology to a member selected from the group consisting of SEQ ID NO. 1-185,413, wherein the expression vector comprises a transcription initiation region; a transcription termination region; and wherein said nucleic acid sequence is operably linked to said initiation region and said termination region.

30. The expression vector of claim 29, wherein the nucleic acid is selected from the group consisting of SEQ ID NOs. 185,397-185,409.

31. A method for identifying small RNA molecules that are conserved across more than one species, the method comprising:
a) creation of a genome-wide small RNA library for a first species;
b) creation of a genomic library or genomic-wide small RNA library for a second species;
c) comparing the library of the first species to the library of the second species; and
d) identifying small RNA molecules found in both the first library and the second library.

32. The method of claim 31, wherein the first species is Arabidopsis thaliana.

33. The method of claim 32, wherein the second species is a member selected from the group consisting of a eukaryote, a plant, a fungi, a yeast, and a mammal.

34. A method for identifying small RNA molecules that are unique to a single species, the method comprising:
a) creation of a genome-wide small RNA library for a first species;
b) creation of a genomic library or genomic-wide small RNA library for a second species;
c) comparing the library of the first species to the library of the second species; and
d) identifying small RNA molecules found in only one of said libraries.

35. The method of claim 34, wherein the first species is Arabidopsis thaliana.

36. The method of claim 35, wherein the second species is a member selected from the group consisting of a eukaryote, a plant, a fungi, a yeast, and a mammal.
